(19) United States
(12) Patent Application Publication
(10) Pub. No.: US 2024/0142451 A1
(43) Pub. Date: May 2, 2024

(54) METHODS FOR CHARACTERIZING THE IMMUNE RESPONSE OF A SUBJECT TO A DENGUE VIRUS COMPOSITION

(71) Applicant: TAKEDA VACCINES, INC., Cambridge, MA (US)

(72) Inventors: Isamu TSUJI, Cambridge, MA (US); Hansi DEAN, Cambridge, MA (US); Mayuri SHARMA, Cambridge, MA (US); Christina DEMASO, Cambridge, MA (US); Michael EGAN, Cambridge, MA (US); Eduardo NASCIMENTO, Cambridge, MA (US)

(21) Appl. No.: 18/284,987

(22) PCT Filed: Mar. 31, 2022
(86) PCT No.: PCT/US2022/022822

§ 371 Date: Sep. 29, 2023

Related U.S. Application Data
(60) Provisional application No. 63/168,460, filed on Mar. 31, 2021.

(51) Int. Cl. G01N 33/569; G01N 33/543

(52) U.S. Cl. CPC G01N 33/56983; G01N 33/54373; G01N 2333/185; G01N 2469/20; G01N 2470/04

(57) ABSTRACT

The present invention relates to a method for characterizing the immune response of a subject to a tetravalent dengue virus composition by performing the method for determining affinity, binding kinetics and/or concentration of an antibody or of an antibody mixture and at least one other method. In a further embodiment, the present invention relates to a method for characterizing the immune response of a subject to a virus-containing vaccine composition by performing a combination of assays. In a further embodiment, the present invention relates to a method for predicting protective efficacy of a dengue vaccine candidate. In another embodiment the present invention relates to a method for preparing a vaccine formulation.

### TECHNICAL FIELD

The present invention relates to methods for determining affinity, binding kinetics and/or concentration of an antibody or of an antibody mixture specific for a virus using virus-like particles (VLPs) and/or live viruses or inactivated viruses attached to biosensors. Further, the present invention relates to the VLPs and live or inactivated viruses attached to biosensors and methods for producing them. In another embodiment, the present invention relates to a method for characterizing the immune response of a subject to a tetravalent dengue virus composition by performing the method for determining affinity, binding kinetics and/or concentration of an antibody or of an antibody mixture disclosed herein and at least one other method. In a further embodiment, the present invention relates to a method for characterizing the immune response of a subject to a virus-containing vaccine composition by performing a combination of assays. In a further embodiment, the present invention relates to a method for predicting the protective efficacy of a dengue vaccine candidate.

### BACKGROUND OF THE INVENTION

Antibody affinity maturation is the process whereby the immune system generates antibodies of higher affinities during a response to antigen through somatic hypermutation. Antibody somatic hypermutation takes place in germinal centers after exposure to antigen, either by infection or immunization. B cells in the germinal centers express enzymes which insert point mutations throughout the Ig heavy and light chains. The repertoire of mutated B cells is then selected and enriched for high affinity of the antibody to its cognate antigen. Iterative rounds of selection and proliferation of somatically mutated clonal variants result in a population of antibodies that are enriched for higher affinity binders, based on successive accumulation of somatic mutations over time. Collectively, antibodies with increased affinities after antigen exposure contribute to an overall increase in polyclonal antibody avidity.

The importance of antibody affinity maturation to effective antiviral responses is well established. For example, HIV antibody affinity correlates with neutralization potency and breadth. Affinity maturation of B cells specific for conserved epitopes after sequential exposure to infection is required for protection from re-infection by diverse influenza viruses and is required to generate mAbs of sufficient potency for Ebola virus therapy. A common theme of successful antiviral immunity is induction of high affinity functional antibodies to conserved epitopes, in the context of abundant ineffective immune responses to variable viral epitopes. Information on antibody affinity maturation during dengue infections is limited, but available studies point to the potential role of affinity matured antibodies in resistance to post-secondary dengue infections. After a second dengue infection, neutralizing antibodies were boosted to the previous infecting serotype and cross-neutralizing antibodies to all serotypes increase. Over time, neutralizing antibody response magnitude decreases, but breadth of neutralizing antibodies is maintained, therefore the quantity of antibody decreases, but the quality of neutralizing antibodies is stably altered. After an individual has had 2 dengue infections, a third infection is rarely symptomatic, suggesting that immune responses elicited by a second dengue infection are protective. Repeated DENV infections have been shown to increase monoclonal and polyclonal antibody avidity and increased neutralization potency.

Affinity maturation leads to antibodies of higher affinity and avidity, required for optimal antiviral functions, including virus neutralization and antibody-dependent cell-mediated cytotoxicity. Sometimes, an increase of antiviral immunity in an individual is mainly due to the activity of a single affinity matured antibody, but it is generally believed to be mediated by the combined effect of multiple affinity matured antibodies that are present in the circulation.

The titer of neutralizing antibodies in serum is the most common measure of antibody responses to vaccination and infection. However, neutralizing antibodies do not always correlate with vaccine efficacy and neutralization assays do not measure all antibody effector functions. The degree of affinity maturation driven by vaccination or natural infection is an important parameter to be measured.

It is of particular importance to measure antibodies directed to particulate antigens, since said particulate antigens preserve the conformational and quaternary epitopes being the targets of many potent neutralizing antibodies. For example, it is known that for Dengue virus, neutralizing antibodies generally bind only to the complete virus or virus-like particles (VLPs), but not to the isolated envelope proteins or peptides. Metz et al. reported weak reactivity of quaternary epitope antibodies to recombinant envelope proteins, because the envelope protein formed monomers, PLOS Neglected tropical diseases 12 (2018) e0006793.

In a study by Lau et al., J. Clin. Virol. 69 (2015), 63-67 the antibody avidity of antibodies to dengue virus type 2 virions was determined. The antibodies were detected by an enzyme-linked immunosorbent assay (ELISA) conducted in the presence of chaotropic reagents such as 8M urea in order to reduce the non-specific binding. The described ELISA assay could not be reproduced by other laboratories. While the described ELISA method provides high throughput, the method lacks accuracy. The method is prone to select strong binding antibodies over antibodies with weaker binding affinity.

Thus, there remains a need for an assay suitable for the determination of the binding affinity or avidity of antibodies specific for viruses. In particular, the assay shall be suitable for the determination of the binding affinity or avidity of antibodies or antibody mixtures directed to particulate antigens preserving conformational and quaternary epitopes. In combination with other assays the assay of the invention may be used to characterize the immune response of a subject to the administration of a dengue virus composition and to establish a correlate of protection. A further technical problem is the provision of a combination of assays for characterizing the immune response of a vaccine.

### SUMMARY OF THE INVENTION

The technical problems underlying the invention are solved by the provision of the subject-matter as defined in the claims.

According to a first aspect is provided a method for determining affinity, binding kinetics and/or concentration of an antibody or of an antibody mixture specific for a virus comprising the following steps:
a) providing a virus-like particle (VLP) attached to a biosensor, wherein said VLP comprises structural proteins from said virus;
b) contacting the VLP attached to the biosensor with a first solution containing the antibody or antibody mixture specific for the virus such that the antibody or antibody mixture binds to the VLP attached to the biosensor and measuring the association of the binding complex;
c) contacting the VLP attached to the biosensor having bound the antibody or antibody mixture with a second solution lacking the antibody or antibody mixture such that the antibody or antibody mixture dissociates from the VLP attached to the biosensor and measuring the dissociation of the binding complex, wherein the measuring in steps b) and c) are performed by surface plasmon resonance or biolayer interferometry; and
d) calculating the affinity, binding kinetics and/or concentration of the antibody or the antibody mixture specific for the virus from the measurement data in steps b) and c).

According to a second aspect is provided a method for determining affinity, binding kinetics and/or concentration of an antibody or of an antibody mixture specific for a virus comprising the following steps:
a) providing a live virus or an inactivated virus attached to a biosensor;
b) contacting the live virus or inactivated virus attached to the biosensor with a first solution containing the antibody or antibody mixture specific for the virus such that the antibody or antibody mixture binds to the live virus or inactivated virus attached to the biosensor and measuring the association of the binding complex;
c) contacting the live virus or inactivated virus attached to the biosensor having bound the antibody or antibody mixture with a second solution lacking the antibody or antibody mixture such that the antibody or antibody mixture dissociates from the live virus or inactivated virus attached to the biosensor and measuring the dissociation of the binding complex, wherein the measuring in steps b) and c) are performed by surface plasmon resonance or biolayer interferometry; and
d) calculating the affinity, binding kinetics and/or concentration of the antibody or the antibody mixture specific for the virus from the measurement data in steps b) and c).

According to a third aspect is provided a method for determining the avidity and/or affinity over time of an antibody or antibody mixture produced after immunization of a human subject with a virus vaccine comprising the following steps:
a) obtaining serum samples from said subject at different time points after immunization;
b) purifying the antibody or antibody mixture from the serum samples by affinity chromatography using Protein A, Protein G, Protein A/G, Protein L or anti-human IgG;
c) determining the avidity and/or affinity of the antibody or the antibody mixture specific for the virus in accordance with the method according to the present invention; and
d) assessing the avidity and/or affinity of the antibody or antibody mixture as a function of time.

According to a fourth aspect is provided a method for preparing a virus-like particle (VLP) attached to a biosensor, wherein said VLP comprises structural proteins from said virus, wherein the method comprises attaching the VLP to the biosensor by any of the following:
i) a pair of binding molecules capable of specifically binding to each other, wherein the first binding molecule is linked to the VLP and the second binding molecule is attached to the surface of the biosensor; and/or
ii) a covalent linkage of the VLP to a capture reagent attached to the biosensor.

According to a fifth aspect is provided a VLP attached to the biosensor which is obtainable by the method of the present invention.

According to a sixth aspect is provided a method of preparing a live virus or an inactivated virus attached to a biosensor, wherein the method comprises attaching said live virus or said inactivated virus to the biosensor by hydrophobic interaction of said live virus or said inactivated virus with a capture reagent linked to the surface of the biosensor.

According to a seventh aspect is provided the live virus or inactivated virus attached to the biosensor.

According to an eighth aspect is provided a method for characterizing the immune response of a subject to a tetravalent dengue virus composition administered to said subject, comprising performing with a serum sample from said subject the method of the first, second or third aspect and at least one other method selected from the group consisting of:
(a) a method to determine the level of neutralizing antibodies in said sample;
(b) a method comprising depleting antibodies against one dengue serotype from said sample followed by determining in the depleted sample the level of neutralizing antibodies against at least one serotype different from the dengue serotype used for depleting the antibodies, to detect the presence of type-specific and cross-reactive neutralizing antibodies in said sample;
(c) a method to quantitate the level of antibodies against a non-structural protein 1 of dengue virus in said sample;
(d) a method to quantitate the level of dengue-binding antibodies in said sample and
(e) a method to determine the presence and/or amount of flavivirus-reactive complement-fixing antibodies in said sample.

According to a ninth aspect is provided a method for characterizing the immune response of a subject to a virus-containing vaccine composition administered to said subject, comprising performing with a serum sample from said subject at least two methods selected from the group consisting of:
(a) a method to determine the level of neutralizing antibodies in said sample;
(b) a method comprising depleting antibodies against one dengue serotype from said sample followed by determining in the depleted sample the level of neutralizing antibodies against at least one serotype different from the dengue serotype used for depleting the antibodies, to detect the presence of type-specific and cross-reactive neutralizing antibodies in said sample;
(c) a method to determine the level of antibodies against a non-structural protein 1 of dengue virus in said sample;
(d) a method to determine the level of dengue-binding antibodies in said sample;
(e) a method to determine the presence and/or amount of flavivirus-reactive complement-fixing antibodies in said sample; and
(f) the avidity assay method according to the present invention.

In a tenth aspect the present invention provides a method for predicting the protective efficacy of a dengue vaccine candidate comprising
determining the presence and/or amount of at least two immune response parameters selected from the group consisting of neutralizing antibodies, serotype specific and/or cross-reactive neutralizing antibodies, complement-fixing antibodies, dengue total binding antibodies, high affinity binding antibodies against dengue virus and antibodies against dengue non-structural protein 1 in a blood sample from a subject vaccinated with the dengue vaccine candidate, and
predicting the dengue vaccine candidate to provide protective efficacy if the presence of at least two immune response parameters selected from the group consisting of neutralizing antibodies, serotype specific and/or cross-reactive neutralizing antibodies, complement-fixing antibodies, dengue total binding antibodies, high affinity binding antibodies against dengue virus and antibodies against dengue non-structural protein 1 (NS1) is determined in the blood sample.

In an eleventh aspect the present invention provides a method for preparing a vaccine formulation comprising
performing the method for predicting the protective efficacy of a dengue vaccine candidate according to the present invention; and
formulating the vaccine candidate predicted to provide protective efficacy with a pharmaceutically acceptable excipient.

In a twelfth aspect the present invention provides a vaccine formulation obtainable by the method for preparing the vaccine formulation in accordance with the present invention.

Surprisingly, the inventors have found that methods for detecting and monitoring biological interactions in real-time such as surface plasmon resonance (SPR) technology or biolayer interferometry (BLI) can be successfully applied in the determination of binding parameters such as the avidity index of antibodies directed to particulate antigens including quaternary and conformational epitopes. According to the invention the particulate antigen, in particular the live virus or the VLP, is attached to the biosensor. This allows the analysis of complex antibody mixtures from patient samples or vaccinated individuals.

Kumar et al., Biosensors 6 (2016), pages 1 to 16 discloses the application of SPR technology for the analysis of binding of live viruses to a biosensor being modified with a glycan, a virus-specific antibody or an aptamer. Such a system, however, does not allow the assessment of the binding parameters of antibodies from samples or vaccinated individuals. The prior art therefore rather teaches away from the direct coupling of the live virus or the VLP to the biosensor surface.

The present inventors have further found that the avidity index, i.e. the ratio of response/dissociation rate (k_{off}) for antibodies or antibody mixtures from vaccinated individuals, calculated from the SPR or BLI measurement data is useful for assessing the efficacy of the vaccine in vivo. Thus, the SPR or BLI measurement can be used for an in vitro assessment of the affinity maturation of the antibodies in the vaccinated individuals over time.

The present inventors have further found that a combination of assays measuring diverse aspects of the vaccine-driven immune response, in particular antibody-based immune response parameters in addition to neutralizing antibody responses, are important in characterizing the protective efficacy of a dengue vaccine candidate. As a result of exploratory immunology of subjects vaccinated with tetravalent Dengue vaccine (TDV; Takeda) the present inventors claim that the combination of particular immune response parameters may have predictive value for the protective efficacy of dengue vaccine candidates.

### BRIEF DESCRIPTION OF THE DRAWINGS

FIG. 1 shows the plate layout.

FIG. 2 shows the biosensor plate layout. SA means streptavidin biosensor.

FIG. 3 shows an SDS-PAGE analysis of anti-DENV Ab purified from DEN203 sera sample.

FIG. 4 shows the optimization of Dengue VLP biotinylation.

FIG. 5 shows the Biosensor image of dengue vaccine immunized patient, ID10122005.

FIG. 6 shows the Biosensor image of dengue vaccine immunized patient, ID1044010.

FIG. 7 shows the changes in avidity of DENV1 specific antibodies for immunized patients.

FIG. 8 shows the changes in avidity of DENV2 specific antibodies for immunized patients.

FIG. 9 shows the changes in avidity of DENV3 specific antibodies for immunized patients.

FIG. 10 shows the changes in avidity of DENV4 specific antibodies for immunized patients.

FIG. 11 shows the results of avidity assay of Dengue live virus serotype 3. FIG. 11A relates to IgG from negative control sera 250 ug/mL. FIG. 11B relates to IgG from 1081012 250 ug/mL. FIG. 11C relates to IgG from positive control sera 250 ug/mL. FIG. 11D relates to IgG from 1082004 250 ug/mL. FIG. 11E relates to IgG from 1073001 D90 250 ug/mL.

FIG. 12 shows the result of an anti-NS1 IgG ELISA with serum samples from subjects treated with a tetravalent composition comprising live attenuated dengue viruses of serotypes 1, 2, 3 and 4. ****: p<0.0001, paired t-test

FIG. 13 shows the result of an anti-NS1 IgG ELISA with serum samples from DENV seropositive and DENV seronegative subjects at baseline treated with a tetravalent composition comprising live attenuated dengue viruses of serotypes 1, 2, 3 and 4. ****p<0.0001, ***p<0.0005, **p<0.005, *p<0.05, paired t-test

FIG. 14 shows the result of an dengue total binding IgG ELISA on serum samples from participants collected before (day 0) and after treatment with a tetravalent composition comprising live attenuated dengue viruses of serotypes 1, 2, 3 and 4 (days 120 and 180) performed as a paired t-test. ****; p<0.0001. White symbols: Baseline seronegative by MNT; Black filled symbols: Baseline seropositive by MNT.

FIG. 15 shows the correlation between avidity index and MNT antibody titer for k_{off} divided subjects. a Correlation analysis using all data sets. DENV-1: black circles relate to Log 10[k_{off} −4.6 −4.2], unfilled triangles relate to Log 10[k_{off} −4.2 −3.9] and crosses relate to Log 10[k_{off} −3.9 −1.9], DENV-2: black circles relate to Log 10[k_{off} −4.7 −4.6], unfilled triangles relate to Log 10[k_{off}—−4.6−4.0] and crosses relate to Log 10[k_{off}—−4.0 −2.8], DENV-3: black circles relate to Log 10[k_{off} −4.7 −4.6], unfilled triangles relate to Log 10[k_{off}—−4.6 −4.0] and crosses relate to Log 10[k_{off}—−4.0 −1.9], DENV-4: black circles relate to Log 10[k_{off} −4.7 −4.6], unfilled triangles relate to Log 10[k_{off}—−4.6 −3.8] and crosses relate to Log 10[k_{off} −3.8 −2.6], b Correlation analysis data divided into three ranges by Log 10 k_{off} values. DENV-1: Log 10[k_{off} −4.6 −4.2: −4.2 −3.9: −3.9 −1.9], DENV-2: Log 10[k_{off} −4.7 −4.6: −4.6 −4.0: −4.0 −2.8], DENV-3: Log 10[k_{off} −4.7 −4.6: −4.6 −4.0: −4.0 −1.9], DENV-4: Log 10[k_{off} −4.7 −4.6: −4.6 −3.8: −3.8 −2.6]. All baseline seronegative and seropositive volunteer data were used and data under response LoD were eliminated from the analysis: DENV-1: 0.017, DENV-2: 0.015, DENV-3: 0.018, DENV-4: 0.014. LoD: Limit of Detection, Correlation analysis date are shown in Table 10.

FIG. 16, first panel shows the determination of the concentration of dengue total binding IgG antibodies in DEN-304 clinical samples. Serum samples from 48 baseline seronegative by MNT at Day 1 pre-vaccination, and Day 120 and Day 270 post-vaccination, were assessed for dengue total binding IgG response elicited by TDV against DENV-1, DENV-2, DENV-3 and DENV-4. Concentrations of dengue total binding IgG are shown as Tukey's box plots representing median and inter-quartile distance. Seronegative subjects are shown in white boxes and baseline seropositive subjects are depicted as grey hatched boxes. MNT: microneutralization test, RU: Relative Units. FIG. 16, second panel shows the determination of the concentration of dengue total binding IgG antibodies in DEN-301 clinical samples. Serum samples from 24 baseline seronegative by MNT at Day 1 pre-vaccination, Day 120, Day 270, and Day 450 post-vaccination, were assessed for dengue total binding IgG response elicited by TDV against DENV-1, DENV-2, DENV-3 and DENV-4. Concentrations of dengue total binding IgG are shown as Tukey's box plots representing median and inter-quartile distance. Seronegative subjects are shown in white boxes and baseline seropositive subjects are depicted as grey hatched boxes. MNT: microneutralization test, RU: Relative Units.

FIG. 17, first panel shows the summary of anti-Dengue IgG avidity assay of DEN-304 BL seronegative volunteers. Avidity index, Box and whisker plot: bar: min and max, Box: 25 and 75% percent tile, line: median of data from 48 BL seronegative volunteers. Serum samples collected on study Day 1 (baseline/pre-vaccination; first TAK-003 dose administered), Day 120 (1 month after administration of second dose) and Day 270: Avidity index=response/k_{off} Negative and zero values of Response and Avidity index were extrapolated to 0.001 and 1, respectively for drawing purpose. FIG. 17, second panel shows the summary of anti-dengue IgG avidity assay of DEN-301 BL seronegative volunteers (second panel). Avidity index, Box and whisker plot: bar: min and max, Box: 25 and 75% percent tile, line: median of data from 24 BL seronegative volunteers. Serum samples collected on study Day 1 (baseline/pre-vaccination; first TAK-003 dose administered), 120 (1 month after administration of second dose), 270 and 450: Avidity index=response/k_{off}. Negative and zero values of Response and Avidity index were extrapolated to 0.001 and 1, respectively for drawing purpose.

FIG. 18, first panel shows the determination of complement-fixing antibodies in baseline seronegative subjects in the DEN-304 clinical study. Sequential serum samples from Days 1, 120 and 270 from 48 randomly selected baseline seronegative subjects in DEN-304 were tested for kinetics of production of anti-DENV complement-fixing antibodies following the first and second dose of TDV on Day 0 and Day 90, respectively. Anti-DENV complement-fixing antibody titers in arbitrary units/mL [EU/mL] are shown as geometric mean with 95% CI. FIG. 18, second panel shows the determination of complement-fixing antibodies in baseline seronegative subjects in the DEN-301 clinical study. Sequential serum samples from Days 1, 120, 270 and 450 from 24 randomly selected baseline seronegative subjects in DEN-301 (Asia Pacific) were tested for kinetics of production of anti-DENV complement-fixing antibodies following the first and second dose of TDV on Day 0 and Day 90, respectively. Anti-DENV complement-fixing antibody titers in arbitrary units/mL [EU/mL] are shown as geometric mean with 95% CI.

FIG. 19, first panel shows the determination of anti-DENV-2 NS1-specific and cross-reactive IgG antibodies in adult and children in the DEN-304 clinical study. Serum samples from 48 baseline seronegative subjects at Day 1 pre-vaccination and Day 120 and Day post-vaccination were assessed for anti-dengue NS1 IgG response elicited by TDV against DENV-1 NS1, DENV-2 NS1, DENV-3 NS1 and DENV-4 NS1. Concentrations of anti-dengue NS1 IgG are shown as Tukey's box plots representing median and inter-quartile distance. Abbreviations: NS1, nonstructural protein 1. FIG. 19, second panel shows the determination of anti-DENV-2 NS1-specific and cross-reactive IgG antibodies in adult and children in the DEN-301 clinical study. Serum samples from 24 baseline seronegative TDV recipients at Day 1 pre-vaccination and Days 120, 270 and 450 post-vaccination, were assessed for anti-dengue NS1 IgG response elicited by TDV against DENV-1, DENV-2, DENV-3 and DENV-4 NS1. Concentrations of anti-dengue NS1 IgG are shown as Tukey's box plots representing median and inter-quartile distance. Abbreviations: APAC, Asia Pacific; NS1, nonstructural protein 1; RU, relative units.

FIG. 20 shows the concentration of anti-dengue NS1 antibodies induced by vaccination with the vaccine TAK-003 over time. The blood samples were collected from the vaccinated individuals at different time points during the clinical trial DEN-203.

FIG. 21 shows a correlation analysis obtained by performing linear regression of the log 10-transformed concentration between anti-dengue complement-fixing antibody levels and microneutralization (MNT value), total IgG binding and magnitude of affinity, respectively, after vaccination of individuals with TAK-003. The correlation analysis was performed with the statistical software JMP version 15.2 (SAS Institute).

### DETAILED DESCRIPTION OF THE INVENTION

Where the term “comprise” or “comprising” is used in the present description and claims, it does not exclude other elements or steps. For the purpose of the present invention, the term “consisting of” is considered to be an optional embodiment of the term “comprising”. If hereinafter a group is defined to comprise at least a certain number of embodiments, this is also to be understood to disclose a group which optionally consists only of these embodiments.

Where an indefinite or a definite article is used when referring to a singular noun e.g. “a” or “an”, “the”, this includes a plural form of that noun unless specifically stated. Vice versa, when the plural form of a noun is used, it refers also to the singular form.

Furthermore, the terms first, second, third or (a), (b), (c) and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

In the context of the present invention any numerical value indicated is typically associated with an interval of accuracy that the person skilled in the art will understand to still ensure the technical effect of the feature in question. As used herein, the deviation from the indicated numerical value is in the range of ±10%, and preferably of ±5%. The aforementioned deviation from the indicated numerical interval of ±10%, and preferably of ±5% is also indicated by the terms “about” and “approximately” used herein with respect to a numerical value.

According to a first aspect is provided a method for determining affinity, binding kinetics and/or concentration of an antibody or of an antibody mixture specific for a virus comprising the following steps:
a) providing a virus-like particle (VLP) attached to a biosensor, wherein said VLP comprises structural proteins from said virus;
b) contacting the VLP attached to the biosensor with a first solution containing the antibody or antibody mixture specific for the virus such that the antibody or antibody mixture binds to the VLP attached to the biosensor and measuring the association of the binding complex;
c) contacting the VLP attached to the biosensor having bound the antibody or antibody mixture with a second solution lacking the antibody or antibody mixture such that the antibody or antibody mixture dissociates from the VLP attached to the biosensor and measuring the dissociation of the binding complex, wherein the measuring in steps b) and c) are performed by surface plasmon resonance or biolayer interferometry; and
d) calculating the affinity, binding kinetics and/or concentration of the antibody or the antibody mixture specific for the virus from the measurement data in steps b) and c).

According to a second aspect is provided a method for determining affinity, binding kinetics and/or concentration of an antibody or of an antibody mixture specific for a virus comprising the following steps:
a) providing a live virus or an inactivated virus attached to a biosensor;
b) contacting the live virus or inactivated virus attached to the biosensor with a first solution containing the antibody or antibody mixture specific for the virus such that the antibody or antibody mixture binds to the live virus or inactivated virus attached to the biosensor and measuring the association of the binding complex;
c) contacting the live virus or inactivated virus attached to the biosensor having bound the antibody or antibody mixture with a second solution lacking the antibody or antibody mixture such that the antibody or antibody mixture dissociates from the live virus or inactivated virus attached to the biosensor and measuring the dissociation of the binding complex, wherein the measuring in steps b) and c) are performed by surface plasmon resonance or biolayer interferometry; and
d) calculating the affinity, binding kinetics and/or concentration of the antibody or the antibody mixture specific for the virus from the measurement data in steps b) and c).

“Virus” herein means any virus including double-stranded and single-stranded DNA viruses, and double and single-stranded RNA viruses. Preferably, the virus may be a flavivirus or a calicivirus. Among the flaviviruses, preferred are Dengue virus, Japanese encephalitis virus, Tick-borne encephalitis virus, West Nile virus, Yellow fever and Zika virus. Particularly preferred, the flavivirus may be a Dengue virus or a Zika virus. Most preferred, the virus may be a dengue virus subtype selected from DENV-1, DENV-2, DENV-3 and DENV-4. Among the caliciviruses, Norovirus is preferred.

The virus may be a live virus capable of replication. The virus may be a wild-type or a live attenuated virus. “Wild-type virus” refers to the phenotype of the typical form of a virus as it occurs in nature. “Live attenuated virus” refers to a weakened, less vigorous virus as compared to the wild-type form of the virus which is still viable and able to replicate. An attenuated virus may be used to produce a vaccine that is capable of stimulating an immune response.

Attenuation may be achieved by serial passaging of the virus in a foreign host such as in tissue culture, embryonated eggs or live animals. Alternatively, attenuation may be performed by chemical agents.

The viruses include recombinant variants such as chimeric viruses. The term “recombinant virus” is generally used for a genetically modified virus that carries nucleotide sequences from a viral or non-viral species which are not present in the wild-type virus. A “chimeric virus” is generally used for a recombinant virus that consists of a combination of the genomes of two parent viruses and which may display biological properties characteristic for both parent viruses.

The virus may also be an inactivated virus. Virus inactivation renders the viruses inactive, or unable to infect. Suitable methods for virus inactivation include solvent/detergent inactivation, treatment with chemical agents such as formalin and beta-propiolactone, heating and/or acidic pH inactivation. Inactivation methods are known to the person skilled in the art.

“Antibody or antibody mixture specific for a virus” herein includes antibodies of any source or synthetically prepared antibodies. The antibody may be a human or animal antibody. Preferably, the antibody is a human antibody. The antibody may be of any subtype including IgG and IgM, with IgG being preferred. The antibody may be generated in vitro or in vivo. The antibodies may be generated by immunization of individuals using vaccines comprising a live attenuated virus, an inactivated virus and/or a virus like particle (VLP) or viral proteins or peptides thereof. The vaccine may further include adjuvants known in the art.

The antibodies or antibody mixture may also be obtained from samples from virus infected patients. Samples from whole blood or serum are preferred, most preferred the samples are from serum. The obtained sample may be purified before the use in the method according to the invention. Suitable antibody purification methods such as ion exchange chromatography, affinity chromatography or hydrophobic chromatography are known to the person skilled in the art.

“Affinity” describes the strength of the interaction between two biomolecules such as an antigen and an antibody specific for the antigen. Extremely strong interactions can be in the picomolar range, while weak interactions can be in the millimolar range. The dissociation constant (K_{D}) is the concentration of analyte at which half of all binding sites are occupied (at equilibrium conditions).

“Binding kinetics” relates to the rate at which the binding sites at a molecule such as an antibody are occupied with the ligand molecules such as antigens, i.e. the formation of the binding complex (association rate kₒₙ) and to the rate at which the ligand molecules are released from the binding sites, i.e. the dissociation of the binding complex (dissociation rate k_{off}). According to a preferred embodiment the association rate kₒₙ is measured when the binding sites attached to the biosensor are contacted with a solution containing the ligand molecules. According to a preferred embodiment the dissociation rate k_{off} is measured when the biosensor with the binding complex is removed from the above solution and introduced into a solution which does not contain the ligand molecules such as a buffer solution.

“Concentration” is the abundance of a constituent such as an antibody in a mixture divided by the total volume of the mixture. Preferably, the concentration is the molar concentration defined as the amount of a constituent (in moles) divided by the volume of the mixture.

A “virus-like particle (VLP)” closely resembles a virus, but is non-infectious, since it does not contain genetic material. The VLP can be naturally occurring or synthesized through the individual expression of viral structural proteins, which can then self-assemble into the virus-like structure. Combinations of structural capsid proteins can be used to create recombinant VLPs. VLPs have been produced from components of a wide variety of virus families VLPs can be produced in multiple cell culture systems including bacteria, mammalian cell lines, insect cell lines, yeast and plant cells. For a review on VLPs reference is made to Zeltins, Mol. Biotechnology 53 (2013), 92-107. VLPs can also be commercially obtained from companies such as the company Native Antigen.

“Biosensors” are devices used to detect the presence or concentration of a biological analyte, such as a biomolecule, a biological structure or a microorganism. Biosensors consist of three parts: a component that recognizes the analyte and produces a signal, a signal transducer, and a reader device. As used herein biosensors are suitable for use in connection with surface plasmon resonance (SPR) or biolayer interferometry (BLI) devices.

SPR is the resonant oscillation of conduction electrons at the interface between negative and positive permittivity material stimulated by incident light. Unlike many other immunoassays, such as ELISA, an SPR immunoassay is label free in that a label is not required for detection of the analyte. Additionally, the measurements on SPR can be followed in real-time allowing the monitoring of individual steps in sequential binding events. Useful systems in accordance with the present invention include Biacore® and IBIS® SPR systems.

BLI is a label-free technology for measuring biomolecular interactions. It is an optical analytical technique that analyzes the interference pattern of white light reflected from two surfaces: a layer of immobilized protein on the biosensor tip, and an internal reference layer. The binding between a ligand immobilized on the biosensor tip surface and an analyte in solution produces an increase in optical thickness at the biosensor tip, which results in a wavelength shift, Δλ, which is a direct measure of the change in thickness of the biological layer. Interactions are measured in real time, providing the ability to monitor binding specificity, rates of association and dissociation, or concentration, with high precision and accuracy; see Abdiche et al., Anal. Biochemistry 377 (2008), 209-217. Useful BLI systems for use in the present invention are the Pall-Fortebio® Octet® systems and the Pall-Fortebio® Blitz® systems. The Octet® system is preferred.

The biosensors suitable in connection with SPR or BLI can be in array format.

Useful Biosensors with affinity surfaces for proteins or peptides are commercially available e.g. from the company ForteBio. Currently, biosensors with the following surface modifications are available aminopropylsilane, amine reactive 2G, super streptavidin, anti-human Fc-capture, anti-mouse Fc-capture, streptavidin, anti-human IgG Fc, anti-murine IgG-Fv, anti-Penta-His, anti-His, Protein A, Protein G, Protein L, anti-human Fab-Ch1 2^{nd} generation, anti-GST and Ni-NTA (company Fortebio).

“Providing a VLP attached to biosensor” or “providing a live virus or inactivated virus attached to a biosensor” herein means that the VLP or the live virus or inactivated virus is immobilized on the surface of the biosensor by hydrophobic interactions or by covalent linkage. The immobilization can be direct or indirect such as mediated by binding partners.

For attaching a VLP to the biosensor any of the following is preferred:
i) a pair of binding molecules capable of specifically binding to each other, wherein the first binding molecule is linked to the VLP and the second binding molecule is attached to the surface of the biosensor; and/or
ii) a covalent linkage of the VLP to a capture reagent attached to the biosensor.

The pair of binding molecules is preferably selected from biotin/streptavidin; ligand/receptor; antigen/antibody; antibody/Protein A or Protein G; sugar/lectin; His-tag/Ni and sense/antisense oligonucleotides, particularly preferred the pair of binding molecules is biotin/streptavidin. Preferably, one member of said binding pairs is linked to the VLP by an activated moiety. Techniques for coupling of binding molecules to peptides and proteins are well-known in the art. Peptide coupling reagents include phosphonium reagents, uranium reagents, carbodiimide reagents, imidazolium reagents, organophosphorous reagents, acid halogenating reagents, chloroformate, pyridinium and other coupling reagents (see review article Han and Kim, Tetrahedron 60 (2004), 2447-2467).

Most preferred the VLP is biotinylated and the biosensor has streptavidin attached to its surface. Alternatively, it is preferred that the VLP is covalently linked to a biosensor having an amine-reactive surface such as the amine-reactive 2G biosensor commercially available from the company ForteBio.

For attaching the live virus or inactivated virus to the biosensor it is preferred that the attachment is mediated by hydrophobic interaction of the live virus or inactivated virus with a capture reagent linked to the surface of the biosensor. The capture reagent for attaching the live virus or the inactivated virus to the biosensor is preferably aminopropylsilane.

“Contacting the VLP attached to the biosensor with a first solution containing the antibody or antibody mixture specific for the virus such that the antibody or antibody mixture binds to the VLP attached to the biosensor” herein means that the antibody solution is contacted with the first solution under pH and salt conditions which allow the binding of the antibody to the VLP attached to the biosensor. Generally, the antibody will be present in a buffer system known in the art. Suitable buffers may be phosphate-buffered saline (PBS) or Tris-buffered saline (TBS). As outlined above for the VLP attached to the biosensor, the live virus attached to the biosensor or the inactivated virus attached to the biosensor may bind to the antibody or the antibody mixture specific for the virus under suitable pH and salt conditions. Generally, a buffer may also be used herein.

“Measuring the association of the binding complex” and “measuring the dissociation of the binding complex” herein includes the use of surface plasmon resonance (SPR) technology or biolayer interferometry (BLI) technology to measure the association and/or dissociation of the binding complex. For the present invention, the measuring by BLI is preferred. As discussed above, in BLI the association of the binding complex produces an increase in optical thickness at the biosensor tip which results in a measurable wavelength shift. In BLI the dissociation of the binding complex produces a decrease in optical thickness at the biosensor tip which results in a measurable wavelength shift. Thus, by recording the wavelength shift the association of the binding complex and/or the dissociation of the binding complex can be measured. The measurement is performed in real time, i.e. the association and/or dissociation can be followed over time.

“Calculating the affinity, binding kinetics and/or concentration of the antibody or the antibody mixture specific for the virus from the measurement data” herein means that the data obtained from the measurements using SPR or BLI are processed. Affinity calculations comprise the determination of the dissociation constant (K_{d}) or of the equilibrium constant (K_{eq}) for the binding of a ligand such as an antibody to a receptor such as a virus or VLP. Affinity calculations further include calculations known to the person skilled in the art for determining the effect of inhibitor binding. The influence of single and multiple binding sites may be calculated using e.g. the Scatchard Plot and the Hill Plot.

Binding kinetics calculations include the determination of the association rate (kₒₙ) and the dissociation rat (k_{off}) as outlined below. Further, binding kinetics calculations comprise calculations of the binding process such as single-step and two-step bimolecular binding processes.

These calculations can be performed by using commercially available software. Suitable software includes Octet Data Analysis Software from the company Fortebio.

In a further aspect a method is provided for determining the avidity and/or affinity over time of an antibody or antibody mixture produced after immunization of a human subject with a virus vaccine comprising the following steps:
a) obtaining serum samples from said subject at different time points after immunization;
b) purifying the antibody or antibody mixture from the serum samples by affinity chromatography using Protein A, Protein G, Protein A/G, Protein L, or anti-human IgG;
c) determining the avidity and/or affinity of the antibodies for the virus as a function over time in accordance with the method described herein.

“Purifying antibodies or antibody mixtures from the serum samples by affinity chromatography using Protein A, Protein G, Protein A/G, Protein L or anti-human IgG” generally comprises the binding of the sample containing the antibodies or antibody mixture to the Protein A, Protein G, Protein A/G, Protein L or anti-human IgG matrix, the washing of the matrix with the bound antibody or antibody mixture and the elution of the bound antibody or antibody mixture from the matrix. Suitable conditions for binding of the antibody sample to the matrix include using a buffer at a pH from 7 to 8, wherein the buffer is preferably physiologically buffered. The washing can be done using phosphate-buffered saline. For the elution an acidic elution buffer, e.g. 0.1M glycine-HCL, pH 2.8 may be used. After elution from the matrix, the purified antibody sample is neutralized. Neutralization of the eluted samples can be done e.g. using a 1M Tris-HCL (pH 8.0) buffer.

Preferably, the method determines the avidity index of the antibody or antibody mixture from serum samples obtained after different points of time after immunization.

During the maturation of the humoral immune response, there is an antibody selection process that results in synthesis of antibodies with increased antigen-antibody association strength.

“Avidity” refers to the accumulated strength of multiple affinities of individual non-covalent binding interactions, such as between an antibody and its antigen. Calculations for avidity may include Scatchard plots. Another measure for avidity may be the avidity index.

“Avidity index” as used herein is calculated as follows: Avidity index=antibody response/k_{off}, wherein k_{off} is the dissociation rate of the complex. The unit of k_{off} is s⁻¹. Antibody response herein means the amount of specific antibody generated in reaction to immunization with a given antigen. The amount of antibody may be determined by BLI or SPR. Alternatively, the amount of antibody may be measured using ELISA assays.

Generally, antibodies produced at an early stage during primary response to an infection have lower antigen avidity than those produced at a later stage. The SPR/BLI assays on the one hand and chaotrope-based assays on the other hand measure different aspects of antibody avidity, with the former characterising the kinetics of antibody-antigen interactions in relation to time and the latter describing resistance of antibody-antigen binding to disruption by chaotropic reagents.

Surprisingly, it has been found by the present inventors that the in vitro determination of the avidity index of antibody-containing samples from vaccinated individuals at different time points after vaccination is an indicator of the avidity of the in vivo generated antibodies over time and therefore for the efficacy of the used virus vaccine. Suitable time points may include at least three different time points over a period of at least 180 days, preferably over at least one year after vaccination.

In a preferred embodiment the virus vaccine is a tetravalent dengue virus composition comprising four live, attenuated dengue virus strains. More preferably, the four live, attenuated dengue virus strains are:
(i) a chimeric dengue serotype 2/1 strain,
(ii) a dengue serotype 2 strain,
(iii) a chimeric dengue serotype 2/3 strain, and
(iv) a chimeric dengue serotype 2/4 strain.

In a further preferred embodiment, each one of the four live, attenuated dengue virus strains has attenuating mutations in the 5′-noncoding region (NCR) at nucleotide 57 from cytosine to thymine, in the NS1 gene at nucleotide 2579 from guanine to adenine resulting in an amino acid change at position 828 of the NS1 protein from glycine to asparagine, and in the NS3 gene at nucleotide 5270 from adenine to thymine resulting in an amino acid change at position 1725 of the NS3 protein from glutamine to valine.

The four live, attenuated Dengue virus strains may be TDV-1, TDV-2, TDV-3 and/or TDV-4. The nucleotide and amino acid sequence of TDV-1 is set forth in SEQ ID NO:1 and SEQ ID NO:2, respectively. The nucleotide and amino acid sequence of TDV-2 is set forth in SEQ ID NO:3 and SEQ ID NO:4, respectively. The nucleotide and amino acid sequence of TDV-3 is set forth in SEQ ID NO:5 and SEQ ID NO:6, respectively. The nucleotide and amino acid sequence of TDV-4 is set forth in SEQ ID NO:7 and SEQ ID NO:8, respectively.

In one embodiment, TDV-2 comprises in addition to the three attenuating mutations one or more mutations selected from:
a) a mutation in the prM gene at nucleotide 524 from adenine to thymine resulting in an amino acid change at position 143 from asparagine to valine, and/or
b) a silent mutation in the E gene at nucleotide 2055 from cytosine to thymine, and/or
c) a mutation in the NS2A gene at nucleotide 4018 from cytosine to thymine resulting in an amino acid change at position 1308 from leucine to phenylalanine, and/or
d) a silent mutation in the NS3 gene at nucleotide 5547 from thymine to cytosine, and/or
e) a mutation in the NS4A gene at nucleotide 6599 from guanine to cytosine resulting in an amino acid change at position 2168 from glycine to alanine, and/or
f) a silent mutation in the prM gene at nucleotide 900 from thymine to cytosine. The silent mutation in the NS5 gene at nucleotide 8571 from cytosine to thymine of DEN-2 PDK-53 is not present in the TDV-2 strain.

In another embodiment, TDV-2 comprises in addition to the three attenuating mutations one or more mutations selected from:
g) a mutation in the prM gene at nucleotide 592 from adenine to guanine resulting in an amino acid change at position 166 from lysine to glutamine, and/or
h) a mutation in the NS5 gene at nucleotide 8803 from adenine to guanine resulting in an amino acid change at position 2903 from isoleucine to valine.

In one embodiment, TDV-1 comprises in addition to the three attenuating mutations one or more mutations selected from:
a) a mutation in the NS2A gene at nucleotide 4018 from cytosine to thymine resulting in an amino acid change at position 1308 from leucine to phenylalanine, and/or
b) a silent mutation in the NS3 gene at nucleotide 5547 from thymine to cytosine, and/or
c) a mutation in the NS4A gene at nucleotide 6599 from guanine to cytosine resulting in an amino acid change at position 2168 from glycine to alanine, and/or
d) a silent mutation in the E gene at nucleotide 1575 from thymine to cytosine, and/or
e) a silent mutation in the junction site between the prM-E gene and the DEN-2 PDK-53 backbone at nucleotide 453 from adenine to guanine, and/or
f) a mutation in the junction site between the prM-E gene and the DEN-2 PDK-53 backbone at nucleotides 2381/2382 from thymine-guanine to cytosine-cytosine resulting in an amino acid change at position 762 from valine to alanine.

In another embodiment, TDV-1 comprises in addition to the three attenuating mutations one or more mutations selected from:
g) a mutation in the NS2A gene at nucleotide 3823 from adenine to cytosine resulting in an amino acid change at position 1243 from isoleucine to leucine, and/or
h) a mutation in the NS2B gene at nucleotide 4407 from adenine to thymine resulting in an amino acid change at position 1437 from glutamine to asparagine, and/or
i) a silent mutation in the NS4B gene at nucleotide 7311 from adenine to guanine.

In one embodiment, TDV-3 comprises in addition to the three attenuating mutations one or more mutations selected from:
a) a mutation in the NS2A gene at nucleotide 4012 from cytosine to thymine resulting in an amino acid change at position 1306 from leucine to phenylalanine, and/or
b) a silent mutation in the NS3 gene at nucleotide 5541 from thymine to cytosine, and/or
c) a mutation in the NS4A gene at nucleotide 6593 from guanine to cytosine resulting in an amino acid change at position 2166 from glycine to alanine, and/or
d) a silent mutation in the junction site between the prM-E gene and the DEN-2 PDK-53 backbone at nucleotide 453 from adenine to guanine, and/or
e) a mutation in the junction site between the prM-E gene and the DEN-2 PDK-53 backbone at nucleotides 2375/2376 from thymine-guanine to cytosine-cytosine resulting in an amino acid change at position 760 from valine to alanine, and/or
f) a silent mutation in the prM gene at nucleotide 552 from cytosine to thymine, and/or
g) a mutation in the E gene at nucleotide 1970 from adenine to thymine resulting in an amino acid change at position 625 from histidine to leucine.

In another embodiment, TDV-3 comprises in addition to the three attenuating mutations one or more mutations selected from:

h) a mutation in the E gene at nucleotide 1603 from adenine to thymine resulting in an amino acid change at position 503 from threonine to serine, and/or
i) a silent mutation in the NS5 gene at nucleotide 7620 from adenine to guanine.

In one embodiment, TDV-4 comprises in addition to the three attenuating mutations one or more mutations selected from:
a) a mutation in the NS2A gene at nucleotide 4018 from cytosine to thymine resulting in an amino acid change at position 1308 from leucine to phenylalanine, and/or
b) a silent mutation in the NS3 gene at nucleotide 5547 from thymine to cytosine, and/or
c) a mutation in the NS4A gene at nucleotide 6599 from guanine to cytosine resulting in an amino acid change at position 2168 from glycine to alanine, and/or
d) a silent mutation in the junction site between the prM-E gene and the DEN-2 PDK-53 backbone at nucleotide 453 from adenine to guanine, and/or
e) a mutation in the junction site between the prM-E gene and the DEN-2 PDK-53 backbone at nucleotides 2381/2382 from thymine-guanine to cytosine-cytosine resulting in an amino acid change at position 762 from valine to alanine, and/or
f) a mutation in the C gene at nucleotide 396 from adenine to cytosine resulting in an amino acid change at position 100 from arginine to serine, and/or
g) a silent mutation in the E gene at nucleotide 1401 from adenine to guanine, and/or
h) a mutation in the E gene at nucleotide 2027 from cytosine to thymine resulting in an amino acid change at position 644 from alanine to valine, and/or
i) a mutation in the E gene at nucleotide 2275 from adenine to cytosine resulting in an amino acid change at position 727 from methionine to leucine.

In another embodiment, TDV-4 comprises in addition to the three attenuating mutations one or more mutations selected from:
j) a silent mutation in the C gene at nucleotide 225 from adenine to thymine, and/or
k) a mutation in the NS2A gene at nucleotide 3674 from adenine to guanine resulting in an amino acid change at position 1193 from asparagine to glycine, and/or
l) a mutation in the NS2A gene at nucleotide 3773 from adenine to an adenine/guanine mix resulting in an amino acid change at position 1226 from lysine to a lysine/asparagine mix, and/or
m) a silent mutation in the NS3 gene at nucleotide 5391 from cytosine to thymine, and/or
aa) a mutation in the NS4A gene at nucleotide 6437 from cytosine to thymine resulting in an amino acid change at position 2114 from alanine to valine, and/or
bb) a silent mutation in the NS4B gene at nucleotide 7026 from thymine to a thymine/cytosine mix, and/or
cc) a silent mutation in the NS5 gene at nucleotide 9750 from adenine to cytosine.

In a further aspect a method of preparing a virus-like particle (VLP) attached to a biosensor suitable for SPR or BLI is provided, wherein said VLP comprises structural proteins from said virus, wherein the method comprises attaching the VLP to the biosensor by any of the following:
i) a pair of binding molecules capable of specifically binding to each other, wherein the first binding molecule is linked to the VLP and the second binding molecule is attached to the surface of the biosensor; and/or
ii) a covalent linkage of the VLP to a capture reagent attached to the biosensor.

The VLPs and the pairs of binding molecules are as defined above. Preferably, the VLP is biotinylated and the biosensor has streptavidin attached to its surface. In a further preferred embodiment, the VLP is covalently linked to a biosensor having an amine-reactive surface.

The VLPs attached to the biosensors obtainable by the above methods are also encompassed by the present invention.

In a further aspect a method of preparing a live virus or an inactivated virus attached to a biosensor suitable for SPR or BLI is provided, wherein the method comprises attaching said live virus or said inactivated virus to the biosensor by hydrophobic interaction of said live virus or said inactivated virus with a capture reagent linked to the surface of the biosensor.

In a preferred embodiment said live virus or said inactivated virus is attached to the biosensor by hydrophobic interaction of said live virus or said inactivated virus with a capture reagent linked to the surface of the biosensor. More preferably, the capture reagent comprises aminopropylsilane.

The live virus or inactivated virus attached to the biosensors obtainable by the above methods are also encompassed by the present invention.

In a further aspect a method for characterizing the immune response of a subject to a tetravalent dengue virus composition administered to said subject is provided, comprising performing with a blood serum sample from said subject the method of the first, second or third aspect and at least one other method selected from the group consisting of:
(a) a method to determine the level of neutralizing antibodies in said sample;
(b) a method comprising depleting antibodies against one dengue serotype from said sample followed by determining in the depleted sample the level of neutralizing antibodies against at least one serotype different from the dengue serotype used for depleting the antibodies, to detect the presence of type-specific and cross-reactive neutralizing antibodies in said sample;
(c) a method to quantitate the level of antibodies against a non-structural protein 1 of dengue virus in said sample;
(d) a method to quantitate the level of dengue-binding antibodies in said sample; and
(e) a method to determine the presence and/or amount of flavivirus-reactive complement-fixing antibodies in said sample.

The blood serum sample is obtained by collecting blood from a human subject and separating the serum from the other components of the blood. The blood serum sample is obtained from a human subject to which a dengue virus composition has been administered. The dengue virus vaccine with which the subject has been vaccinated may be a tetravalent dengue virus composition as described above. In one embodiment, the blood serum sample is heat inactivated before use. In one embodiment, the blood serum sample is stored at a temperature of less than or equal to −60° C. In the method of the present invention serial dilutions of the blood serum samples are prepared. The serial dilution of the blood serum samples is the stepwise dilution of the blood serum samples according to a given dilution factor. In one embodiment, the blood serum samples are stepwise diluted two-fold from an initial 1:10 dilution.

The serum sample may be obtained from a subject which was seropositive or seronegative before treatment with the dengue virus composition. As used herein, “seronegative” or “seronaïve” means that the subject does not have neutralizing antibodies against any one of dengue serotypes DENV-1, DENV-2, DENV-3 and DENV-4 in the serum. A seronegative or seronaïve subject or subject population is defined by a neutralizing antibody titer of less than 10 for each one of the four dengue serotypes as measured by a plaque reduction neutralization test. A subject or subject population having a neutralizing antibody titer of equal to or more than 10 for at least one dengue serotype as measured by a plaque reduction neutralization test is defined as being “seropositive” with respect to said dengue serotype.

The method of (a) to determine the level of neutralizing antibodies in said sample may be a neutralization assay. The neutralization assay may comprise the following steps:
(i) seeding cells from a dengue-susceptible cell line and culturing the cells for a culture period;
(ii) preparing serial dilutions of the serum sample;
(iii) separately mixing the serially diluted serum samples prepared in step (b) with dengue serotype 1, dengue serotype 2, dengue serotype 3 and dengue serotype 4 to obtain separate mixtures for each dengue serotype and incubating the separate mixtures;
(iv) adding the separate mixtures prepared in step (c) to the cells seeded and cultured in step (a) and incubating the cells with the separate mixtures;
(v) providing an overlay for the cells incubated in step (d) and incubating the cells for an incubation period;
(vi) determining the number of plaques in each well and comparing the number of plaques in each well to an non-neutralized control to determine the level of neutralizing antibodies against each of dengue serotypes 1, 2, 3 and 4.

In one embodiment, the dengue-susceptible cell line used in step (i) is selected from Vero cells, LLC-MK2 cells, CV-1 cells and BHK-21 cells. Preferably, the dengue-susceptible cell line used in step (a) is a Vero cell line. The dengue-susceptible cell line is seeded on suitable plates such as 6-well, 24-well or 96-well plates, i.e. a defined amount of the dengue-susceptible cell line is introduced into a well of a plate which contains a suitable growth medium for the dengue-susceptible cell line. Suitable growth media for dengue-susceptible cell lines are known to the skilled person and include DMEM with 10% fetal bovine serum. The dengue-susceptible cell line is seeded with a density of 1 to 4×10⁵ cells per ml, preferably of 1.5 to 3.5×10⁵ cells per ml and more preferably of 2 to 3×10⁵ cells per ml. In some embodiments, the dengue-susceptible cell line is cultured for a culture period of 12 to 48 hours. The culture period is calculated from the time the cells are seeded until the time the separate mixtures of the serially diluted blood serum samples with dengue serotype 1, dengue serotype 2, dengue serotype 3 and dengue serotype 4 are added to the cells.

The dengue serotype strains with which the serially diluted blood serum samples are separately mixed are those strains from which the immunogenic components with which the subject has been vaccinated are derived. In one embodiment, the dengue serotype strains comprise one or more of the following: DENV-1 strain 16007, DENV-2 strain 16681, DENV-3 strain 16562 and DENV-4 strain 1036. In one embodiment, the subject has been vaccinated with a tetravalent dengue virus composition comprising a chimeric dengue serotype 2/1 strain comprising the prM and E genes of DENV-1 strain 16007, a dengue serotype 2 strain comprising the prM and E genes of DENV-2 strain 16681, a chimeric dengue serotype 2/3 strain comprising the prM and E genes of DENV-3 strain 16562, and a chimeric dengue serotype 2/4 strain comprising the prM and E genes of DENV-4 strain 1036.

The separate mixtures of the serially diluted blood serum samples with dengue serotype 1, dengue serotype 2, dengue serotype 3 and dengue serotype 4 prepared in step (c) may be incubated for 1 to 2 hours at a temperature of 37° C.

In one embodiment, in step (c) the dengue serotype 1 is DENV-1 strain 16007, dengue serotype 2 is DENV-2 strain 16681, dengue serotype 3 is DENV-3 strain 16562 and dengue serotype 4 is DENV-4 strain 1036.

The separate mixtures of the serially diluted blood serum samples with dengue serotype 1, dengue serotype 2, dengue serotype 3 and dengue serotype 4 prepared in step (c) are added to the dengue-susceptible cell line to allow for virus absorption. The cells are incubated with the separate mixtures of the serially diluted blood serum samples with dengue serotype 1, dengue serotype 2, dengue serotype 3 and dengue serotype 4 for a period of 60 to 180 minutes, preferably for a period of 90 to 120 minutes. The cells are incubated with the separate mixtures of the serially diluted blood serum samples with dengue serotype 1, dengue serotype 2, dengue serotype 3 and dengue serotype 4 for a period of 60 to 180 minutes, preferably for a period of 90 to 120 minutes at a temperature of 37° C.

The overlay provided in step (e) to the incubated cells serves to limit the virus diffusion within the plate which permits plaque formation. The overlay can be added to the cells either after aspiration of the separate mixtures of serially diluted blood serum samples with dengue serotype 1, dengue serotype 2, dengue serotype 3 and dengue serotype 4 or without aspiration of these mixtures. Preferably, the overlay is added to the cells without aspiration of the separate mixtures of serially diluted blood serum samples with dengue serotype 1, dengue serotype 2, dengue serotype 3 and dengue serotype 4. In one embodiment, the overlay in step (e) is selected from the group consisting of methylcellulose, carboxymethylcellulose and agarose. Preferably, the overlay is methylcellulose. The incubation periods used in step (e) may be adjusted based on the dengue serotype investigated.

In one embodiment, the number of plaques in each well is determined using serotype-specific anti-dengue monoclonal antibodies. The skilled person knows how to prepare serotype-specific antibodies. Suitable approaches are described for example in Gentry et al. (1982) Am. J. Trop. Med. Hyg. 31, 548-555; Henchal et al. (1985) Am. J. Trop. Med. Hyg. 34, 162-169; and Henchal et al. (1982) Am. J. Trop. Med. Hyg. 31(4):830-6). For example, mice can be immunized with a specific dengue serotype and the B cells isolated from these mice can be fused with a fusion partner to prepare a hybridoma. Suitable serotype-specific antibodies are selected based on the binding of the antibodies to the serotype with which the mice were immunized and lack of binding to those serotypes with which the mice were not immunized. In one embodiment, the mice were immunized with a serotype selected from dengue 1 strain Hawaii, Envelope, dengue 2 strain New Guinea C, Envelope, isotype 1, dengue 3 strain H87, Envelope, isotype 2A, and dengue 4 strain H241, Envelope, isotype 1.

To determine the number of plaques, the overlay is removed from the cells and the cells are washed, e.g. with phosphate-buffered saline. After washing, the cells are fixed with methanol or acetone for 60 minutes at a temperature of less than or equal to −20° C. After washing the cells, the serotype specific anti-dengue monoclonal antibodies are added to the corresponding wells and incubated for 18±4 hours at 2-8° C., before the cells are washed and incubated with a labelled secondary antibody binding to the serotype specific anti-dengue monoclonal antibodies for 90 to 120 minutes at 37° C. After washing, the substrate for the enzyme attached to the labelled secondary antibody is added and incubated for an appropriate period. If the secondary antibody is labelled with peroxidase, the substrate may be azino-bis(3-ethylbenzthiazoline-6-sulfonic acid) (ABTS).

The number of plaques may be determined visually or using a plaque counter such as the ViruSpot Plaque counter. The percentage neutralization reduction may be determined compared to the virus control and the MNT50 value may be calculated.

Suitable neutralization assays are disclosed e.g. in Osorio et al. (2014) Lancet Infect Dis. 14: 830-838, Rodrigo et al. (2009) Am. J. Trop. Med. Hyg. 80(1): 61-65, Vorndam and Beltran (2002) Am. J. Trop. Med. Hyg. 66(2): 208-212 and Jirakanjanakit et al. (1997) Transct. Roy. Soc. Trop. Med. Hyg. 91: 614-617.

The method of (b) is a two part method which first comprises depleting antibodies against one dengue serotype from the serum sample and then detecting and quantifying the neutralizing antibodies against at least one serotype different from the dengue serotype used for depleting the antibodies. By this method it is possible to distinguish between type-specific and cross-reactive neutralizing antibodies in said sample.

“Type-specific neutralizing antibodies” are antibodies which are specific for one dengue serotype, i.e. antibodies which are specific for dengue serotype 1, but which do not react with dengue serotype 2, dengue serotype 3 and serotype 4, or antibodies which are specific for dengue serotype 2, but which do not react with dengue serotype 1, dengue serotype 3 and serotype 4, or antibodies which are specific for dengue serotype 3, but which do not react with dengue serotype 2, dengue serotype 1 and serotype 4, or antibodies which are specific for dengue serotype 4, but which do not react with dengue serotype 2, dengue serotype 3 and serotype 1. The type-specific neutralizing antibodies bind to an epitope which is specific for this dengue serotype and which is not present in other dengue serotypes.

“Cross-reactive neutralizing antibodies” are antibodies which bind to at least two, at least three or all four dengue serotypes. The cross-reactive neutralizing antibodies bind to an epitope which is common to at least two, at least three or all four dengue serotypes. Preferably, the cross-reactive neutralizing antibodies bind to an epitope which is common to all four dengue serotypes.

“Depleting antibodies from said sample” means that antibodies which bind to a specific target are removed from the serum sample such that the depleted sample obtained by depleting the antibodies contains a lower amount of antibodies binding to said specific target. The depleted sample contains 50%, 40% or 30% or less of antibodies binding to said specific target compared to the serum sample, preferably the depleted sample contains 20%, 15% or 10% or less of antibodies binding to said specific target compared to the serum sample and more preferably the depleted sample contains 8%, 5% or 3% or less of antibodies binding to said specific target compared to the serum sample.

The antibodies are depleted from the sample by incubating them with their specific target. In the method used in the present invention the antibodies are preferably depleted by incubating them with purified dengue virus of a specific serotype or a virus-like particle of a specific serotype. In one embodiment the antibodies are depleted by incubating them with purified dengue serotype 2 virus.

In one embodiment the purified dengue virus or virus-like particle of a specific serotype, preferably the purified dengue serotype 2 virus, is coupled to beads which can be removed from the sample. In one embodiment the beads are agarose beads, polystyrene beads or magnetic beads, preferably the beads are magnetic beads.

The purified dengue virus of a specific serotype, preferably the purified dengue serotype 2 virus, is coupled to the beads using a monoclonal antibody which binds to the purified dengue virus of a specific serotype, preferably the purified dengue serotype 2 virus. Suitable antibodies are known to the skilled person and include both serotype-specific and cross-reactive antibodies. Suitable cross-reactive antibodies include 4G2 (Henchal et al. (1985) Am. J. Trop. Med. Hyg. 34: 162-169), 1M7 (Smith et al. (2014) J. Virol. 88: 12233-12241), 2H2 (Falconar (1999) Arch. Virol. 144(12): 2313-2330), All, B7, C8, C10 (Rouvinski et al. (2015) Nature 520: 109-113). The antibody is first coupled to the beads and then the purified dengue virus or virus-like particle of a specific serotype, preferably the purified dengue serotype 2 virus is added. To avoid nonspecific binding the beads are blocked with 1% BSA in PBS. After incubation of the antibody-conjugated beads with the purified dengue virus or virus-like particle of a specific serotype, preferably the purified dengue serotype 2 virus the beads are washed and cross-linked with formaldehyde under appropriate conditions to stabilize the interaction between the antibody and the purified dengue virus or virus-like particle of a specific serotype.

The beads are incubated with diluted serum samples and then the beads having the serum antibodies bound to the purified dengue virus or virus-like particle of a specific serotype, preferably the purified dengue serotype 2 virus are removed. If magnetic beads are used in the method, the serum antibodies bound to the purified dengue virus or virus-like particle of a specific serotype, preferably the purified dengue serotype 2 virus are removed with a magnet. If agarose or polystyrene beads are used in the method, the serum antibodies bound to the purified dengue virus or virus-like particle of a specific serotype, preferably the purified dengue serotype 2 virus are removed by centrifugation. The supernatant of the beads is subjected to one or more additional rounds of depletion using the steps described above after which the depleted sample is obtained. A suitable method for serum depletion is described in Metz et al. (2018) Virol. J. 15:60. Further suitable methods for depletion are outlined in Swanstrom J A et al., J Infect Dis. 2019 Jun. 19; 220(2):219-227; de Alwis et al., PLoS Pathog. 2014 Oct. 2; 10(10):e1004386; Collins et al., Emerg Infect Dis. 2017 May; 23(5):773-781; Henein et al. J Infect Dis. 2017 Feb. 1; 215(3):351-358. The disclosures of which are incorporated herein by reference.

After the depleted sample has been obtained, the level of neutralizing antibodies in the depleted sample can be determined using any suitable assay, including ELISA, neutralization assay and a reporter virus particle (RVP) assay. Preferably, an RVP assay is used. Reporter virus particles are replication-incompetent serotype-specific dengue viral particles which have the same structural proteins as the dengue virus serotype and therefore retain its antigenic determinants and which express a gene encoding a reporter protein upon infection of permissive cells. Suitable reporter proteins are known to the skilled person and include green fluorescent protein (GFP), luciferase and beta-galactosidase. Preferably the reporter protein is luciferase. In one embodiment, the reporter virus particles used in the method have the structural proteins of dengue serotype 1, dengue serotype 2, dengue serotype 3 or dengue serotype 4. Such reporter virus particles are available from commercial vendors or can be produced by the person skilled in the art. The reporter virus particles for one dengue serotype are mixed with the depleted sample for neutralization and incubated for a suitable period under suitable conditions, for example for 60 minutes at 36° C.

After neutralization the RVPs are added to permissive cells such as Raji cells, Vero cells, U937 cells or BHK cells expressing DC-SIGN and/or DC-SIGNR which enhances infection by enveloped viruses and the cells are cultured for a suitable period such as 72 hours. After the cell culturing the expression of the reporter protein such as luciferase is detected using methods known to the skilled person. The luciferase values are used to determine the EC50, i.e. the dilution of sera required for half-maximal neutralization of infection, by non-linear regression using a suitable software such as Prism 6 software. The EC50 of the depleted sample is compared with the EC50 of a control, i.e. non-depleted sample. Suitable RVP assay methods are described in Mukherjee et al. Methods Mol Biol. 2014; 1138:75-97; Ansarah-Sobrinho et al. Virology. 2008 Nov. 10; 381(1):67-74; Pierson et al. Virology. 2006 Mar. 1; 346(1):53-65; Dowd K A, DeMaso C R, Pierson T C. Genotypic Differences in Dengue Virus Neutralization Are Explained by a Single Amino Acid Mutation That Modulates Virus Breathing. mBio. 2015 Nov. 3; 6(6):e01559-15; VanBlargan et al., PLoS Pathog. 2013; 9(12):e1003761. The disclosures of which are herein incorporated by reference.

If depletion with one dengue serotype such as dengue-2 reduces the EC50 of the depleted sample in an RVP assay with another dengue serotype, e.g. dengue-3, below threshold, the antibodies are considered as cross-reactive. If depletion with one dengue serotype such as dengue-2 does not have an impact on the EC50 of the depleted sample in an RVP assay with another dengue serotype, e.g. dengue-3, the antibodies are considered as type-specific. Any result between reduction below threshold and no impact indicates that there is a mixture of cross-reactive and type-specific antibodies.

The method of (c) determines the level of antibodies against a non-structural protein 1 of dengue virus in the serum sample.

The term “non-structural protein” refers to those parts of dengue virus which do not form part of the viral envelope or capsid and which are necessary for viral replication. The non-structural proteins include nonstructural protein 1 (NS1), NS2A, NS2B, NS3, NS4A, NS4B and NS5. In the method used in the present invention preferably antibodies against NS1 are detected and quantified.

NS1 is the only viral protein secreted from DENV-infected cells and plays several roles in the viral lifecycle, including contributing to viral replication and immune evasion (Muller et al. (2013) Antiviral Res 98:192-208). NS1 is also a viral pathogenic factor that can act as a toxin, triggering the endothelial permeability and vascular leak that is a hallmark of severe dengue disease (Beatty et al. (2015) Sci Transl Med. 7(304):304ra141). DENV infection elicits NS1-specific antibodies (Shu et al. (2000) J Med Virol 62:224-32, Hertz et al. (2017) J Immunol 198:4025-35). No differences in anti-NS1 antibody titers have been observed between DF and DHF/DSS patients, however, antibodies to specific NS1 epitopes are higher in patients with less severe dengue (Lai et al. (2017) Sci Rep; 7: 6975). Further, vaccination with NS1 protects mice from lethal vascular leak, and passive transfer of NS1-specific serum abrogates NS1-induced lethality in vivo (Beatty et al. (2015) Sci Transl Med 7:304ra141). These data suggest that NS1-specific antibodies may contribute to protection against severe dengue disease.

In the present invention the level of antibodies against a non-structural protein, preferably against NS1, is detected and quantified by an ELISA (Enzyme-linked Immunosorbent Assay). ELISA is a solid phase assay based on the interaction between antigen and antibody which is detected by an antibody coupled to an enzyme capable of converting a chromogenic substrate to a chromogen. In the present invention preferably an indirect ELISA is used. In an indirect ELISA the first antibody binding to the antigen is not labelled, but a second antibody binding to the antibody which binds the antigen is used which second antibody is labelled to detect the interaction between the antigen and the first antibody.

In one embodiment, the ELISA comprises the following steps:
(i) providing a microplate coated with the non-structural protein or an antigenic fragment thereof, preferably with NS1 or an antigenic fragment thereof;
(ii) adding diluted serum samples to the coated microplate under conditions such that antibodies present in the serum samples can bind to the non-structural protein or a fragment thereof;
(iii) washing the microplate to remove unbound antibodies;
(iv) adding an enzyme-conjugated antibody capable of binding to the antibodies present in the serum samples under conditions such that the enzyme-conjugated antibody can bind to the antibodies present in the serum samples;
(v) washing the microplate to remove unbound antibodies;
(vi) adding the enzyme substrate under suitable conditions such that a color signal is produced; and
(vii) detecting and quantifying the color signal.

The non-structural protein, preferably NS1, with which the microplate is coated may be from any of the four dengue serotypes. Preferably, the microplate is coated with non-structural protein, preferably with NS1, from all four dengue serotypes, wherein each well of the microplate is coated with non-structural protein, preferably with NS1, of one dengue serotype.

The term “antigenic fragment” of the non-structural protein, preferably of NS1, means that not the full-length non-structural protein is used to coat the microplate, but only a shorter part of the non-structural protein, preferably of NS1, is used which is able to interact with an antibody binding to said non-structural protein, preferably NS1.

The washing steps of (iii) and (v) are preferably performed with PBS (phosphate-buffered saline) containing 0.1% Tween 20 (PBST).

The enzyme-conjugated antibody antibody capable of binding to the antibodies present in the serum samples is preferably an anti-IgG antibody which is capable of binding to all IgG antibodies present in a sample. Preferably, the enzyme-conjugated antibody is conjugated to a peroxidase or an alkaline phosphatase. Such enzyme-conjugated antibodies are commercially available for example from Sigma Aldrich.

The enzyme substrate is one which can be converted by the enzyme conjugated to the antibody to produce a detectable signal. If the enzyme conjugated to the antibody is a peroxidase, the substrate may be ABTS (2,2′-Azinobis [3-ethylbenzothiazoline-6-sulfonic acid), TMB (3,3′,5,5′-tetramethylbenzidine) or OPD (o-phenylene-diamine) Preferably, the substrate is ABTS. If the enzyme conjugated to the antibody is an alkaline phosphatase, the substrate may be p-Nitrophenyl Phosphate (PNPP).

Depending on the substrate used, different products are produced by reaction with the peroxidase. These products are detected and quantified on a microplate reader at a wavelength which is selected based on the substrate used and the detectable product produced by reacting the substrate with the enzyme.

A suitable ELISA assay to detect anti-NS1 antibodies in serum samples is described in Sharma et al. (2019) J. Infect. Dis. February 19. pii: jiz081. doi: 10.1093/infdis/jiz081.

The method of (d) determines the level of dengue-binding antibodies in the serum sample. The level of dengue-binding antibodies in the serum sample is preferably detected using an ELISA, more preferably using a sandwich ELISA. In a sandwich ELISA the microtiter plate is coated with a known amount of a capture antibody, before the antigen is applied. After binding of the antigen to the capture antibody, a second antibody or a sample containing a mixture of antibodies is added and then an enzyme-labelled antibody binding to the Fc region of the second antibody or of an antibody in the sample containing a mixture of antibodies is added.

In one embodiment the sandwich ELISA comprises the following steps:
(i) providing a microplate coated with a monoclonal antibody capable of binding to all dengue serotypes;
(ii) adding a live virion of a dengue serotype selected from the group consisting of serotypes 1, 2, 3 or 4 to the coated microplate under conditions such that the live virion of a dengue serotype selected from the group consisting of serotypes 1, 2, 3 or 4 can bind to the monoclonal antibody capable of binding to all dengue serotypes;
(iii) washing the microplate to remove unbound live virions;
(iv) adding diluted serum samples to the microplate under conditions such that antibodies present in the serum samples can bind to the live virion;
(v) washing the microplate to remove unbound antibodies;
(vi) adding an enzyme-conjugated antibody capable of binding to the antibodies present in the serum samples under conditions such that the enzyme-conjugated antibody can bind to the antibodies present in the serum samples; (vii) washing the microplate to remove unbound antibodies;
(viii) adding the enzyme substrate under suitable conditions such that a color signal is produced; and
(ix) detecting and quantifying the color signal.

A monoclonal antibody capable of binding to all dengue serotypes is an antibody which binds to an epitope which is present in all dengue serotypes. Suitable antibodies capable of binding to all dengue serotypes include 4G2 (Henchal et al. (1985) Am. J. Trop. Med. Hyg. 34: 162-169), 1M7 (Smith et al. (2014) J. Virol. 88: 12233-12241), All, B7, C8, C10 (Rouvinski et al. (2015) Nature 520: 109-113). Preferably, the monoclonal antibody capable of binding to all dengue serotypes is 4G2.

A “live virion of a dengue serotype” refers to an infectious virus having all the antigenic determinants of a native virus, i.e. the prM, E and C proteins of a dengue virus serotype. Preferably, the live virions of all dengue serotypes are added to different wells of a microplate so that in each well the interaction of one serotype with an antibody is investigated. The live virion of dengue serotype 1 is preferably of strain DENV-1 strain 16007, the live virion of dengue serotype 2 is preferably of DENV-2 strain 16681, the live virion of dengue serotype 3 is preferably of DENV-3 strain 16562 and the live virion of dengue serotype 4 is preferably of DENV-4 strain 1036.

The washing steps of (iii), (v) and (vii) are preferably performed with PBS (phosphate-buffered saline) containing 0.1% Tween 20 (PBST).

The conditions under which the live virion of a dengue serotype selected from the group consisting of serotypes 1, 2, 3 or 4 can bind to the monoclonal antibody capable of binding to all dengue serotypes, under which antibodies present in the serum samples can bind to the live virion and/or under which the enzyme-conjugated antibody can bind to the antibodies present in the serum samples are conditions which allow the binding and do not disturb the interaction between the binding partners. For example, the conditions are incubation in PBS with 0.1% Tween 20 (PBST).

The enzyme-conjugated antibody antibody capable of binding to the antibodies present in the serum samples is preferably an anti-IgG antibody which is capable of binding to all IgG antibodies present in a sample, but not to antibodies of other isotypes. Preferably, the enzyme-conjugated antibody is conjugated to a peroxidase or an alkaline phosphatase. Such enzyme-conjugated antibodies are commercially available for example from Sigma Aldrich.

The enzyme substrate is one which can be converted by the enzyme conjugated to the antibody to produce a detectable signal. If the enzyme conjugated to the antibody is a peroxidase, the substrate may be ABTS (2,2′-Azinobis [3-ethylbenzothiazoline-6-sulfonic acid), TMB (3,3′,5,5′-tetramethylbenzidine) or OPD (o-phenylene-diamine) Preferably, the substrate is ABTS. If the enzyme conjugated to the antibody is an alkaline phosphatase, the substrate may be p-Nitrophenyl Phosphate (PNPP).

Depending on the substrate used, different products are produced by reaction with the peroxidase. These products are detected and quantified on a microplate reader at a wavelength which is selected based on the substrate used.

A suitable ELISA assay to detect anti-dengue antibodies is described in Metz et al. (2018) Virol. J. 15:60 and in Dejnirattisai et al. (2010) Science 328: 745.

The method of (e) determines the presence and/or amount of flavivirus-reactive complement-fixing antibodies in a sample from a subject comprising the steps of:
Step 1: contacting an amount of a microsphere complex comprising a microsphere coupled to a flavivirus antigen with the sample to allow binding of the flavivirus-reactive complement-fixing antibodies in the sample to the flavivirus antigen;
Step 2: contacting an amount of complement component 1q (C1q) with the complement-fixing antibodies bound to the flavivirus antigen in step 1 to allow binding of the C1q to the heavy chain constant region of the complement-fixing antibodies;
Step 3: contacting an amount of a reporter antibody with the C1q bound to the complement-fixing antibodies in step 2 to allow binding of the reporter antibody to the C1q, wherein the reporter antibody binds to the C1q with the variable region of the reporter antibody and wherein the reporter antibody is attached to a detectable label; and
Step 4: detecting a signal from the reporter antibody bound to the C1q in step 3, wherein the signal is indicative for the presence and/or amount of the reporter antibody and wherein the presence and/or amount of the reporter antibody is indicative for the presence and/or amount of flavivirus-reactive complement-fixing antibodies in the sample.

According to one embodiment of the present invention, the method for determining the presence and/or amount of flavivirus-reactive complement-fixing antibodies in a sample from a subject comprises the further steps of:
Step 5: determining the presence and/or amount of the reporter antibody from the signal of step 4; and
Step 6: determining the presence and/or amount of flavivirus-reactive complement-fixing antibodies in the sample from the presence and/or amount of the reporter antibody determined in step 5.

According to one embodiment contacting in step 1 is carried out for about 30 to 90 minutes. In specific embodiments contacting in step 1 is carried out for about 60 minutes.

According to one embodiment contacting in step 2 is carried out for about 10 to 50 minutes. In specific embodiments contacting in step 2 is carried out for about 30 minutes.

According to one embodiment contacting in step 3.1 is carried out for about 10 to 50 minutes. In specific embodiments contacting in step 3.1 is carried out for about 30 minutes.

According to one embodiment contacting in step 3.2 is carried out for about 10 to 50 minutes. In specific embodiments contacting in step 3.2 is carried out for about 30 minutes.

In one specific embodiment contacting in step 1 is carried out for about 30 to 90 minutes, contacting in step 2 is carried out for about 10 to 50 minutes, contacting in step 3.1 is carried out for about 10 to 50 minutes, and contacting in step 3.2 is carried out for about 10 to 50 minutes.

In a more specific embodiment contacting in step 1 is carried out for about 60 minutes, contacting in step 2 is carried out for about 30 minutes, contacting in step 3.1 is carried out for about 30 minutes, and contacting in step 3.2 is carried out for about 30 minutes.

In one embodiment of the present invention, the detectable label to which the reporter antibody is attached to is a fluorescence label selected from the group consisting of xanthene, fluorescein isothiocyanate, rhodamine, phycoerythrin, cyanine, coumarin, and any derivative thereof. In a preferred embodiment the detectable label is phycoerythrin.

In one embodiment the flavivirus is selected from the group consisting of dengue virus, zika virus, West Nile virus, Japanese encephalitis virus, Tick-Borne encephalitis virus, Yellow Fever virus, Murray Valley encephalitis virus, and St. Louis encephalitis virus.

In other embodiments the antigen is selected from the group consisting of virus like particle (VLP), non-structural protein 1, envelope protein, pre-membrane protein, membrane protein, capsid protein, non-structural protein 2A, non-structural protein 2B, non-structural protein 3, non-structural protein 4A, non-structural protein 4B, and non-structural protein 5 and any derivative thereof.

In specific embodiments the flavivirus antigen is a DENV VLP. The DENV VLP can be of any serotype (DENV1-4). In other specific embodiments the flavivirus antigen is DENV NS1. The DENV NS1 can be of any serotype (DENV1-4).

In other specific embodiments the flavivirus antigen is a ZIKV VLP. In other specific embodiments the flavivirus antigen is ZIKV NS1.

In certain embodiments, the C1q is present within a complement-component serum, which may be human complement-component serum. In certain embodiments, the C1q is purified from plasma. Purification can be carried out by any protein purification method known in the art, such as filtration, centrifugation, chromatographic separation, or buffer exchange. In other embodiments, the C1q is of human origin. In specific embodiments the C1q is purified human C1q. In general, the origin of the C1q is dependent on the origin of complement-fixing Abs in the sample. For instance, for detection of human complement-fixing Abs, human C1q is suitable.

In certain embodiments, the sample is heat-inactivated. Heat-inactivation can be carried out at about 56° C. for about 30 minutes. In general, heat-inactivation can be varied according to the type of sample to be analyzed.

In other embodiments the sample is a blood sample, in particular a plasma or serum sample. In general, the method can be carried out with either matrices i.e. serum or plasma. Preferably, the sample was obtained from an individual immunized with a Dengue vaccine. Alternatively, the sample may have been obtained from a patient suffering from Dengue disease.

In other embodiments the sample is a urine sample.

In specific embodiments, the sample is a heat-inactivated serum or plasma sample.

In one embodiment the subject is selected from the group consisting of mouse, primate, non-human primate, human, rabbit, cat, rat, horse, and sheep. In preferred embodiments the subject is a non-human primate. In more preferred embodiments the subject is human.

In other embodiments the subject is seronegative for the flavivirus. In other embodiments the subject is seropositive for the flavivirus. In specific embodiments the subject is seronegative for DENV. In other specific embodiments the subject is seropositive for DENV.

In some embodiments of the present invention the subject is vaccinated with a flavivirus vaccine.

In one embodiment of the present invention, the signal from the reporter antibody detected in step 4 is resulting from the detectable label to which the reporter antibody is attached. In a specific embodiment, the signal in step 4 is a fluorescence signal. In even more specific embodiments, the signal in step 4 is a fluorescence signal resulting from phycoerythrin. The signal in step 4 can be detected upon irradiation with a light source as present in any suitable detection system.

Within the meaning of the invention, in embodiments wherein no pre-reporter Ab is applied, the signal from the reporter antibody in step 4 is resulting from a reporter antibody which is bound to C1q, wherein C1q is bound to the complement-fixing Abs bound to the flavivirus antigen coupled to microspheres in a microsphere complex.

Within the meaning of the invention, in embodiments wherein a pre-reporter Ab is applied, the signal from the reporter antibody in step 4 is resulting from a reporter antibody which is bound to the pre-reporter Ab, wherein the pre-reporter Ab is bound to C1q, wherein C1q is bound to the complement-fixing Abs bound to the flavivirus antigen coupled to microspheres in a microsphere complex.

In specific embodiments of the present invention the presence and/or amount of reporter Ab in step 5 is determined by comparing the signal of step 4 to a standard curve, wherein the standard curve comprises signals resulting from known amounts of reporter Ab.

In specific embodiments of the invention the presence and/or amount of flavivirus-reactive complement-fixing Abs in the sample in step 6 is determined based on the amount of the reporter Ab determined in step 5 as the amount of reporter Ab is in direct proportion to the complement-fixing Abs present within a sample.

In one embodiment, the method for characterizing the immune response of a subject to a tetravalent dengue virus composition administered to said subject comprises performing with a blood serum sample from said subject the method of the first, second or third aspect and a method to determine the level of neutralizing antibodies in said sample.

In one embodiment, the method for characterizing the immune response of a subject to a tetravalent dengue virus composition administered to said subject comprises performing with a blood serum sample from said subject the method of the first, second or third aspect and a method comprising depleting antibodies against one dengue serotype from said sample followed by determining in the depleted sample the level of neutralizing antibodies against at least one serotype different from the dengue serotype used for depleting the antibodies, to detect the presence of type-specific and cross-reactive neutralizing antibodies in said sample.

In one embodiment, the method for characterizing the immune response of a subject to a tetravalent dengue virus composition administered to said subject comprises performing with a blood serum sample from said subject the method of the first, second or third aspect and a method to quantitate the level of antibodies against a non-structural protein 1 of dengue virus in said sample.

In one embodiment, the method for characterizing the immune response of a subject to a tetravalent dengue virus composition administered to said subject comprises performing with a blood serum sample from said subject the method of the first, second or third aspect and a method to quantitate the level of dengue-binding antibodies in said sample.

In one embodiment, the method for characterizing the immune response of a subject to a tetravalent dengue virus composition administered to said subject comprises performing with a blood serum sample from said subject the method of the first, second or third aspect and a method to determine the presence and/or amount of flavivirus-reactive complement-fixing antibodies in said sample.

In one embodiment, the method for characterizing the immune response of a subject to a tetravalent dengue virus composition administered to said subject comprises performing with a blood serum sample from said subject:
(a) the method of the first, second or third aspect;
(b) a method to determine the level of neutralizing antibodies in said sample; and
(c) a method comprising depleting antibodies against one dengue serotype from said sample followed by determining in the depleted sample the level of neutralizing antibodies against at least one serotype different from the dengue serotype used for depleting the antibodies, to detect the presence of type-specific and cross-reactive neutralizing antibodies in said sample.

In one embodiment, the method for characterizing the immune response of a subject to a tetravalent dengue virus composition administered to said subject comprises performing with a blood serum sample from said subject:
(a) the method of the first, second or third aspect;
(b) a method to determine the level of neutralizing antibodies in said sample; and
(c) a method to quantitate the level of antibodies against a non-structural protein 1 of dengue virus in said sample

In one embodiment, the method for characterizing the immune response of a subject to a tetravalent dengue virus composition administered to said subject comprises performing with a blood serum sample from said subject:
(a) the method of the first, second or third aspect;
(b) a method to determine the level of neutralizing antibodies in said sample; and
(c) a method to quantitate the level of dengue-binding antibodies in said sample.

In one embodiment, the method for characterizing the immune response of a subject to a tetravalent dengue virus composition administered to said subject comprises performing with a blood serum sample from said subject:
(a) the method of the first, second or third aspect;
(b) a method comprising depleting antibodies against one dengue serotype from said sample followed by determining in the depleted sample the level of neutralizing antibodies against at least one serotype different from the dengue serotype used for depleting the antibodies, to detect the presence of type-specific and cross-reactive neutralizing antibodies in said sample; and
(c) a method to quantitate the level of antibodies against a non-structural protein 1 of dengue virus in said sample.

In one embodiment, the method for characterizing the immune response of a subject to a tetravalent dengue virus composition administered to said subject comprises performing with a blood serum sample from said subject:
(a) the method of the first, second or third aspect;
(b) a method comprising depleting antibodies against one dengue serotype from said sample followed by determining in the depleted sample the level of neutralizing antibodies against at least one serotype different from the dengue serotype used for depleting the antibodies, to detect the presence of type-specific and cross-reactive neutralizing antibodies in said sample; and
(c) a method to quantitate the level of dengue-binding antibodies in said sample.

In one embodiment, the method for characterizing the immune response of a subject to a tetravalent dengue virus composition administered to said subject comprises performing with a blood serum sample from said subject:
(a) the method of the first, second or third aspect;
(b) a method to quantitate the level of dengue-binding antibodies in said sample; and
(c) a method to quantitate the level of antibodies against a non-structural protein 1 of dengue virus in said sample.

In one embodiment, the method for characterizing the immune response of a subject to a tetravalent dengue virus composition administered to said subject comprises performing with a blood serum sample from said subject:
(a) the method of the first, second or third aspect;
(b) a method to determine the level of neutralizing antibodies in said sample;
(c) a method comprising depleting antibodies against one dengue serotype from said sample followed by determining in the depleted sample the level of neutralizing antibodies against at least one serotype different from the dengue serotype used for depleting the antibodies, to detect the presence of type-specific and cross-reactive neutralizing antibodies in said sample; and
(d) a method to quantitate the level of antibodies against a non-structural protein 1 of dengue virus in said sample.

In one embodiment, the method for characterizing the immune response of a subject to a tetravalent dengue virus composition administered to said subject comprises performing with a blood serum sample from said subject:
(a) the method of the first, second or third aspect;
(b) a method to determine the level of neutralizing antibodies in said sample;
(c) a method comprising depleting antibodies against one dengue serotype from said sample followed by determining in the depleted sample the level of neutralizing antibodies against at least one serotype different from the dengue serotype used for depleting the antibodies, to detect the presence of type-specific and cross-reactive neutralizing antibodies in said sample; and
(d) a method to quantitate the level of dengue-binding antibodies in said sample.

In one embodiment, the method for characterizing the immune response of a subject to a tetravalent dengue virus composition administered to said subject comprises performing with a blood serum sample from said subject:
(a) the method of the first, second or third aspect;
(b) a method to determine the level of neutralizing antibodies in said sample;
(c) a method to quantitate the level of antibodies against a non-structural protein 1 of dengue virus in said sample; and
(d) a method to quantitate the level of dengue-binding antibodies in said sample.

In one embodiment, the method for characterizing the immune response of a subject to a tetravalent dengue virus composition administered to said subject comprises performing with a blood serum sample from said subject:
(a) the method of the first, second or third aspect;
(b) a method comprising depleting antibodies against one dengue serotype from said sample followed by determining in the depleted sample the level of neutralizing antibodies against at least one serotype different from the dengue serotype used for depleting the antibodies, to detect the presence of type-specific and cross-reactive neutralizing antibodies in said sample;
(c) a method to quantitate the level of antibodies against a non-structural protein 1 of dengue virus in said sample; and
(d) a method to quantitate the level of dengue-binding antibodies in said sample.

In one embodiment, the method for characterizing the immune response of a subject to a tetravalent dengue virus composition administered to said subject comprises performing with a blood serum sample from said subject:
(a) the method of the first, second or third aspect;
(b) a method to determine the level of neutralizing antibodies in said sample;
(c) a method comprising depleting antibodies against one dengue serotype from said sample followed by determining in the depleted sample the level of neutralizing antibodies against at least one serotype different from the dengue serotype used for depleting the antibodies, to detect the presence of type-specific and cross-reactive neutralizing antibodies in said sample;
(d) a method to quantitate the level of antibodies against a non-structural protein 1 of dengue virus in said sample; and
(e) a method to quantitate the level of dengue-binding antibodies in said sample.

In one embodiment, the method for characterizing the immune response of a subject to a tetravalent dengue virus composition administered to said subject comprises performing with a blood serum sample from said subject:
(a) the method of the first, second or third aspect;
(b) a method to determine the level of neutralizing antibodies in said sample;
(c) a method comprising depleting antibodies against one dengue serotype from said sample followed by determining in the depleted sample the level of neutralizing antibodies against at least one serotype different from the dengue serotype used for depleting the antibodies, to detect the presence of type-specific and cross-reactive neutralizing antibodies in said sample;
(d) a method to quantitate the level of antibodies against a non-structural protein 1 of dengue virus in said sample;
(e) a method to quantitate the level of dengue-binding antibodies in said sample; and
(f) a method to determine the presence and/or amount of flavivirus-reactive complement-fixing antibodies in said sample.

The present invention further provides a method for characterizing the immune response of a subject to a virus-containing vaccine composition administered to said subject, comprising performing with a serum sample from said subject at least two of methods selected from the group consisting of:
(a) a method to determine the level of neutralizing antibodies in said sample;
(b) a method comprising depleting antibodies against one dengue serotype from said sample followed by determining in the depleted sample the level of neutralizing antibodies against at least one serotype different from the dengue serotype used for depleting the antibodies, to detect the presence of type-specific and cross-reactive neutralizing antibodies in said sample;
(c) a method to determine the level of antibodies against a non-structural protein 1 of dengue virus in said sample;
(d) a method to determine the level of dengue-binding antibodies in said sample;
(e) a method to determine the presence and/or amount of flavivirus-reactive complement-fixing antibodies in said sample; and
(f) the avidity assay method according to the present invention.

In a preferred embodiment the virus-containing vaccine composition is a tetravalent dengue vaccine composition as described above.

In the subsequent paragraphs the following abbreviations are used:
“(a)” for a method to determine the level of neutralizing antibodies in said sample
“(b)” for a method comprising depleting antibodies against one dengue serotype from said sample followed by determining in the depleted sample the level of neutralizing antibodies against at least one serotype different from the dengue serotype used for depleting the antibodies, to detect the presence of type-specific and cross-reactive neutralizing antibodies in said sample
“(c)” for a method to determine the level of antibodies against a non-structural protein 1 of dengue virus in said sample
“(d)” for a method to determine the level of dengue-binding antibodies in said sample
“(e)” for a method to determine the presence and/or amount of flavivirus-reactive complement-fixing antibodies in said sample
“(f)” for the avidity assay method according to the present invention

In a preferred embodiment the method for characterizing the immune response comprises or consists of the following methods: (a)(b), (a)(c), (a)(d), (a)(e), (a)(f), (b)(c), (b)(d), (b)(e), (b)(f), (c)(d), (c)(f), (d)(e), (d)(f) and (e)(f).

In a further preferred embodiment the method for characterizing the immune response comprises or consists of the following methods: (a)(b)(c), (a)(b)(d), (a)(b)(e), (a)(b)(f), (a)(c)(d), (a)(c)(e), (a)(c)(f), (a)(d)(e), (a)(d)(f), (a)(e)(f), (b)(c)(d), (b)(c)(e), (b)(c)(f), (b)(d)(f), (b)(e)(f), (c)(d)(e), (c)(d)(f), (c)(e)(f) and (d)(e)(f).

In a further preferred embodiment the method for characterizing the immune response comprises or consists of the following methods: (a)(b)(c)(d), (a)(b)(c)(e), (a)(b)(c)(f), (a)(b)(d)(e), (a)(b)(d)(f), (a)(b)(e)(f), (a)(c)(d)(e), (a)(c)(d)(f), (a)(d)(e)(f), (b)(c)(d)(e), (b)(c)(d)(f), (b)(c)(e)(f), (b)(c)(e)(f), (b)(d)(e)(f) and (c)(d)(e)(f).

In a further preferred embodiment the method for characterizing the immune response comprises or consists of the following methods: (a)(b)(c)(d)(e), (a)(b)(c)(d)(f), (a)(b)(c)(e)(f), (a)(b)(d)(e)(f), (a)(c)(d)(e)(f) and (b)(c)(d)(e)(f)

In a further preferred embodiment the method for characterizing the immune response comprises or consists of the following method: (a)(b)(c)(d)(e)(f).

In a further aspect the present invention provides a method for predicting the protective efficacy of a dengue vaccine candidate comprising
determining the presence and/or amount of at least two immune response parameters selected from the group consisting of serotype specific neutralizing antibodies, cross-reactive neutralizing antibodies, complement-fixing antibodies, dengue total binding IgG response and high affinity antibodies against dengue antigens in a blood sample from a subject vaccinated with the dengue vaccine candidate, and
predicting the dengue vaccine candidate to provide protective efficacy if the presence of at least two immune response parameters selected from the group consisting of serotype specific neutralizing antibodies, cross-reactive neutralizing antibodies, dengue reactive complement-fixing antibodies, dengue total binding IgG response and high affinity antibodies against dengue antigens is determined in the blood sample.

A goal for DENV vaccines is to elicit potent antibody responses capable of conferring durable protection against diverse global DENV strains.

Neutralizing Antibodies

Multiple lines of evidence demonstrate that the presence or magnitude of neutralizing antibodies (NAbs) directed against the dengue envelope protein is associated with reduced frequency and/or severity of infection with DENV or other flaviviruses. The present inventors have successfully characterized the neutralizing antibodies induced by TDV, including type-specific and cross-reactive NAbs and neutralization of diverse DENV genotypes

Antibodies Against Nonstructural Proteins

DENV infection elicits NS1-specific antibodies. No differences in anti-NS1 antibody titers have been observed between patients with dengue fever and dengue hemorrhagic fever/dengue shock syndrome, however, antibodies to specific NS1 epitopes are higher in patients with less severe dengue. Further, vaccination with NS1 protects mice from lethal vascular leak and passive transfer of NS1-specific serum abrogates NS1-induced lethality in vivo. These data suggest that NS1-specific antibodies may contribute to protection against severe dengue disease. However, the role of DENV NS1-specific immunity in protection mediated by vaccination in humans has not been investigated.

The present inventors found that TDV can elicit functional immune responses to DENV non-structural proteins in both children and adults. Vaccination elicits DENV-2 NS1-specific antibodies that are cross-reactive with NS1 from DENV-1, DENV-3 and DENV-4.

Binding Antibodies

DENV infection elicits DENV-specific binding antibodies that includes neutralizing and non-neutralizing binding antibodies with antiviral effector functions.

The present inventors developed a DENV-specific IgG antigen capture ELISA assay to quantitate total binding antibodies to live DENV virions. This assay is different from the iELISA, which uses mixed DENV antigens of all four serotypes prepared by acetone fixation of antigen derived from mouse brain and measures titers of antibody that compete with pooled, ammonium sulfate precipitated DENV convalescent sera. Instead, live DENV virion antigens were selected which retain quaternary epitopes that are targets of NAbs and used a direct method to avoid the inherent lot to lot variability of pooled convalescent serum as a reagent.

The present inventors found that vaccination with TDV significantly increased the total binding IgG responses to the different DENV virions.

Complement-Fixing Antibodies

The complement system is an arm of the innate immune response that enhances the functionality of antibodies, contributing to opsonization, killing and clearance of pathogens. Formation of an antigen-antibody immunocomplex, e.g. on a pathogen surface, leads to binding of C1q, a key molecule capable of initiating the activation of the classical pathway of the complement system upon interaction with certain IgG subclasses and IgM. As a result of interaction between C1q and immunocomplexes, complement components are deposited (fixed) on the surface of the virus particle. Complement fixation promotes complement-mediated lysis of virus particles and infected cells, tags virus particles bound to antibodies for clearance by macrophages, dendritic cells, platelets, red blood cells and B cells through complement receptors, and promotes B cell activation.

The complement system has been demonstrated to contribute to protection against flaviviruses.

As outlined in the examples, it could be demonstrated that TDV successfully induced complement-fixing antibodies against DENV antigens.

Antibody Affinity Maturation and High Avidity Antibodies

After antigen exposure, antibody somatic hypermutation takes place in germinal centers. Germinal center B cells express enzymes which introduce point mutations in the Ig heavy and light chain genes. The resulting B cell repertoire is then selected and enriched for high antibody affinity for the target antigen. Iterative rounds of selection and proliferation results in a population of antibodies that are enriched for higher affinity binders, based on successive accumulation of somatic mutations over time. The process of antibody affinity maturation forms the basis for evolution of effective antibody responses to specific pathogens from the diverse B cell repertoire. Collectively, antibodies with increased affinities after antigen exposure contribute to an overall increase in polyclonal antibody avidity.

The importance of antibody affinity maturation to effective antiviral responses is well established. For example, HIV antibody affinity correlates with neutralization potency and breadth. Affinity maturation of B cells specific for conserved epitopes after sequential exposure to infection is required for protection from re-infection by diverse influenza viruses and is required to generate monoclonal antibodies of sufficient potency for Ebola virus therapy. Repeated DENV infections have been shown to increase monoclonal and polyclonal antibody avidity and increased neutralization potency.

The present inventors have successfully demonstrated that TDV induced affinity-matured antibodies of high avidity.

Based on the fact that TDV has been demonstrated in clinical trials such as DEN-301 as an effective vaccine against dengue, the present inventors could determine which immune response parameters are relevant for the prediction of protective efficacy of a dengue vaccine on the basis of assessment of immune responses that are associated with protection from dengue.

In a preferred embodiment, the at least two immune response parameters are selected from the group consisting of
(i) serotype-specific neutralizing antibodies against at least one dengue structural protein,
(ii) cross-reactive neutralizing antibodies against at least one dengue structural protein,
(iii) cross-reactive antibodies against at least one dengue non-structural protein,
(iv) complement-fixing antibodies against at least one dengue structural protein,
(v) dengue total binding IgG response, and
(vi) high affinity antibodies against dengue antigens.

Preferably the at least two immune response parameters are selected from the group consisting of:
(i) neutralizing antibodies against at least one dengue serotype
(ii) serotype-specific neutralizing antibodies against at least one dengue serotype,
(ii) cross-reactive neutralizing antibodies against at least one dengue serotype,
(iii) antibodies against dengue non-structural protein 1 of at least one dengue serotype,
(iv) complement-fixing antibodies against at least one dengue serotype,
(v) dengue total binding IgG response against at least one dengue serotype, and
(vi) high affinity antibodies against dengue antigens from at least one dengue serotype.

In a more preferred embodiment of this method, the dengue structural protein is dengue E protein and/or the dengue non-structural protein is dengue NS1 protein.

In a further preferred embodiment the above method for predicting the protective efficacy of the dengue vaccine candidate is performed using the above described methods for characterizing an immune response.

The “dengue total binding IgG response” may be determined by an ELISA method or fluorescent method as known to the person skilled in the art. The secondary antibody used in these assays may be a pan anti-IgG antibody reactive with IgG1, IgG2, IgG3 and IgG4 subtypes.

“high affinity antibodies against dengue antigens” in accordance with the present invention may be determined by the avidity assay as described herein. Further, the affinity may also be determined by a conventional ELISA or fluorescent binding assay known to the person skilled in the art. An antibody is considered a high affinity antibody if it exhibits an affinity of at least 500 as Avidity index, preferably at least 1000 as Avidity index, most preferred of at least 5000 as Avidity index.

In a preferred embodiment the method for predicting the protective efficacy comprises or consists of the following immune response parameters: (i)(ii), (i)(iii), (i)(iv), (i)(v), (i)(vi), (ii)(iii), (ii)(iv), (ii)(v), (ii)(vi), (iii)(iv), (iii)(vi), (iv)(v), (iv)(vi) and (v)(vi).

In a further preferred embodiment the method for predicting the protective efficacy comprises or consists of the following immune response parameters: (i)(ii)(iii), (i)(ii)(iv), (i)(ii)(v), (i)(ii)(vi), (i)(iii)(iv), (i)(iii)(v), (i)(iii)(vi), (i)(iv)(v), (i)(iv)(vi), (i)(v)(vi), (ii)(iii)(iv), (ii)(iii)(v), (ii)(iii)(vi), (ii)(iv)(vi), (ii)(v)(vi), (iii)(iv)(v), (iii)(iv)(vi), (iii)(v)(vi) and (iv)(v)(vi).

In a further preferred embodiment the method for predicting the protective efficacy comprises or consists of the following immune response parameters: (i)(ii)(iii)(iv), (i)(ii)(iii)(v), (i)(ii)(iii)(vi), (i)(ii)(iv)(v), (i)(ii)(iv)(vi), (i)(ii)(v)(vi), (i)(iii)(iv)(v), (i)(iii)(iv)(vi), (i)(iv)(v)(vi), (ii)(iii)(iv)(v), (ii)(iii)(iv)(vi), (ii)(iii)(v)(vi), (ii)(iii)(v)(vi), (ii)(iv)(v)(vi) and (iii)(iv)(v)(vi).

In a further preferred embodiment the method for predicting the protective efficacy comprises or consists of the following immune response parameters: (i)(ii)(iii)(iv)(v), (i)(ii)(iii)(iv)(vi), (i)(ii)(iii)(v)(vi), (i)(ii)(iv)(v)(vi), (i)(iii)(iv)(v)(vi) and (ii)(iii)(iv)(v)(vi).

In a further preferred embodiment the method for predicting the protective efficacy comprises or consists of the following immune response parameters:
(i)(ii)(iii)(iv)(v)(vi).

In a further aspect the present invention provides a method for preparing a vaccine formulation comprising
performing the method for predicting the protective efficacy of a dengue vaccine candidate according to the present invention; and
formulating the vaccine candidate predicted to provide protective efficacy with a pharmaceutically acceptable excipient.

Pharmaceutically acceptable excipients and methods for formulation are known to the person skilled in the art. Preferably, the formulation is for parenteral administration. More preferably, the formulation is for intravenous, intramuscular or subcutaneous administration. Suitable pharmaceutically acceptable excipients include, without limitation, water, saline, buffered saline, phosphate buffer, alcohol/aqueous solutions, emulsions or suspensions. Other conventionally employed diluents and excipients may be added in accordance with conventional techniques. Such carriers can include ethanol, polyols, and suitable mixtures thereof, vegetable oils, and injectable organic esters. Buffers and pH adjusting agents may also be employed. Buffers include, without limitation, salts prepared from an organic acid or base. Representative buffers include, without limitation, organic acid salts, such as salts of citric acid, e.g., citrates, ascorbic acid, gluconic acid, carbonic acid, tartaric acid, succinic acid, acetic acid, orphthalic acid, Tris, trimethanmine hydrochloride, or phosphate buffers. Parenteral carriers can include sodium chloride solution, Ringer's dextrose, dextrose, trehalose, sucrose, and sodium chloride, lactated Ringer's or fixed oils. Intravenous carriers can include fluid and nutrient replenishers, electrolyte replenishers, such as those based on Ringer's dextrose and the like. Preservatives and other additives such as, for example, antimicrobials, antioxidants, chelating agents (e.g. EDTA), inert gases and the like may also be provided in the pharmaceutical carriers. The present invention is not limited by the selection of the carrier. The preparation of these pharmaceutically acceptable compositions, from the above-described components, having appropriate pH isotonicity, stability and other conventional characteristics is within the skill of the art.

The vaccine candidate predicted to provide protective efficacy is present in the final formulation in an amount sufficient for inducing an immune response in a subject.

In a further aspect the present invention provides a vaccine formulation obtainable by the method described herein.

The methods referred to in the above embodiments are as described in detail herein.

### EXAMPLES

The following Examples are included to demonstrate certain aspects and embodiments of the invention as described in the claims. It should be appreciated by those of skill in the art, however, that the following description is illustrative only and should not be taken in any way as a restriction of the invention.

### Example 1: Reactivity of Avidity Assay Using Antibodies from TDV-Vaccinated Healthy Individuals

Materials and Methods

Sample and Reagents

8 vaccinated subjects with no anti-dengue virus-specific response before vaccination have been selected from the DEN-203 clinical trial (ClinicalTrials.gov identifier NCT01511250), i.e. a phase 2 clinical trial from Puerto Rico, Colombia, Singapore, and Thailand and sera from days 0, 28, 90, 120, 180 and 360 days post vaccination were obtained from these subjects. The sera were stored at −80° C. until use and thawed at 4° C. storage overnight before purification. Dengue Virus Like particles were purchased from Native antigen; Dengue 1 strain: Nauru/Western Pacific/1974 (UniProtKB/Swiss-Prot: P17763.2), Dengue 2 strain: Thailand/16681/84 (UniProtKB/Swiss-Prot: P29990.1), Dengue 3 strain: Sri Lanka/1266/2000 (UniProtKB/Swiss-Prot: Q6YMS4.1) and Dengue 4 strain: Dominica/814669/1981 (UniProtKB/Swiss-Prot: P09866.2). 20% of Envelope protein, E-protein, were replaced by the corresponding sequence of Japanese encephalitis strain SA-14 (UniProtKB/Swiss-Prot: P27395.1), amino acid sequence 397-495. SA-Biosensor, biosensor coated with Streptavidin, was purchased from Forte Bio.

Antibody Purification

IgG were purified from 200 μL of sera by Protein G Sepharose (GE). Briefly, 200 μL of sera were mixed with 3 mL Dulbecco's Phosphate-buffered saline (D-PBS) and 0.6 mL 50% Protein G Sepharose in a 15 mL centrifuge tube. These centrifuged tubes were mixed for 90 min at room temperature with a shaker. After centrifugation, Protein G Sepharose slurry was transferred to 24 well Unifilter (GE). The slurry was washed with D-PBS 4 times and eluted with 0.1M Glycine HCl pH2.7 for 4 times. The eluates were immediately neutralized to pH 7.0 to 7.5 with 1M Tris HCl pH8.0. The solution was buffer-exchanged with Amicon Ultra 4 (Millipore MWCO 30 KDa). The absorption at 280 nm in these antibody solutions was measured by NanoDrop 2000 (Thermo) and the IgG concentration was calculated. Then these samples were diluted with D-PBS to 2.5 mg/mL for each sample ID. The antibody purity was confirmed by SDS PAGE (NuPAGE 4-12% Bis-Tris Gel, Thermo). 2 ng protein sample was reduced at 70° C. for 10 min and applied to the gel. These gels were stained by simple stain blue (Thermo) and these images were captured by ChemoDoc Touch imaging system (Bio Rad) and calculated purity was confirmed more than 80% from both IgL and IgH band by Image Lab software (Bio-Rad).

Optimization of Biotinylation of Dengue VLP

Biotinylation of Dengue VLP was optimized using 20, 50 and 100 excess mole EZ-Link sulfo-NHS-Biotin. 80 μg of DENV1, 2, 3 and 4 VLPs were reacted for 60 min at room temperature respectively. After biotinylation, the excess biotinylation reagents were removed and the biotinylated VLPs were buffer-exchanged with D-PBS using Amicon Ultra 4 (Millipore MWCO 30 KDa). Biotinylation was evaluated by Octet Avidity assay using purified IgG from DEN203 sample 1053005 at 90 Days sera.

Preparation of Biotinylated Dengue VLP

For DEN203 avidity assays, 200 μg of Dengue Virus Like Particle, VLP, type 1, 2, 3 and 4 (Native Antigen) were biotinylated with 50 excess moles of EZ-Link Sulfo-NHS-Biotin (Thermo) following the manufacturer's protocol. The reaction took place at room temperature for 60 min, after the reaction, the excess biotinylation reagents were removed and these biotinylated VLPs buffer-exchanged with D-PBS using Amicon Ultra 4 (Millipore MWCO 30 kDa). The biotinylated Dengue VLP solutions were adjusted to a concentration of 0.4 mg/mL and were stored at −80° C. until use.

Avidity Assay

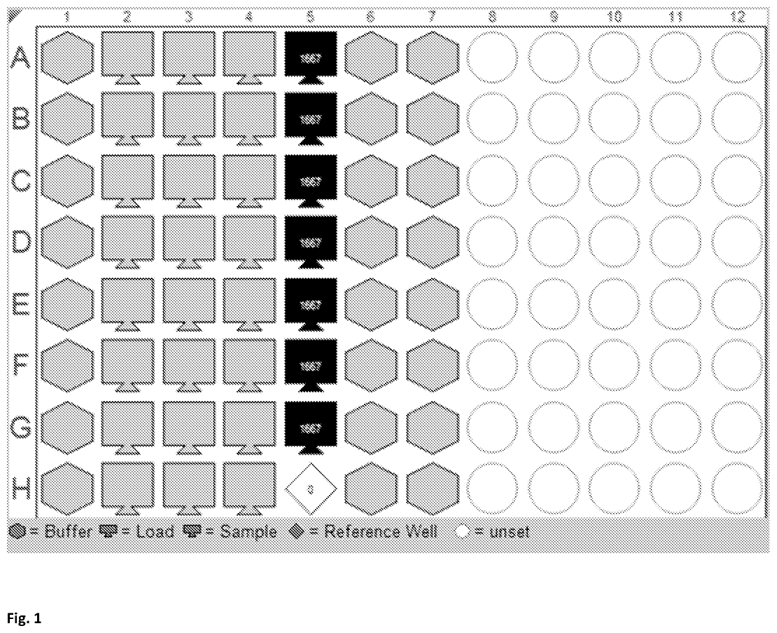

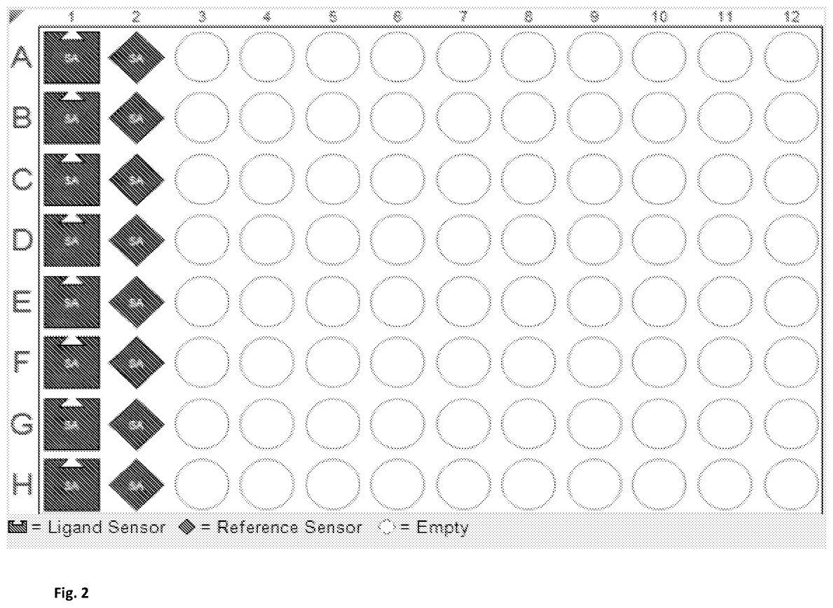

Avidity was measured by Octet 96 red (Forte Bio) using SA biosensor (Forte Bio). Briefly, SA biosensors were hydrated with D-PBS at least 10 min before an analysis. 5 ug/mL biotinylated Dengue VLP, in 0.1% BSA phosphate-buffered saline with Tween-20 (PBS-T) was captured on the SA biosensor and then the excess streptavidin on the surface was blocked with 50 μg/mL Biocytin (Thermo). 250 ug/mL anti-DENV polyclonal antibodies purified from DEN203 patients' sera in 0.1% BSA PBS-T were bound to the biosensor for 1800 sec and then the sensors were incubated in 0.1% BSA PBS-T 0.35M NaCl for 1200 sec to dissociate the binding antibody. These reactions were conducted at 30° C. and assay plates were shaken at 1000 rpm. (FIGS. 1 and 2, Tables 1 and 2).

TABLE 1
Example of plate layout

| Well | Well | | | Conc. |
|---|---|---|---|---|
| Location | Type | Sample ID | Description | ug/mL |
| A1-H1 | Buffer | 0.1% BSA PBST | | |
| A2-H2 | Load | Biotin DENV3 VLP | 0.1% BSA PBST | |
| | | 5 ug/mL | | |
| A3-H3 | Load | 0.1% BSA PBST | | |
| A4-H4 | Load | 50 ug/mL Biocytin | 0.1% BSA PBST | |
| A5 | Sample | 1053010 0 day | 0.1% BSA PBST | 250 |
| B5 | Sample | 1053010 28 day | 0.1% BSA PBST | 250 |
| C5 | Sample | 1053010 90 day | 0.1% BSA PBST | 250 |
| D5 | Sample | 1053010 120 day | 0.1% BSA PBST | 250 |
| E5 | Sample | 1053010 180 day | 0.1% BSA PBST | 250 |
| F5 | Sample | 1053010 360 day | 0.1% BSA PBST | 250 |
| G5 | Sample | PC sera IgG 250 | 0.1% BSA PBST | 250 |
| | | ug/mL | | |
| H5 | Reference | no AB | 0.1% BSA PBST | 0 |
| | Well | | | |
| A6-H6 | Buffer | 0.1% BSA PBST | | |
| A7-H7 | Buffer | 0.1% BSA PBST + | | |
| | | 0.35M NaCl | | |

TABLE 2
Assay conditions
Assay program

| | | | | | Assay |
|---|---|---|---|---|---|
| Assay Step | Step Data | Sensor | Sample | | Time |
| Number | Name | Column | Column | Step Type | sec |
| 1 | Sensor Check | 1 | 1 | Baseline | 30 |
| 2 | Loading | 1 | 2 | Loading | 600 |
| 3 | Blocking | 1 | 4 | Loading | 200 |
| | Biocytin | | | | |
| 4 | Baseline | 1 | 6 | Baseline | 180 |
| 5 | Association | 1 | 5 | Association | 1800 |
| | 1800 | | | | |
| 6 | Dissociation | 1 | 7 | Dissociation | 1200 |
| | 1200 | | | | |
| 7 | Sensor Check | 2 | 1 | Baseline | 30 |
| 8 | Loading | 2 | 3 | Loading | 600 |
| 9 | Blocking | 2 | 4 | Loading | 200 |
| | Biocytin | | | | |
| 10 | Baseline | 2 | 6 | Baseline | 180 |
| 11 | Association | 2 | 5 | Association | 1800 |
| | 1800 | | | | |
| 12 | Dissociation | 2 | 7 | Dissociation | 1200 |
| | 1200 | | | | |

For the negative subtractions, a double subtraction protocol was applied with a combination of antibody/DENV VLP; antibody/no DENV VLP; no antibody/DENV VLP; and no antibody/no DENV VLP to assess the dissociation rate precisely. Data analysis was conducted by Octet Data Analysis Software (Forte Bio, version 9.0.0.10). For evaluation, two parameters for antibody avidity have been assessed. The response, correlated with the anti-DENV antibody concentration, was measured by the response values at 1800 sec association time. k_{off}, the antibody dissociation rate, showing strength of antibody binding, was measured by Langmuir 1:1 binding model fitting from 30 to 600 sec for dissociation. For some of the samples the dissociation rate was not measured due to too low dissociation. In these cases, dissociation rates were extrapolated to 2.0×10⁻⁵, detectable dissociation from 0 to 1200 sec for 5% signal decrease. Avidity index was calculated by the following equation: Avidity index=response/k_{off}. The assay was measured twice for each sample. Average values are shown.

Microneutralization Titer (MNT) Assay

Titers of antibodies neutralizing dengue live virus serotypes 1, 2, 3 and 4 were measured by the method reported by Osorio et al., Lancet Infect Dis 14 (2014), 830-838.

Statistics Analysis

All the data were analyzed with one-way ANOVA by Graph Pad Prizm (ver 7.02 GraphPad software). MNT titer and Avidity index values were converted to Log 10 value and then analyzed by Kruskal-Wallis one-way and ordinary one-way ANOVA, respectively. Response and k_{off} were analyzed by ordinary one-way ANOVA.

Results

Antibody Purification

Forty-eight IgG samples were purified from DEN203 human sera and showed more than 88% purity for each sample. The antibody solution was stored at 4° C. until the assay. The yield of IgG from sera sample were from 4.8 mg/mL to 19.0 mg/mL.

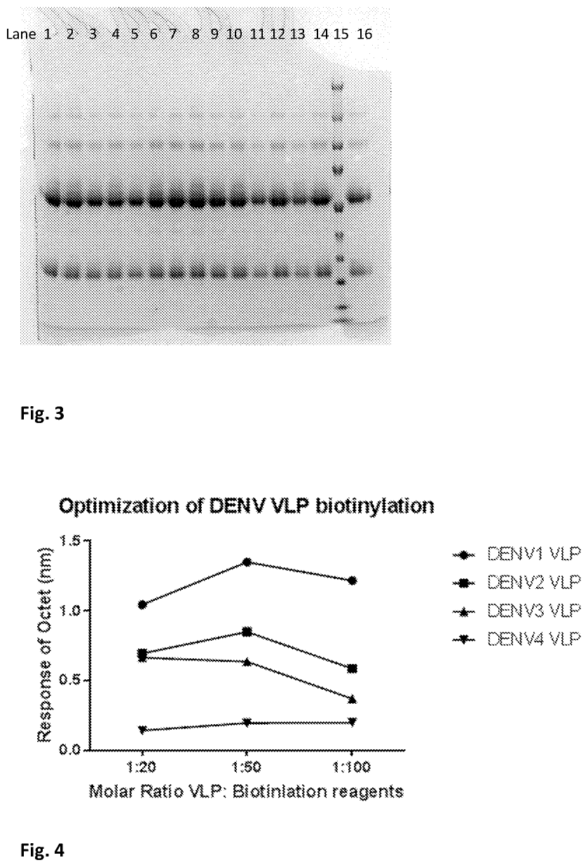

FIG. 3 shows the SDS PAGE analysis of anti-DENV Ab purified from DEN203 sera sample. The distribution of the samples in the lanes of the gel is shown in Table 3.

TABLE 3
Optimization of biotinylation of Dengue VLPs

| Lane | subject ID | days | purity % |
|---|---|---|---|
| 1 | 1053009 | 0 | 91.4 |
| 2 | 1053012 | 28 | 90.5 |
| 3 | 1053009 | 28 | 93.8 |
| 4 | 1053012 | 90 | 91.7 |
| 5 | 1053009 | 90 | 93.4 |
| 6 | 1053012 | 120 | 91.7 |
| 7 | 1053009 | 120 | 91.3 |
| 8 | 1053012 | 180 | 91.6 |
| 9 | 1053009 | 180 | 92.2 |
| 10 | 1053012 | 360 | 91.4 |
| 11 | 1053009 | 360 | 94.6 |
| 12 | 1063001 | 0 | 88.8 |
| 13 | 1053012 | 0 | 92.4 |
| 14 | 1063001 | 28 | 89.7 |
| 16 | 1063001 | 90 | 89.1 |

The Dengue VLP/biotinylated reagents ratio was varied from 1:20, 1:50 to 1: 100 excess molar. Responses, i.e. binding of anti-Dengue antibody, were increased when biotinylated from 20 to 50 excess moles. However, if 100 excess moles were used, the response, binding of anti-Dengue antibodies, was decreased. From these optimizations, the 50 fold excess mole of biotinylation reagents was chosen for 60 min incubations (FIG. 4).

Avidity Assay

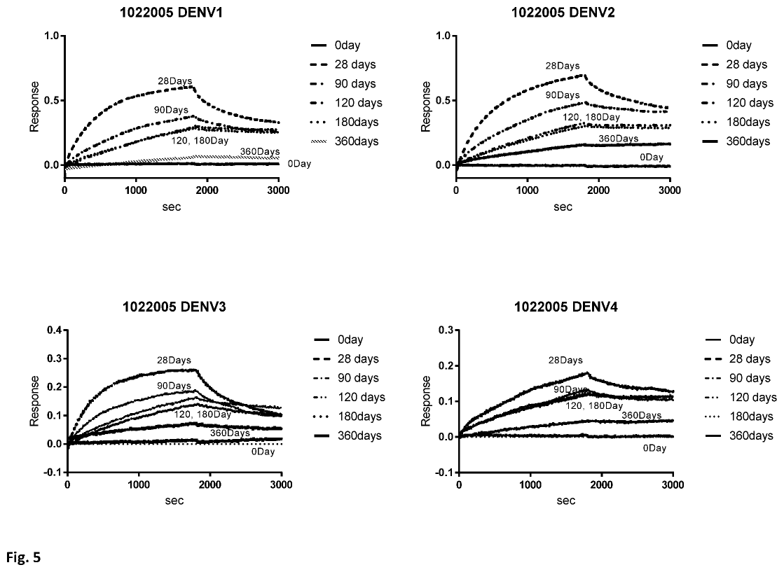

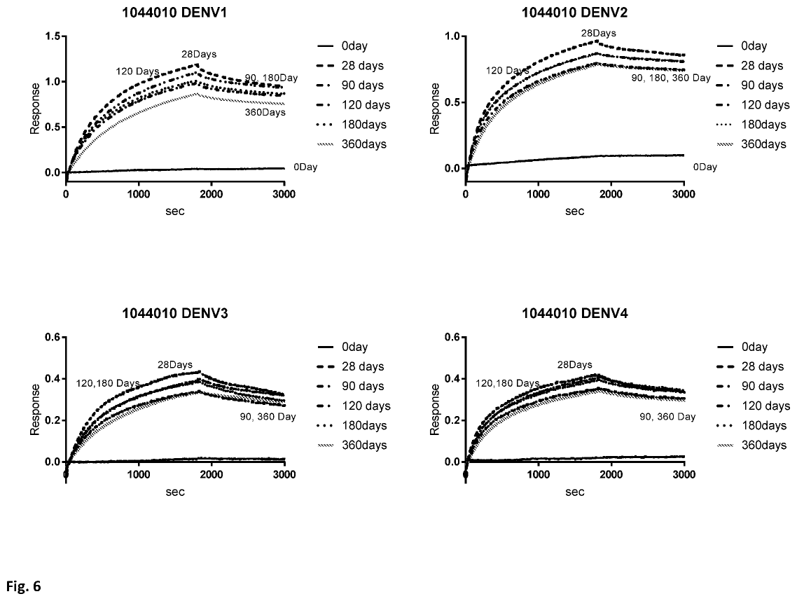

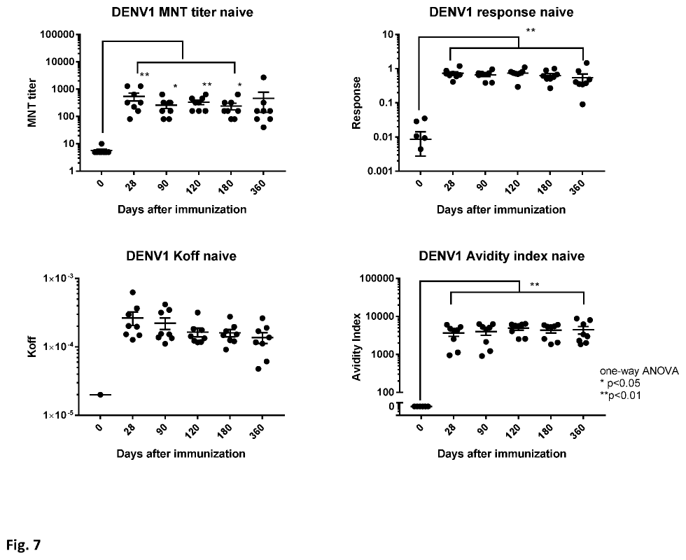

Avidity assays were conducted for samples from eight subjects which were dengue-naïve before vaccination from day 0, 28, 90, 120, 180 and 360 days post vaccination. Hence, 48 samples for each of the four serotypes of Dengue VLP were measured. Two biosensor image patterns that represent featured patterns were shown in FIGS. 5 and 6, respectively.

Subject ID 1022005 showed a strong affinity maturation process. The response to all dengue serotypes was increased at day 28 after immunization, but binding antibodies dissociated easily (Day 28 to DENV2; Response: 0.694 nm, k_{off}: 4.06E-04 l/s and Avidity index: 1721 nm*s; FIG. 5). At day 90, the binding of the antibodies was decreased, but their dissociation from dengue VLP was reduced (Day 90 to DENV2; Response: 0.311 nm, k_{off}: 7.93E-05 l/s and Avidity index: 6524 nm*s; FIG. 5). From that point of view, a clear affinity maturation was observed.

On the other hand, subject ID 1044010 showed high response and small dissociation rate even at day 28. (Day 28 to DENV2; Response: 1.003 nm, k_{off}: 7.65E-05 l/s and Avidity index: 13920 nm*s; FIG. 6) and kept its antibody strength until day 360 (Day 360 to DENV2; Response; 0.782 nm, k_{off}; 4.04E-05 l/s Avidity index; 25920 nm*s; FIG. 6). These data suggest that the vaccination with the dengue vaccine was effective even one year after the immunization. Eight subjects were compared from day 0 to day 360 after vaccination. A summary of the avidity data from these eight subjects is shown in Table 4.

TABLE 4
Summary of Avidity Assay of DEN203
DENV1

| | | MNT | | | response | | | Koff | | | Avidity index | | |
|---|---|---|---|---|---|---|---|---|---|---|---|---|---|
| days | N | GEM | | SEM | MEAN | | SEM | MEAN | | SEM | GEM | | SEM |
| 0 | 8 | 5 | ± | 1 | 0.009 | ± | 0.006 | ND | ± | ND | 0 | ± | 0 |
| 28 | 8 | 364 | ± | 172 | 0.728 | ± | 0.082 | 2.65E−04 | ± | 5.93E−05 | 3065 | ± | 677 |
| 90 | 8 | 207 | ± | 65 | 0.657 | ± | 0.067 | 2.22E−04 | ± | 4.23E−05 | 3238 | ± | 790 |
| 120 | 8 | 293 | ± | 62 | 0.739 | ± | 0.078 | 1.64E−04 | ± | 2.38E−05 | 4632 | ± | 571 |
| 180 | 8 | 190 | ± | 66 | 0.628 | ± | 0.082 | 1.60E−04 | ± | 2.01E−05 | 3924 | ± | 647 |
| 360 | 8 | 175 | ± | 317 | 0.549 | ± | 0.151 | 1.37E−04 | ± | 2.48E−05 | 3741 | ± | 997 |

DENV2

| | | MNT | | | response | | | Koff | | | Avidity index | | |
|---|---|---|---|---|---|---|---|---|---|---|---|---|---|
| Days | N | GEM | | SEM | MEAN | | SEM | MEAN | | SEM | GEM | | SEM |
| 0 | 8 | 5 | ± | 0 | 0.012 | ± | 0.008 | ND | ± | ND | 0 | ± | 0 |
| 28 | 8 | 1396 | ± | 2583 | 0.684 | ± | 0.055 | 1.77E−04 | ± | 4.26E−05 | 4648 | ± | 1474 |
| 90 | 8 | 538 | ± | 294 | 0.634 | ± | 0.041 | 1.37E−04 | ± | 2.73E−05 | 5737 | ± | 2159 |
| 120 | 8 | 562 | ± | 291 | 0.661 | ± | 0.056 | 1.02E−04 | ± | 1.56E−05 | 7473 | ± | 1926 |
| 180 | 8 | 453 | ± | 301 | 0.590 | ± | 0.063 | 9.02E−05 | ± | 1.45E−05 | 7207 | ± | 2054 |
| 360 | 8 | 353 | ± | 133 | 0.531 | ± | 0.115 | 6.75E−05 | ± | 1.32E−05 | 9306 | ± | 2992 |

DENV3

| | | MNT | | | response | | | Koff | | | Avidity index | | |
|---|---|---|---|---|---|---|---|---|---|---|---|---|---|
| Days | N | GEM | | SEM | MEAN | | SEM | MEAN | | SEM | GEM | | SEM |
| 0 | 8 | 5 | ± | 0 | −0.001 | ± | 0.005 | ND | ± | ND | 0 | ± | 0 |
| 28 | 8 | 174 | ± | 144 | 0.291 | ± | 0.030 | 3.65E−04 | ± | 9.87E−05 | 986 | ± | 225 |
| 90 | 8 | 160 | ± | 151 | 0.281 | ± | 0.029 | 2.76E−04 | ± | 6.93E−05 | 1246 | ± | 386 |
| 120 | 8 | 247 | ± | 60 | 0.312 | ± | 0.029 | 2.10E−04 | ± | 1.83E−05 | 1540 | ± | 284 |
| 180 | 8 | 199 | ± | 300 | 0.267 | ± | 0.033 | 2.14E−04 | ± | 3.26E−05 | 1584 | ± | 600 |
| 360 | 8 | 200 | ± | 645 | 0.225 | ± | 0.050 | 1.98E−04 | ± | 2.33E−05 | 1239 | ± | 494 |

DENV4

| | | MNT | | | response | | | Koff | | | Avidity index | | |
|---|---|---|---|---|---|---|---|---|---|---|---|---|---|
| days | N | GEM | | SEM | MEAN | | SEM | MEAN | | SEM | GEM | | SEM |
| 0 | 8 | 5 | ± | 0 | 0.004 | ± | 0.004 | ND | ± | ND | 0 | ± | 0 |
| 28 | 8 | 62 | ± | 45 | 0.257 | ± | 0.035 | 2.42E−04 | ± | 4.15E−05 | 1145 | ± | 299 |
| 90 | 8 | 26 | ± | 11 | 0.224 | ± | 0.028 | 2.06E−04 | ± | 1.45E−05 | 1059 | ± | 193 |
| 120 | 8 | 48 | ± | 21 | 0.260 | ± | 0.031 | 1.57E−04 | ± | 1.84E−05 | 1894 | ± | 498 |
| 180 | 8 | 40 | ± | 18 | 0.227 | ± | 0.035 | 1.70E−04 | ± | 1.41E−05 | 1504 | ± | 235 |
| 360 | 8 | 34 | ± | 39 | 0.196 | ± | 0.047 | 1.17E−04 | ± | 2.63E−05 | 2200 | ± | 773 |

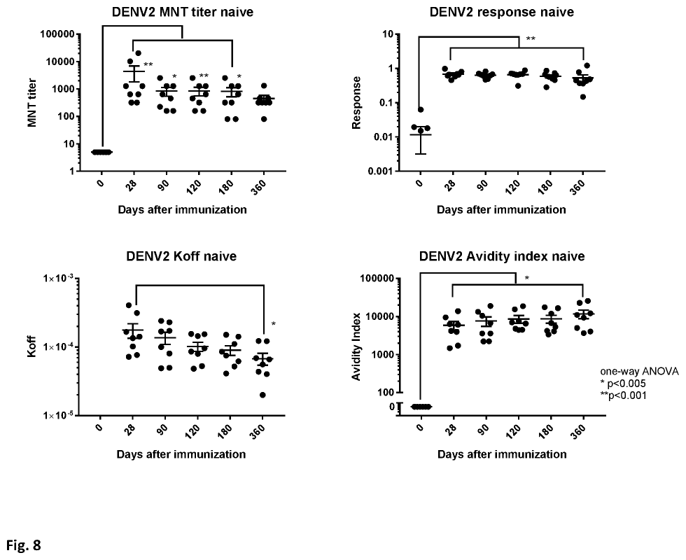

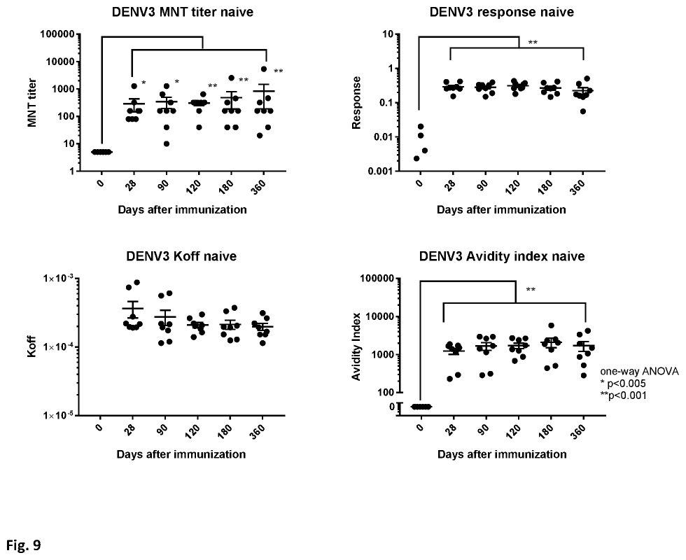

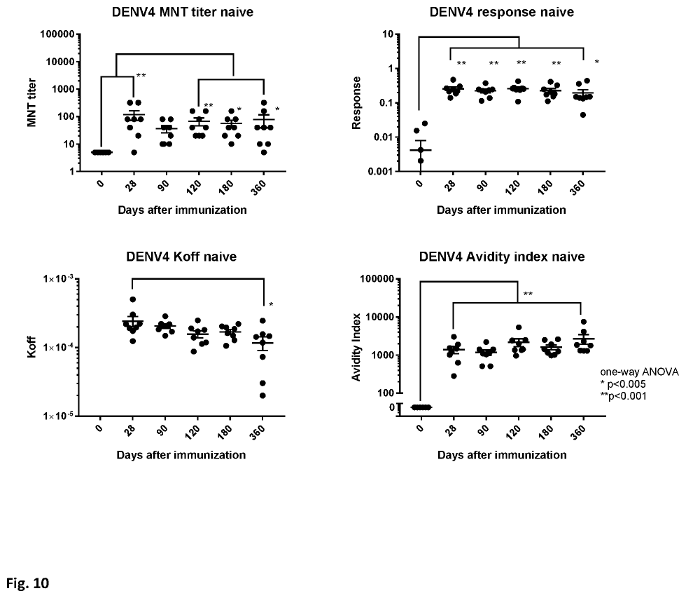

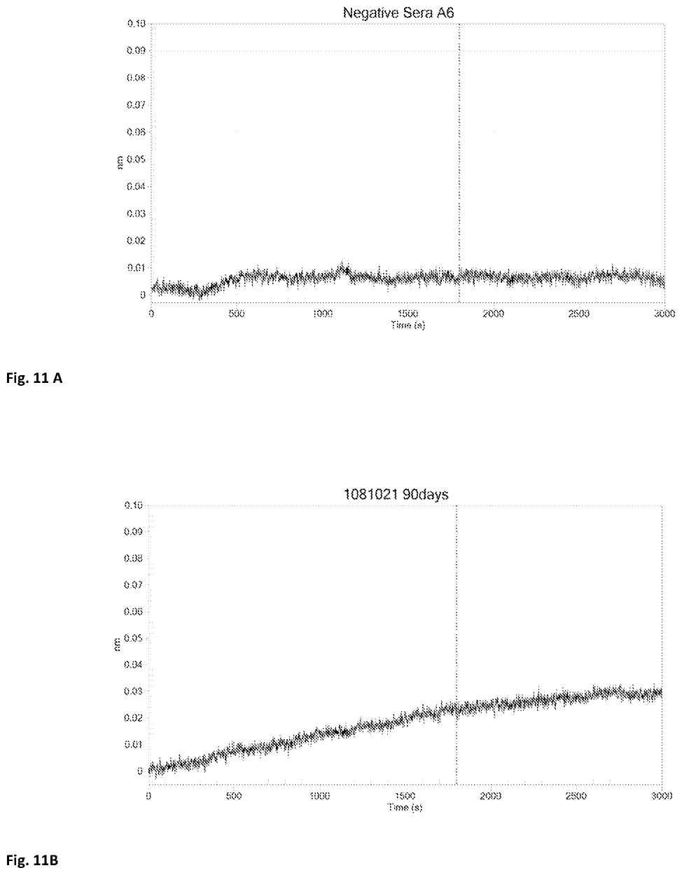

For dengue serotype 2, FIG. 8, MNT titer was increased at day 28 and then gradually decreased. However, the response that reflects anti Dengue 2 IgG content, was increased at day 28, but some of the subjects kept the response even at day 360. There were significant differences between day 0 and day 28 to day 360 (one-way ANOVA p<0.01). Antibody dissociation rate, k_{off}, was decreased from day 28 to day 360, there were significant differences between day 28 and day 360 (by one-way ANOVA p<0.05). These findings showed that the strength of anti-Dengue antibodies kept in patients for over one year was increased over time. The avidity index, showing strength of antibody binding was still at a high level at day 360. There were also significant differences between day 0 and day 28 to day 360 (by one-way ANOVA p<0.05). There were almost the same responses towards all serotypes, DENV1, 3 and 4 respectively, FIGS. 7, 9 and 10. These findings suggest that immunization with the dengue vaccine sustains immune responses and these high antibody avidities may protect from infections by dengue virus.

### Example 2: Reactivity and Specificity of Avidity Assay Using Anti-DENV Antibody Panels (VLP/AR2G, VLP/SA Biosensor)

Material and Methods

Materials

Dengue serotype-1, 2 3 and 4 were purchased from Native antigen and SA biosensor, AR2G biosensor and The Amine Reactive 2nd Generation (AR2G) Reagent Kit were purchased from Forte Bio, EZ-Link Sulfor-NHS_Biotin were purchased from Thermo Scientific. Anti-Dengue antibodies were purchased or prepared based on amino acid sequences or hybridomas. A11, B7, C10, 2C8, 4G2, DV1-106, 2D22, 5J7, DV4-75, DV3-E60, WNV E60 DV1-106, 1M7 shown in Table 5.

TABLE 5
anti DENV monoclonal antibody panels

| | Epitope | |
|---|---|---|
| Clones | Specificity | Reference |
| A11 | EDE | Dejnirattisai, W. Wongwiwat, S. Supasa, X. Zhang, X. |
| B7 | Cross | Dai, A. Rouvinsky, A. Jumnainsong, C. Edwards, N. T. |
| C10 | reactive | Quyen, T. Duangchinda, J. M. Grimes, W. Y. Tsai, C. Y. |
| 2C8 | | Lai, W. K. Wang, P. Malasit, J. Farrar, C. P. Simmons, Z. H. |
| | | Zhou, F. A. Rey, J. Mongkolsapaya, G. R. Screaton |
| | | A new class of highly potent, broadly neutralizing |
| | | antibodies isolated from viremic patients infected with |
| | | dengue virus |
| | | Nat. Immunol., 16 (2015), pp. 170-177 |
| 4G2 | Fusion | Halstead S B, Venkateshan C N, Gentry M K, Larsen L K. |
| | Loop | Heterogeneity of infection enhancement of dengue 2 |
| | Cross | strains by monoclonal antibodies. |
| | reactive | J Immunol. 1984 March; 132(3):1529-32 |
| DV-106 | QE | Shrestha B, Brien J D, Sukupolvi-Petty S, Austin S K, |
| | DENV1 | Edeling M A, Kim T, O'Brien K M, Nelson C A, Johnson S, |
| | Specific | Fremont D H, Diamond M S. |
| | | The development of therapeutic antibodies that neutralize |
| | | homologous and heterologous genotypes of dengue virus |
| | | type 1. |
| | | PLOS Pathog. 2010 Apr. 1; 6(4): e1000823 |
| 2D22 | QE | R. de Alwis, S. A. Smith, N. P. Olivarez, W. B. Messer, J. P. |
| 5J7 | DENV2 | Huynh, W. M. Wahala, L. J. White, M. S. Diamond, R. S. |
| | specific | Baric, J. E. Crowe Jr., A. M. de Silva |
| | | Identification of human neutralizing antibodies that bind to |
| | | complex epitopes on dengue Virions |
| | | Proc. Natl. Acad. Sci. U.S.A., 109 (2012), pp. 7439-7444 |
| WNV-E60 | Fusion | Oliphant T, Engle M, Nybakken G E, Doane C, Johnson S, |
| | loop Cross | Huang L, Gorlatov S, Mehlhop E, Marri A, Chung K M, |
| | reactive | Ebel G D, Kramer L D, Fremont D H, Diamond M S. |
| | | Development of a humanized monoclonal antibody with |
| | | therapeutic potential against West Nile virus. |
| | | Nat Med. 2005 May; 11(5): 522-30 |
| DV3-E60 | QE | Brien J D, Austin S K, Sukupolvi-Petty S, O'Brien K M, |
| | DENV3 | Johnson S, Fremont D H, Diamond M S, |
| | specific | Genotype-specific neutralization and protection by |
| | | antibodies against dengue virus type 3 |
| | | J Virol. 2010 Oct; 84(20): 10630-43 |
| DV4-75 | QE | Sukupolvi-Petty S, Brien J D, Austin S K, Shrestha B, |
| | DENV4 | Swayne S, Kahle K, Doranz B J, Johnson S, Pierson T C, |
| | specific | Fremont D H, Diamond M S. |
| | | Functional analysis of antibodies against dengue virus type |
| | | 4 reveals strain-dependent epitope exposure that impacts |
| | | neutralization and protection. |
| | | J Virol. 2013 August; 87(16): 8826-42 |
| 1M7 | Fusion | Smith S A, de Alwis A R, Kose N, Harris E, Ibarra K D, |
| | loop Cross | Kahle K M, Pfaff J M, Xiang X, Doranz B J, de Silva A M, |
| | reactive | Austin S K, Sukupolvi-Petty S, Diamond M S, Crowe J E Jr. |
| | | The potent and broadly neutralizing human dengue virus- |
| | | specific monoclonal antibody 1C19 reveals a unique cross- |
| | | reactive epitope on the bc loop of domain II of the |
| | | envelope protein. |
| | | MBio. 2013 Nov. 19; 4(6): e00873-13 |

Evaluation of Dengue VLP Coupled to AR2G Biosensor Using Anti-Dengue Antibody Panel

This assay was measured by Octet Red (Forte Bio). Coupling Dengue VLP to AR2G biosensor following the instructions of the Amine Reactive 2^{nd} Generation (AR2G) reagent kit. First, AR2G biosensor was hydrated with AR2G in PBS for 5 min before the reaction. AR2G biosensor was activated with 20 mM EDC (1-Ethyl-3-[3-dimethylaminopropyl] carbodiimide hydrochloride) and 10 mM S—NHS (N-hydroxysulfosuccinimide) for 300 sec. The activated biosensor was reacted with 10 ug/mL Dengue VLP-1, -2, -3 and -4 in 10 mM Acetate pH 6 buffer for 600 sec respectively. The VLP coupled biosensor was quenched with 1M ethanolamine pH 8.5 for 300 sec. All reactions were done at 1000 rpm plate shaking at 30° C. After the coupling of Dengue VLP to the biosensor, binding to the anti-Dengue antibody panels was confirmed. The antibody solution was diluted to 10 ug/mL in 0.1% BSA PBST. These antibodies were associated to Dengue VLP for 900 sec and these antibodies were dissociated in the same buffer for 1800 sec. This assay was conducted at 30° C. with 1000 rpm shaking plate. All solution volume was 200 uL in 96 well black plate (Greiner Bio).

Biotinylation of Dengue VLP

200 ug of Dengue Virus Like Particle (VLP), type 1, 2, 3 and 4 (Native Antigen) was biotinylated with 50 excess moles of EZ-Link Sulfo-NHS-Biotin (Thermo) following the manufacturer's protocol. The reaction took place at room temperature for 60 min, after the reaction, the excess biotinylation reagents were removed and these biotinylated VLPs buffer-exchanged with D-PBS using Amicon Ultra 4 (Millipore MWCO 30 KDa).

Evaluation of Biotinylated Dengue VLP/SA Biosensor Using Anti-Dengue Antibody Panel

This assay was measured by Octet Red (Forte Bio). SA biosensor was hydrated with PBS for 5 min before the assay. Biotinylated Dengue VLP was diluted to 5 ug/mL in 0.1% BSA PBST and bound to SA Biosensor for 600 sec. then binding to the anti-Dengue antibody panels was confirmed. The antibody solution was diluted to 10 ug/mL in 0.1% BSA PBST. These antibodies were associated to Dengue VLP for 600 sec and these antibodies were dissociated in the same buffer for 900 sec. This assay was conducted at 30° C. with 1000 rpm shaking plate. All solution volume was 200 uL in 96 well black plate (Greiner Bio).

Results

The results are shown in the following Table 6.

TABLE 6
Reactivity of anti-Dengue
antibodies panels to AR2G Dengue VLPs

| Clones | Specificity | Epitope | DENV1 | DENV2 | DENV3 | DENV4 |
|---|---|---|---|---|---|---|
| 4G2 | CR | Fusion | +++ | ++ | +++ | +++ |
| | | Loop | | | | |
| WNV- | CR | Fusion | ++ | ++ | +++ | ++ |
| E60 | | Loop | | | | |
| 2D22 | DENV2 | QE | − | ++ | − | − |
| DV3 | DENV3 | QE | − | − | ++ | − |
| E60 | | | | | | |
| DV4 | DENV4 | QE | − | − | + | +++ |
| 75 | | | | | | |

−; no binding
+; weak binding
++; binding
+++; Strong binding
EDE; Envelope Dimer Epitope,
QE; Quaternary E-protein,
CR; Cross reactive,
VLP; Virus like particle

The cross-reactive antibodies 4G2 and WNV-E60 bound to each of DENV1 to DENV4 attached to the AR2G biosensor. The serotype-specific antibodies 2D22, DV3E60 and DV475 only bound to its serotype-specific DENV attached to the AR2G biosensor but did not bind to VLPs from other Dengue serotypes.

TABLE 7
Reactivity of anti-Dengue antibodies panels to SA
biosensor to biotinylated Dengue VLPs

| clones | epitope | specificity | DENV1 | DENV2 | DENV3 | DENV4 |
|---|---|---|---|---|---|---|
| 4G2 | Fusion | CR | +++ | +++ | ++ | ++ |
| | Loop | | | | | |
| WNV- | Fusion | CR | ++ | +++ | ++ | + |
| E60 | Loop | | | | | |
| 1M7 | Fusion | CR | +++ | +++ | +++ | ++ |
| | Loop | | | | | |
| DV1-106 | QE | DEN1 | + | − | − | − |
| 2D22 | QE | DEN2 | − | + | − | − |
| 5J7 | QE | DEN3 | − | − | +++ | − |
| DV4-75 | QE | DEN4 | − | − | − | +++ |

−; no binding
+; weak binding
++; binding
+++; Strong binding
EDE; Envelope Dimer Epitope,
QE; Quaternary E-protein,
CR; Cross reactive,
VLP; Virus like particle

These results demonstrate the suitability of AR2G coupling VLP strategies and biotinylated VLP strategies for avidity assays.

### Example 3: Propagation of Live Dengue 3 and 4 Virus

Materials and Methods

Materials

Dengue Wildtype strain type 16562 for Dengue 3 and wildtype strain type 1036 for Dengue 4 were used. Vero cells were obtained from WHO. Coring cell stacker 10 layer were purchased form Coring. For cell culture medium, Fetal bovine Serum, FBS, was obtained from Sigma Aldrich and DMEM 1X and Penicillin/Streptomycin solution were purchased from Gibco. For concentration, Viva Flow MWCO 100 kDa system were purchased from Sartorius. 60% sucrose solution and TNE buffer, Tris 10 mM, EDTA 1 mM, NaCl 100 mM pH8.0 were obtained from Teknova.

Preparation of Vero Cells

Vero cells were cultured in 10-layer hyperflask cell culture vessel with 10% FBS, DMEM and Pen/Strep Medium and confluent to vessels prior to the transfection.

Infection and Purification of Dengue Virus

Wildtype DENV strains, type 16562 for Dengue 3 and type 1036 for Dengue 4, was propagated in a 10-layer hyperflask cell culture vessel at a MOI of 0.01 using Vero cells for an incubation period of 9 days. Supernatants were collected at days 5, 7 and 9 post-infection and cells replenished with fresh virus growth media after each collection time point. Each collection day supernatant was clarified and filtered using Millipore 0.22 um filters to remove host cell debris. The clarified and filtered supernatants were subjected to tangential flow filtration, TFF Viva Flow MWCO 100 kDa, to concentrate virus stock and concentrated solution were overlaid with 20% Sucrose and centrifuged for 3 hours at 112,398×g (25,000 RPM), 4° C. to form the pellets. The formed virus pellets were resuspended in TNE buffer and frozen down at −80° C. The concentration of the viruses was estimated using BCA protein assay kit (Themo) for BSA as a standard.

SDS-PAGE

The purity of Dengue virus was confirmed by SDS PAGE (NuPAGE 4-12% Bis-Tris Gel, Thermo) 2 ug protein sample were reduced at 70° C. for 10 min and applied to the gel. These gels were stained by simple stain blue (Thermo) and these images were captured by ChemoDoc Touch imaging system (Bio-Rad).

Results

Live dengue virus was obtained from the propagation process with Vero cells. These purified viruses showed E protein (MW 55 kDa) and prM protein (MW 18 KDa) in SDS-PAGE.

### Example 4: Reactivity and Specificity of Avidity Assay Using Anti-DENV Antibody Panels

Materials

Dengue VLPs for serotype-3 and 4 were purchased from Native antigen and Live Dengue virus were propagated and purified as described in Example 5. SA biosensor, APS biosensor and Protein G biosensor were purchased from Forte Bio, EZ-Link Sulfor-NHS_Biotin were purchased from Thermo Scientific. Anti-Dengue antibodies were purchased or prepared based on amino acid sequences or hybridomas. All, B7, C10, 2C8, 4G2, 2D22, DV4-75, DV3-E60, WNV E60 DV1-106, 5J7 and 1M7 are described in Table 5.

Biotinylation of Dengue VLP

200 ug of Dengue Virus Like Particle, VLP, type 3 and 4 (Native Antigen) were biotinylated with 50 excess moles of EZ-Link Sulfo-NHS-Biotin (Thermo) following the manufacturer's protocol. The reaction took place at room temperature for 60 min, after the reaction, the excess biotinylation reagents were removed and these biotinylated VLPs buffer-exchanged with D-PBS using Amicon Ultra 4 (Millipore MWCO 30 kDa).

Biotinylation of Anti-Dengue Monoclonal Antibody 4G2

50 ug of anti-Dengue antibody, 4G2, cross-reactive antibody to all serotypes, was biotinylated with 50 excess moles of EZ-Link Sulfo-NHS-Biotin (Thermo) following the manufacturer's protocol. The reaction was carried out at room temperature for 60 min, after the reaction, the excess biotinylation reagents were removed and these biotinylated VLPs buffer-exchanged with D-PBS using Amicon Ultra 4 (Millipore MWCO 30 kDa).

Evaluation of Live Virus and VLP Binding to Anti-Dengue Antibody/Protein G Biosensor

This assay was measured by Octet Red (Forte Bio). Protein G biosensor was hydrated with PBS for 5 min before the assay. The biosensor was dipped into 1 ug/mL of twelve anti-Dengue antibodies in 0.1% PBS and PBST for 600 sec. Then binding to Dengue live virus or VLP was confirmed. Live virus and VLP were diluted to 5 ug/mL in 0.1% BSA PBST. These solutions were associated to anti-Dengue antibody panel/Protein A biosensor for 900 sec and dissociated in the same buffer for 1800 sec. This assay was conducted at 30° C. with 1000 rpm shaking plate. All solution volume was 200 uL in 96 well black plate (Greiner Bio).

Evaluation of Anti-Dengue Antibody Panels Binding to Live Virus to APS Biosensor

This assay was measured by Octet Red (Forte Bio). APS biosensor was hydrated with PBS for 5 min before the assay. Dengue VLP or Live virus were diluted to 3 ug/mL in PBS and bound to APS Biosensor for 600 sec. Then the sensor was blocked with 1% BSA PBS for 300 sec. The binding to twelve anti-Dengue antibody panels was confirmed. The antibody solution was diluted to 10 ug/mL in 1% BSA PBS. These antibodies were associated to Dengue VLP or Live virus for 900 sec and these antibodies were dissociated in the same buffer for 1800 sec. This assay was conducted at 30° C. with 1000 rpm shaking plate. All solution volume was 200 uL in 96 well black plate (Greiner Bio).

Evaluation of Anti-Dengue Antibody Panels Binding to Biotinylated VLP to SA Biosensor.

This assay was measured by Octet Red (Forte Bio). SA biosensor was hydrated with PBS for 5 min before the assay. Biotinylated Dengue VLP was diluted to 5 ug/mL in 0.1% BSA PBST and bound to SA Biosensor for 600 sec. Then binding to twelve anti-Dengue antibody panels was confirmed. The antibody solution was diluted to 10 ug/mL in 0.1% BSA PBST. These antibodies were associated to Dengue VLP for 900 sec and these antibodies were dissociated in the same buffer for 1800 sec. This assay was conducted at 30° C. with 1000 rpm shaking plate. All solution volume was 200 uL in 96 well black plate (Greiner Bio).

Results

We confirmed Dengue live virus and VLP binding to anti-Dengue antibody panel.

TABLE 8
Reactivities of anti-Dengue antibody panels
to various avidity assay format of DENV3

| | | | APS LV/VLP | | |
|---|---|---|---|---|---|
| | | | capture | | Biotinylated |
| | | | Live | | VLP |
| Clone | Epitope | specificity | Virus | VLP | VLP |
| 1M7 | Fusion | CR | + | +++ | +++ |
| | Loop | | | | |
| WNV- | Fusion | CR | + | ++ | ++ |
| E60 | Loop | | | | |
| DV1- | QE | DEN1 | − | − | − |
| 106 | | | | | |
| 2D22 | QE | DEN2 | + | − | − |
| 5J7 | QE | DEN3 | − | +++ | +++ |
| DV3- | QE | DEN3 | ++ | + | − |
| E60 | | | | | |
| DV4-75 | QE | DEN4 | − | − | − |

−; no binding +; weak binding ++; binding +++; Strong binding
EDE; Envelope Dimer Epitope, QE; Quaternary E-protein, CR; Cross reactive, VLP; Virus like particle

TABLE 9
Reactivities of anti-Dengue antibody panels
to various avidity assay format of DENV4

| | | | APS LV/VLP | | |
|---|---|---|---|---|---|
| | | | capture | | Biotinylated |
| | | | Live | | VLP |
| clone | epitope | specificity | Virus | VLP | VLP |
| 1M7 | Fusion | CR | + | ++ | ++ |
| | Loop | | | | |
| DV1- | QE | DEN1 | − | − | − |
| 106 | | | | | |
| 2D22 | QE | DEN2 | − | − | − |
| 5J7 | QE | DEN3 | − | − | − |
| DV3- | QE | DEN3 | − | − | − |
| E60 | | | | | |
| DV4-75 | QE | DEN4 | + | ++ | +++ |

−; no binding +; weak binding ++; binding +++; Strong binding
EDE; Envelope Dimer Epitope, QE; Quaternary E-protein, CR; Cross reactive, VLP; Virus like particle

When IgG was captured to Protein G biosensor, these cross-reactive antibody panels and Dengue 3 or 4 specific antibodies reacted with both live virus.

### Example 5: Anti-Dengue Avidity Assay Using Live Virus

Materials and Methods

Materials

Live Dengue virus serotype 3 were propagated and purified as outlined above. APS biosensor was purchased from Forte Bio. Dengue positive control sera and negative control sera were obtained from NIH. Sera samples were selected from DEN-203, phase 2 clinical trial from Puerto Rico, Colombia, Singapore, and Thailand. The sera were stored at −80° C. until use. These sera samples were thawed at 4° C. storage shelf overnight before purification.

Antibody Purification

IgG were purified from these 200 uL sera by Protein G Sepharose (GE). Briefly, 200 uL of sera were mixed with 3 mL D-PBS and 0.6 mL 50% Protein G Sepharose in 15 mL centrifuge tube. These centrifuged tubes were mixed for 90 min at room temperature with shaker. After centrifuging, Protein G Sepharose slurry were transferred to 24 well Unifilter (GE) and the gel was washed with D-PBS for 4 times and eluted with 0.1M Glycine HCl pH2.7 for 4 times. The eluates were immediately neutralized to pH 7.0 to 7.5 with 1M Tris HCl pH8.0. the solution was buffer-exchanged to Amicon Ultra 4 (Millipore MWCO 30 KDa). These antibody solutions are measured at A280 by NanoDrop 2000 (Thermo) and the IgG concentration was calculated. Then these samples were diluted with D-PBS to 2.5 mg/mL for each sample ID. The antibody purity was confirmed by SDS PAGE (NuPAGE 4-12% Bis-Tris Gel, Thermo). 2 ug protein sample were reduced at 70° C. for 10 min and applied to the gel. These gels were stained by simple stain blue (Thermo) and these images were captured by ChemoDoc Touch imaging system (Bio-Rad) and calculated purity was confirmed more the 80% for both IgL and IgH band by Image Lab software (Bio-Rad).

Anti-Dengue Avidity Assay Using Live Virus/APS Biosensor

Avidity assays were measured by Octet 96 red using APS biosensor (Forte Bio). APS biosensors were hydrated with D-PBS at least 10 min before an analysis. 5 ug/mL Dengue Live virus serotype 3 in PBS was captured with APS biosensor for 600 sec and 1% BSA PBS were blocked on the surface for 600 sec. 250 ug/mL anti-DENV polyclonal antibodies purified from DEN203 patients' sera in 1% BSA PBS were bound to the biosensor to 1800 sec and then the sensors were incubated in 1% BSA PBS for 1200 sec to dissociate the binding antibody. These reactions were conducted at 30° C. and assay plate, 200 uL/well, were shaken for 1000 rpm. For the negative subtractions, a double subtraction protocol was applied with a combination of antibody/DENV Live virus, antibody/no DENV Live virus, no antibody/DENV Live virus and no antibody/no DENV Live virus to assess the dissociation rate precisely.

Results

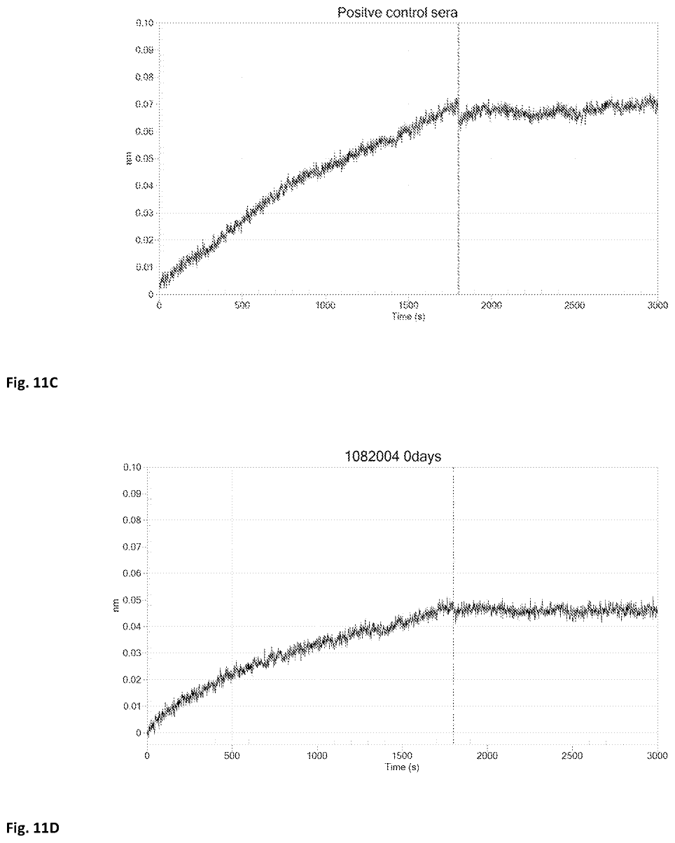

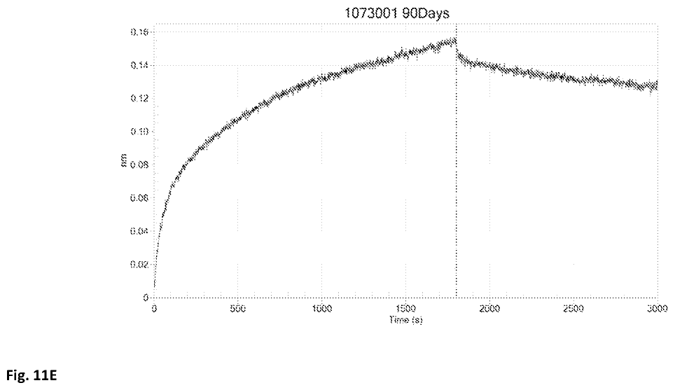

We conducted the avidity assay using Live virus serotype 3 and negative and positive control sera and DEN203 clinical trial samples. All sera were purified IgG by Protein G sepharose to reduce non-specific bindings. There were no signals from IgG negative sera (FIG. 11A), however, for positive sera (FIG. 11C) and DEN203 samples were observed binding antibody to live virus and antibody dissociation. (FIG. 11 B,D,E)

The results described in Examples 6 to 9 below were obtained with sera from subjects treated in the DEN-203 clinical trial (ClinicalTrials.gov identifier NCT01511250), i.e. a phase 2 clinical trial in Puerto Rico, Colombia, Singapore, and Thailand and sera from days 0, 120 and 180 days post vaccination were obtained from these subjects. The subjects were treated with a tetravalent dengue composition comprising live attenuated dengue-1, dengue-2, dengue-3 and dengue-4 viruses. The live attenuated dengue-1, dengue-3 and dengue-4 viruses are chimeric viruses with a dengue-2 backbone, wherein the prM/E part of dengue-2 is replaced by the prM/E part of dengue-1, dengue-3 or dengue-4, respectively. The subjects received two doses of the tetravalent dengue composition comprising live attenuated dengue-1, dengue-2, dengue-3 and dengue-4 viruses on day 0 and day 90.

### Example 6: Microneutralization Test (Neutralization Assay)

Immunogenicity was measured by a plaque reduction neutralization test (PRNT) as described in the WHO Guidelines (World Health Organization Department of Immunization Vaccines Biologicals (2007) Guidelines for plaque reduction neutralization testing of human antibodies to dengue viruses, WHO/IVB/07.07) to each one of the four dengue serotypes with titers defined as the dilution resulting in a 50% reduction in plaque values (PRNT50). Within this application the values PRNT50 and MNT50 are used interchangeably.

Briefly, serial dilutions of the heat-inactivated antibody-containing test and control sera samples (dilutions range 1:10 to 1:20480) were prepared and mixed with a constant concentration of dengue viruses, in particular DENV-1 strain 16007, DENV-2 strain 16681, DENV-3 strain 16562 and DENV-4 strain 1036, and incubated for two hours at room temperature to enable the neutralization of the virus by the antibodies present in the sera. After the incubation the mixture of virus and antibodies was transferred onto 96 well plates with confluent Vero cells (seeded 48 hours before at a density of 2×10⁵ cells/mi) and the plates were incubated at 37° C. for 1 hour to infect the Vero cells. A 0.75% methyl cellulose overlay was applied to the plate to restrict spread of progeny virus and the plate was incubated for five days at 37° C. to allow virus plaques to develop. After incubation the overlay and culture medium were removed and the cells were fixed by filling the wells with 1 cold 1:1 methanol-ethanol solution. Plates were incubated at −20° C. for 30 minutes and then washed three times with PBS. Afterwards, the plaques were stained with an anti-dengue antibody and a secondary enzyme-labelled antibody binding to the anti-dengue antibody. The MNT50 value was determined using linear regression.

### Example 7: Anti-Dengue NS1 IgG ELISA

Serum samples from subjects treated in the DEN-203 clinical study with a tetravalent composition comprising live attenuated dengue viruses of serotypes 1, 2, 3 and 4 were investigated as described below.

The microplates were coated with 100 μl/well of DENV NS1 protein diluted in 0.1M bicarbonate/carbonate buffer to a final concentration of 1 ng/μl for at least 16 hours and up to one week at a temperature of 2-8° C. NS1 proteins from different serotypes were used individually to coat the microplates. Each plate was washed with PBST (PBS with 0.1% Tween 20) three times and then blocked with blocking buffer (SuperBlock T20, available from ThermoFisher) for 60 minutes at 37° C. Afterwards the plates were washed three times with PBST and serum samples diluted with PBST were added and the plates were incubated for 60 minutes at 37° C. After binding of the antibodies from the serum samples the plates were washed three times with PBST and 100 μl of a goat-anti-human IgG antibody labelled with horseradish peroxidase were added to each well. The plates were incubated for 60 minutes at 37° C., washed three times with PBST and then 100 μl of 1×ABTS peroxidase substrate (Seracare) were added to each well. The reaction was allowed to develop for 15 to 16 minutes at room temperature protected from light, before 50 μl of an ABTS peroxidase stop solution (Seracare) were added to each well. The microplates were read at OD405 on Molecular Devices using software Softmax Pro 7.0.3 within 20 minutes of adding stop solution.

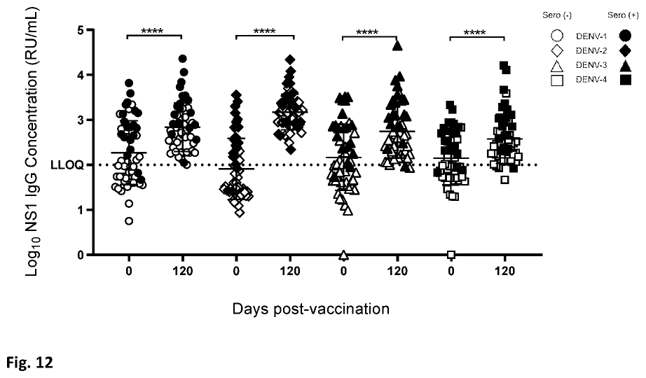

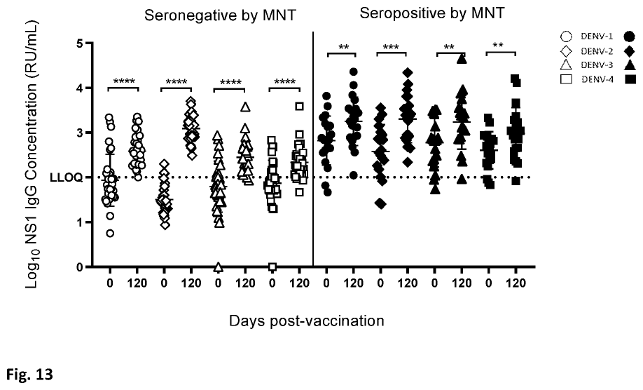

Treatment with a tetravalent composition comprising live attenuated dengue viruses of serotypes 1, 2, 3 and 4 significantly increased IgG responses to DENV-1, 2, 3 and 4 NS1 in the study population 120 days after treatment (FIG. 12). When data were stratified into DENV-seropositive and DENV-seronegative vaccine recipients, IgG responses to all four DENV serotypes increased significantly in both sub-populations, though the increase was greater in seronegative vaccine recipients, because the majority of seropositive vaccine recipients had NS1-specific antibodies prior to vaccination (FIG. 13).

### Example 8: Dengue Total Binding IgG ELISA

Microtiter plates were coated with 100 ng/well of 4G2 antibody (obtained from Absolute Antibody) in 0.1 M carbonate buffer pH 9.6 and incubated overnight at 4° C. After washing three times with PBST, the plates were blocked with 100 μl/well of blocking buffer (SuperBlock T20, available from ThermoFisher) for one hour at 37° C. The diluted dengue virus strains DENV-1 16007, DENV-2 16681, DENV-3 16562 and DENV-4 1036 were added to separate wells of the microtiter plate and the plates were incubated for 90±15 minutes at 37±2° C. After incubation the plates were washed three times with PBST and diluted serum samples were added to each well. The plates were incubated for 60±15 minutes at 37±2° C. and then washed three times with PBST. Goat anti-human Fc IgG conjugated with horseradish peroxidase was added to the plates which were then incubated for 60±15 minutes at 37±2° C. After the plate was washed three times with PBST, 100 μl ABTS peroxidase substrate was added to each well. The reaction was allowed to develop for 15 to 16 minutes at room temperature protected from light, before 50 μl of an ABTS peroxidase stop solution (Seracare) were added to each well. The microplates were read at OD405 on Molecular Devices using software Softmax Pro 7.0.3 within 20 minutes of adding stop solution.

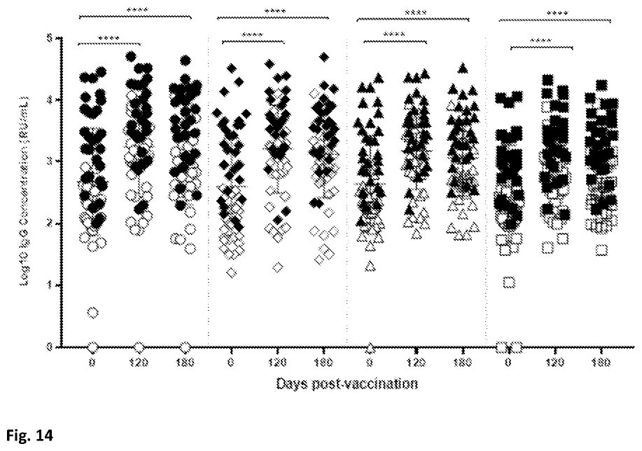

Vaccination significantly increased IgG responses to DENV-1, 2, 3 and 4 virions in the DEN-203 study population (FIG. 14), which remained elevated through day 180 post-vaccination.

### Example 9: Correlation Between the Titers of Neutralizing Antibodies and the Avidity Index

1) Materials and Methods

a) Samples and Reagents

Serum samples of 24 base line seronegative and 19 base line seronegative TAK-003 vaccinated volunteer of DEN-203 clinical trial (ClinicalTrials.gov identifier NCT01511250), i.e. a phase 2 clinical trial from Puerto Rico, Colombia, Singapore, and Thailand were used. Sample selection was based on availability of sera from Day 0, 28, 90, 120, 180, and Day 360 time points. The sera were stored at −80° C. until use and thawed at 4° C. storage overnight before purification. Dengue Virus Like particles were purchased from Native antigen; Dengue 1 strain: Nauru/Western Pacific/1974 (UniProtKB/Swiss-Prot: P17763.2), Dengue 2 strain: Thailand/16681/84 (UniProtKB/Swiss-Prot: P29990.1), Dengue 3 strain: Sri Lanka/1266/2000 (UniProtKB/Swiss-Prot: Q6YMS4.1) and Dengue 4 strain: Dominica/814669/1981 (UniProtKB/Swiss-Prot: P09866.2). 20% of Envelope protein, E-protein, were replaced by the corresponding sequence of Japanese encephalitis strain SA-14 (UniProtKB/Swiss-Prot: P27395.1), amino acid sequence 397-495. SA-Biosensor, biosensor coated with Streptavidin, was purchased from Forte Bio.

b) Antibody Purification

IgG were purified from 200 μL of sera by Protein G Sepharose (GE). Briefly, 200 μL of sera were mixed with 3 mL Dulbecco's Phosphate-buffered saline (D-PBS) and 0.6 mL 50% Protein G Sepharose in a 15 mL centrifuge tube. These centrifuged tubes were mixed for 90 min at room temperature with a shaker. After centrifugation, Protein G Sepharose slurry was transferred to 24 well Unifilter (GE). The slurry was washed with D-PBS 4 times and eluted with 0.1M Glycine HCl pH2.7 for 4 times. The eluates were immediately neutralized to pH 7.0 to 7.5 with 1M Tris HCl pH8.0. The solution was buffer-exchanged with Amicon Ultra 4 (Millipore MWCO 30 KDa). The absorption at 280 nm in these antibody solutions was measured by NanoDrop 2000 (Thermo) and the IgG concentration was calculated. Then these samples were diluted with D-PBS to 2.5 mg/mL for each sample ID. The antibody purity was confirmed by SDS PAGE (NuPAGE 4-12% Bis-Tris Gel, Thermo). 21 μg protein sample was reduced at 70° C. for 10 min and applied to the gel. These gels were stained by simple stain blue (Thermo) and these images were captured by ChemoDoc Touch imaging system (Bio Rad) and calculated purity was confirmed more than 80% from both IgL and IgH band by Image Lab software (Bio-Rad).

c) Preparation of Biotinylated Dengue VLP

For DEN203 avidity assays, 200 μg of Dengue Virus Like Particle, VLP, type 1, 2, 3 and 4 (Native Antigen) were biotinylated with 50 excess moles of EZ-Link Sulfo-NHS-Biotin (Thermo) following the manufacturer's protocol. The reaction took place at room temperature for 60 min, after the reaction, the excess biotinylation reagents were removed and these biotinylated VLPs buffer-exchanged with D-PBS using Amicon Ultra 4 (Millipore MWCO 30 kDa). The biotinylated Dengue VLP solutions were adjusted to a concentration of 0.4 mg/mL and were stored at −80° C. until use.

d) Avidity Assay

Antibody avidity was measured using the Octet HTX systems (FortéBio), and the SA Biosensor. Details are described in Supplementary materials and methods. Briefly, biotinylated dengue VLPs (5 μg/mL) in 0.1% BSA-PBST were captured with the SA Biosensor for 600 sec, then 50 μg/mL Biocytin (ThermoFisher Scientific) was blocked with excess SA for 200 sec. Anti-dengue polyclonal antibodies (125 μg/mL) purified from the serum of DEN-203 volunteers in 0.1% BSA-PBST was bound to the SA Biosensor for 1800 seconds, the sensors were then incubated in 0.1% BSA-PBST 0.35 M NaCl for 1200 seconds to dissociate bound antibody. This assay was conducted at 30° C. with agitation (plate shaker, 1000 rpm). Data were analyzed by double subtraction and using Octet Data Analysis Software HT (version 11.1.2.48; FortéBio). Response values were measured at 1800 seconds association time. Antibody dissociation rate (k_{off}) was measured by the Langmuir 1:1 binding model, with 30-600 seconds for the dissociation phase. The k_{off} for some serum samples could not be measured due to strong binding, in this case k_{off} were extrapolated to 2×10⁻⁵ (detectable dissociation from 0-1200 seconds for 5% signal decrease). Avidity index was calculated as, avidity index=response/k_{off}. All samples were analyzed in duplicate, average values are shown.

e) Microneutralization Test

Anti-dengue antibody titers (against serotypes 1-4) in response to vaccination with TAK-003 were quantified by microneutralization test (MNT), as previously described.

f) Correlation Between Antibody Titers and Avidity Assay Parameters

Post-vaccination avidity assay parameters of response, k_{off} and avidity for each serotype in 24 baseline seronegative and 19 seropositive groups of DEN-203 data were analyzed for correlation with MNT titers. Further correlation analyses were performed using data sets stratified by strength of binding (k_{off}) to DENV VLPs. Correlations were analyzed between avidity indices and neutralizing antibody titers for each stratified data set: low Log 10 k_{off}, middle Log 10 k_{off} and high Log 10 k_{off}. Response values under the limit of detection (LoD) of the assay were removed from correlation analysis.

g) Statistical Analysis

All data were analyzed by GraphPad Prism™ software (version 8.0.0; GraphPad Software Inc., San Diego, CA, USA).

2. Results

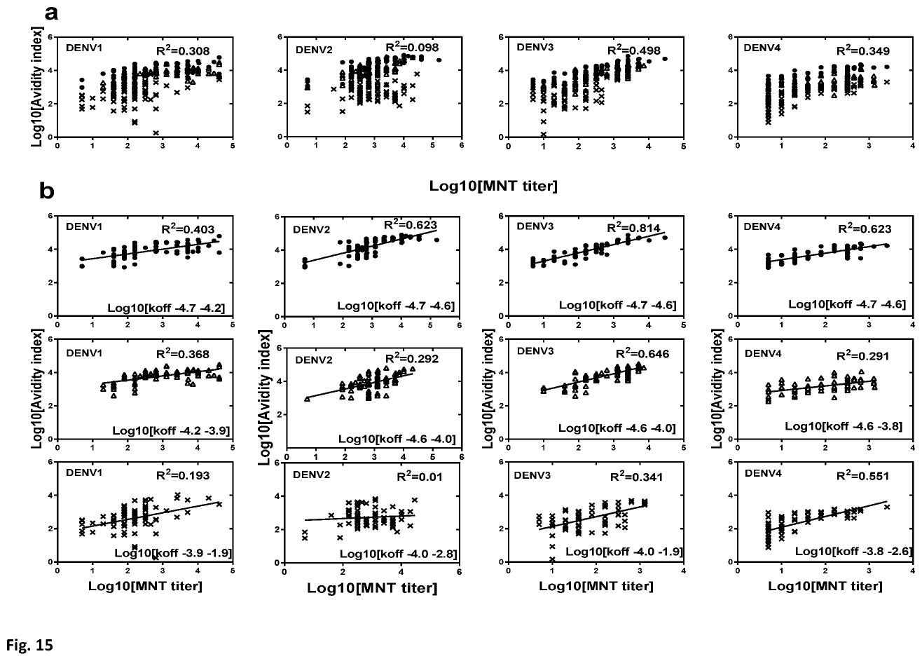

First, the degree of correlation between neutralizing antibody titers and avidity index parameters in DEN-203 study sera was assessed. When combining data for 43 subjects and study days for each DENV serotype, little to no correlation between MNT titer and avidity index was found [DENV1 R²=0.308, DENV2 R²=0.098, DENV3 R²=0.498, DENV4 R²=0.349] (FIG. 15a).

Next, the degree of correlation between neutralizing antibody titer and avidity index in samples with high, medium and low k_{off} values, representing sera with low, medium and high degree of polyclonal antibody affinity maturation was analyzed. For all serotypes, the degree of correlation between MNT and avidity index was lowest among samples with high k_{off} values (less affinity matured) and highest in samples with lower k_{off} values (more affinity matured) [e.g. DENV2 Log 10[k_{off}]: −4.7-−2.8: R²=0.098, Log 10[k_{off}]: −4.7-−4.6: R²=0.678, Log 10[k_{off}]: −4.6-−4.0: R2=0.292, Log 10[k_{off}]−4.0-−2.8: R²=0.010 (FIG. 15b and Table 10). This finding suggests a relationship between high affinity antibodies and neutralizing activity.

TABLE 10
Correlation parameters between avidity index
and MNT titer for k_{off} divided subjects.

| | | | | Log10[Avidity index] vs | |
|---|---|---|---|---|---|
| | Log10[k_{off}] | | | Log10[MNT titer] | |
| Serotype | range | | N | R square | Equation |
| DENV1 | −4.7 | −1.9 | 225 | 0.308 | Y = 0.459 X + 2.240 |
| | −4.7 | −4.2 | 75 | 0.403 | Y = 0.286 X + 3.150 |
| | −4.2 | −3.9 | 74 | 0.368 | Y = 0.254 X + 3.033 |
| | −3.9 | −1.9 | 76 | 0.193 | Y = 0.398 X + 1.756 |
| DENV2 | −4.7 | −2.8 | 230 | 0.098 | Y = 0.325 X + 2.668 |
| | −4.7 | −4.6 | 77 | 0.623 | Y = 0.426 X + 2.960 |
| | −4.6 | −4.0 | 77 | 0.292 | Y = 0.396 X + 2.749 |
| | −4.0 | −2.8 | 76 | 0.010 | Y = 0.076 X + 2.508 |
| DENV3 | −4.7 | −1.9 | 223 | 0.498 | Y = 0.657 X + 1.956 |
| | −4.7 | −4.6 | 75 | 0.814 | Y = 0.498 X + 2.795 |
| | −4.6 | −4.0 | 75 | 0.646 | Y = 0.497 X + 2.435 |
| | −4.0 | −1.9 | 73 | 0.341 | Y = 0.586 X + 1.545 |
| DENV4 | −4.7 | −2.6 | 197 | 0.349 | Y = 0.547 X + 2.128 |
| | −4.7 | −4.6 | 66 | 0.623 | Y = 0.392 X + 2.983 |
| | −4.6 | −3.8 | 65 | 0.291 | Y = 0.279 X + 2.638 |
| | −3.8 | −2.6 | 66 | 0.551 | Y = 0.643 X + 1.446 |

### Example 10: Coupling of Flavivirus Antigens to Microspheres

Microspheres used for coupling were MagPlex® microspheres (Luminex Corporation, Austin, Texas). MagPlex® microspheres are superparamagnetic polystyrene microspheres with surface carboxylic acid groups. The microspheres were delivered in a volume of 4 to 4.1 mL with an average concentration of 1.2 to 1.3×10⁷ microspheres per mL (microspheres/mL). MagPlex® microspheres are available in several unique regions, i.e. the microspheres comprise one or more fluorescent dyes having a defined emission signal (the detectable label) in order to distinguish the microspheres from microspheres of other unique regions. As the coupling mechanism involving the surface carboxyl groups is independent of the specific feature of the microspheres, MagPlex® microspheres of different unique regions may be exchanged according to variations in experimental set-ups.

DENV antigens for coupling to microspheres were DENV1 VLP (0.46 mg/mL liquid stock in 10 mM sodium phosphate, 20 mM sodium citrate, 154 mM sodium chloride pH 7.4; The Native Antigen Company, Product Code: DENV1-VLP-500, Batch No. 19040109), DENV2 VLP (0.52 mg/mL liquid stock in 10 mM sodium phosphate, mM sodium citrate, 154 mM sodium chloride pH 7.4; The Native Antigen Company, Product Code: DENV2-VLP-500, Batch No. 19040816), DENV3 VLP (0.72 mg/mL liquid stock in 10 mM sodium phosphate, 20 mM sodium citrate, 154 mM sodium chloride pH 7.4; The Native Antigen Company, Product Code: DENV3-VLP-500, Batch No. 18111415), and DENV4 VLP (0.53 mg/mL liquid stock in Dulbecco's phosphate-buffered saline (DPBS) pH 7.4, 30% sucrose; The Native Antigen Company, Product Code: DENV4-VLP-500, Batch No. 19061911).

DENV1-4 VLPs are consisting of DENV prM, M, and E protein produced in human embryonic kidney (HEK 293) cells. For production of DENV1-4 VLPs, the C-terminal 20% of DENV E protein were replaced by the corresponding Japanese encephalitis virus (JEV) SA-14 sequence (EMBL-EBI accession No: M55506.1; E protein amino acids 399-497 (DENV1 VLP), 397-495 (DENV2 VLP), 399-492 (DENV3 VLP), 400-495 (DENV4 VLP)). The replaced sequence corresponds to the transmembrane and intraparticle portion of the protein. DENV1 VLP was produced using the sequence from strain Puerto Rico/US/BID-Y853/1998 (GenBank accession No. EU482592.1, Uniprot No. B1PNU3). DENV2 VLP was produced using the sequence from strain Thailand/16681/84 (EMBL-EBI accession No: U87411.1, Uniprot No. P29990.1). DENV3 VLP was produced using the sequence from strain Sri Lanka D3/H/IMTSSA-SRI/2000/1266 (GenBank accession No. AY099336.1, Uniprot No. Q6YMS4.1). DENV4 VLP was produced using the sequence from strain Dominica/814669/1981 (EMBL-EBI accession No: AF326825.1, Uniprot No. P09866.2).

ZIKV VLPs (The Native Antigen Company, Product Code: ZIKV-VLP) comprise prM, M, and E protein of ZIKV strain Z1106033 isolated in Suriname (Asian genotype; Enfissi et al., Lancet 2016, 387(10015):227-228; GenBank Accession No. KU312312.1 and ALX35659.1), include amino acids 123-819 and are produced in HEK 293 cells.

DENV1-4 NS1 (The Native Antigen Company, Product Code: DENVX4-NS1) and ZIKV NS1 (The Native Antigen Company, Product Code: ZIKVSU-NS1) are produced in human embryonic kidney (HEK 293) cells. DENV1 NS1 was produced using the sequence from strain Nauru/Western Pacific/1974 (GenBank accession No. AY145121). DENV2 NS1 was produced using the sequence from strain Thailand/16681/84 (EMBL-EBI accession No: U87411.1, Uniprot No. P29990.1). DENV3 NS1 was produced using the sequence from strain Sri Lanka D3/H/IMTSSA-SRI/2000/1266 (GenBank accession No. AY099336.1, Uniprot No. Q6YMS4.1). DENV4 NS1 was produced using the sequence from strain Dominica/814669/1981 (EMBL-EBI accession No: AF326825.1, Uniprot No. P09866.2). ZIKV NS1 was produced using the sequence (amino acids 795-1146) from strain Suriname Z110603 (GenBank Accession No. KU312312.1 and ALX35659.1) and buffered in PBS, pH 7.4.

VLPs are a desirable reagent for coupling to the microspheres because of their ease of manufacture, antigenic fidelity, and lack of safety concerns. Further, for evaluation of complement fixing antibodies against all DENV serotypes produced upon vaccination with live attenuated viruses comprising E and prM structural proteins, VLPs are favorable as they are a good surrogate for the whole virion. Moreover, antibodies against structural antigens i.e. E and prM protein can be detected by the application of one single antigen (the VLP).

Different microspheres comprising one or more fluorescent dyes having a specific emission signal (different unique regions) were applied for coupling of the different antigens to provide the possibility to distinguish the microspheres according to their coupled antigens when analyzed within one sample (capability to multi-plex). For example, DENV1 VLP was coupled to a set of MagPlex® microspheres of region 76, DENV2 VLP was coupled to a set of MagPlex® microspheres of region 14, DENV3 VLP was coupled to a set of MagPlex® microspheres of region 25, and DENV4 VLP was coupled to a set of MagPlex® microspheres of region 33. DENV1 NS1 was coupled to a set of MagPlex® microspheres of region 45, DENV2 NS1 was coupled to a set of MagPlex® microspheres of region 65, DENV3 NS1 was coupled to a set of MagPlex® microspheres of region 66, and DENV4 NS1 was coupled to a set of MagPlex® microspheres of region 67. ZIKV VLP was coupled to a set of MagPlex® microspheres of region 47, ZIKV NS1 was coupled to a set of MagPlex microspheres of region 36.

Coupling of Flavivirus Antigens to Microspheres

The uncoupled stocks of MagPlex® microsphere suspensions (1.2 to 1.3×10⁷ microspheres/mL, Luminex Corporation, Austin, Texas) were resuspended by vortexing (30 sec) and 12.5×10⁶ microspheres of each stock were transferred to 5 mL microcentrifuge tubes and placed into a 5 mL tubes magnetic separator (Life Technologies). Separation of the microspheres from the suspension occurred for 30-60 sec. Supernatant was carefully removed without disrupting the microsphere pellet while the tubes were still positioned in the magnetic separator. Afterwards, the tubes were removed from the magnetic separator and the microspheres were resuspended in 500 μL distilled H₂O (dH₂O) by vortexing and sonication for approximately 20 sec. The tubes were again placed into the magnetic separator and separation of the microspheres from the suspension occurred for 30-60 sec. Supernatant was removed without disrupting the microsphere pellet while the tubes were still positioned in the magnetic separator. The microspheres were resuspended in 400 μl of activation buffer (0.1 M sodium phosphate (monobasic) pH 6.2) and mixed by vortexing and sonication for 20 sec. Then, 50 μL of 50 mg/mL N-hydroxysulfosuccinimide (Sulfo-NHS; 50 mg of Sulfo-NHS in 1000 μL of dH₂O; Thermo Fisher Scientific) were added to each microsphere tube and gentle mixing was carried out by vortexing (5 sec). Further, 50 μL of 50 mg/mL 1-Ethyl-3-(3-dimethylaminopropyl)carbodiimide (EDC; 10 mg EDC in 200 μL of dH₂O; Thermo Fisher Scientific) were added to each microsphere tube and gentle mixing was carried out by vortexing (5 sec). Samples were incubated for 20 mM at room temperature with gentle mixing by vortexing after 10 min. The tubes were placed into the magnetic separator and separation of the microspheres from the suspension occurred for 30-60 sec. Supernatant was removed without disrupting the microsphere pellet while the tubes were still positioned in the magnetic separator. The tubes were removed from the magnetic separator and the microspheres were resuspended in 500 μL of 50 mM 2-(N-morpholino)ethanesulfonic acid (MES) buffer at pH 6.00 for DENV1-4 VLP, DENV1-4 NS1 proteins, and ZIKV NS1 protein (Boston Bioproducts, Cat. No. BBMS-60, Lot. No. F03K118) or at pH 7.00 for ZIKV VLP by vortexing and sonication for 20 sec. The tubes were placed into the magnetic separator and separation of the microspheres from the suspension occurred for 30-60 sec. Supernatant was removed without disrupting the microsphere pellet while the tubes were still positioned in the magnetic separator. The tubes were removed from the magnetic separator and the microspheres were resuspended in 500 μL of corresponding 50 mM MES buffer by vortexing and sonication for 20 sec. The tubes were placed into the magnetic separator and separation of the microspheres from the suspension occurred for 30-60 sec. Supernatant was removed without disrupting the microsphere pellet while the tubes were still positioned in the magnetic separator.

Afterwards, 1000 μL of a solution of each one of the DENV1-4 VLPs, DENV1-4 NS1 proteins, ZIKV VLP and ZIKV NS1 (diluted in 50 mM MES buffer at pH 6.0 for all NS1 proteins (DENV and ZIKV), as well as DENV1-4 VLPs and at pH 7.0 for ZIKV VLP) were transferred to a different 5 mL tube containing the activated microspheres to result in a ratio of 5 μg antigen per 10⁶ microspheres in a total volume of 1000 μL. The mixture was vortex for 20 sec. For coupling, samples were incubated for 2 hours under rotation at room temperature. The tubes were placed into the magnetic separator and separation of the microspheres from the suspension occurred for 30-60 sec. Supernatant was removed without disrupting the microsphere pellet while the tubes were still positioned in the magnetic separator. The tubes were removed from the magnetic separator and the microspheres were resuspended in 1 mL of 1% BSA in 1-fold PBS pH 7.4 by vortexing for approximately 20 sec. The tubes were placed into the magnetic separator and separation of the microspheres from the suspension occurred for 30-60 sec. Supernatant was removed without disrupting the microsphere pellet while the tubes were still positioned in the magnetic separator. The tubes were removed from the magnetic separator and the microspheres were resuspended in 1 mL of 1% BSA in 1-fold PBS pH 7.4 by vortexing for approximately 20 sec. The tubes were placed into the magnetic separator and separation of the microspheres from the suspension occurred for 30-60 sec. Supernatant was removed without disrupting the microsphere pellet while the tubes were still positioned in the magnetic separator. The tubes were removed from the magnetic separator and the microspheres were resuspended in 2000 μL of 1% BSA in 1-fold PBS pH 7.4 by vortexing for approximately 20 sec. The microspheres were kept in the 2.0 mL tubes. In order to count the microspheres recovered after the coupling reaction, the microsphere suspension was diluted 2-fold in 1% BSA in 1-fold PBS pH 7.4 (e.g. 15 μL microsphere suspension diluted with 15 μL of 1% BSA in 1-fold PBS pH 7.4). The number of microspheres recovered after the coupling reaction was determined using an automated cell counter (Countes II, Thermo Fisher Scientific, Cat. No. AMQAX1000) by correlating the determined “dead cells” concentration provided by the cell counter to the microspheres. The coupled microspheres were stored at 2-8° C. in the dark (blocking step) separately for each antigen. Previous to use, the coupled microspheres were allowed to pre-warm for at least 30 mM at room temperature.

Optimization of Coupling Conditions

As the coupling efficiency, as well as the integrity of the antigen after the coupling procedure is dependent on the buffer used, different buffer conditions were evaluated for coupling of the VLPs and NS1 proteins to the microspheres. Coupling carried out as described above, except that the buffer was varied. Optimization of the coupling procedure is important in order to ensure that the three-dimensional structure of the antigen is not disturbed. The buffer conditions may vary dependent on the type of antigen used.

Different buffer conditions (50 mM MES at pH 5.00, 6.00, and 7.00) for coupling were examined and the signal to noise ratio was evaluated in a DENV-quadruplex complement-fixing assay set-up essentially as described under Example 2. Therefore, a negative control, i.e. a serum sample lacking DENV antibodies, and a control sample, i.e. a plasma sample derived from a human subject living in DENV endemic areas in Colombia with high amounts of anti-DENV complement fixing Abs were tested. The assay shows, that a pH of 6.00 results in the highest fluorescence signal from the high control sample. In addition, also a pH of 7.00 shows a high signal to noise ratio. In contrast, a lower pH of 5.00 did not result in high signals indicating that the pH is not suitable for VLP coupling. For comparison, when coupling single proteins such as NS1, the optimum pH values seem to be different. While for the VLP a coupling pH of 7.00 results in high fluorescence signals, for the NS1 protein the same pH does solely result in low fluorescence signals, indicating significant differences in coupling efficacies depending on the size, as well as three-dimensional structure of the antigen. Satisfyingly, no background signal was observed from the negative control sample neither for the VLP, nor for the NS1 protein independent of the buffer pH.

As described above, for routine coupling of DENV1-4 VLPs and DENV1-4 NS1 50 mM MES at pH 6.00 was applied. Of note, the optimum coupling pH for ZIKV VLP was 50 mM MES at pH 7.00 indicating that also virus-specific effects have an impact on the coupling efficiency.

### Example 11: Evaluation of a DENV-Quadruplex Complement-Fixing Assay Set-Up

The DENV1-4 VLP-coupled microspheres of Example 10 were applied to develop a DENV-quadruplex complement-fixing assay set-up as described in the following.

For evaluation of the complement-fixing assay, a reference sample, as well as control samples were analyzed. The reference sample consists of pooled plasma samples from human subjects living in DENV endemic areas in Colombia with high levels of anti-DENV complement-fixing Abs (ABO Pharmaceuticals, Lot. VBU-01140-148). Control samples comprised plasma derived from human subjects living in DENV endemic areas in Colombia, including high, medium, and low amounts of anti-DENV complement fixing Abs (ABO Pharmaceuticals, Lot. No. PARS_82 (high control), Lot. No. PARS_96 (medium control), Lot. No. VBU-01140-189 (low control)). In addition, a negative control sample was included consisting of serum lacking any anti-DENV Abs (Bioreclamation, Lot.-No. BRH1140253). In general, the assay can be performed with either matrices i.e. serum or plasma samples. All samples were stored at −80° C. prior to use. The samples were thawed and heat inactivated in a 56±1° C. water bath (Thermo Fisher, Isotemp 210, Cat.-No. 15-462-10Q) for 30±5 minutes prior to testing. Heat-inactivation is important to denature the temperature sensitive complement proteins within the sample and thereby to avoid assay interference.

In a next step, the DENV1-4 VLP-coupled microspheres of Example 10 were vortexed gently to break up clumping of the microspheres and ensure a uniform suspension. The microspheres were combined by dilution in assay buffer to result in a final concentration of 25 microspheres/μL for each DENV serotype and vortexed gently. Assay buffer consisted of phosphate buffered saline (PBS) with 1% bovine serum albumin (BSA), diluted from a 10% stock (Fisher Scientific, Cat-No. 37525) and was stored at 2-8° C. for up to one month. The assay buffer was allowed to pre-warm for at least 30 minutes at room temperature prior to dilution of the microspheres. 50 μL of the microsphere suspension containing all four DENV serotype antigens were pipetted per well into a 96-well polystyrene microplate (solid black flat bottom plate, in the following referred to as “assay plate”; Corning, Cat.-No. 3915) resulting in 1250 microspheres per DENV-serotype per well. The plate was sealed with a foil plate seal (ThermoFisher, Cat.-No. AB0558) and stored at room temperature until the samples were diluted.

Heat-inactivated samples were serially diluted (8 dilutions final) using assay buffer pre-warmed to room temperature at least 30 minutes before testing. 50 μL per sample dilution were transferred into the assay plate per well to the microspheres in duplicates. Sample and microsphere suspension were mixed thoroughly by pipetting up and down 3-5 times. Next, the plate was sealed with a foil plate seal and incubated for 60±5 min at room temperature on a plate shaker (Heidolph, Titramax 1000, Cat.-No. 544-12200-00) at 600 rpm.

Afterwards, the plate was washed with wash buffer (PBS with 0.05% Tween-20) using the Luminex Flat 96 Mag setting on a plate washer (BioTek ELx405, Cat.-No. 7100745S). After decanting of residual wash buffer, 50 μL/well of purified human C1q at a concentration of 4.0 μg/mL in assay buffer were added. Human C1q (Quidel, Cat.-No. A400, Lot.-No. 142550) is purified (>95%) from plasma, reconstituted with 40% (v/v) glycerol in 4-(2-hydroxyethyl)-1-piperazineethanesulfonic acid (HEPES) buffer at 1 mg/mL and stored at −80° C. prior to use. After C1q addition, the plate was sealed with a foil plate seal and incubated for 30±5 min at room temperature on a plate shaker at 600 rpm. By using purified C1q the exact amount of C1q applied in the assay can be controlled. In contrast, by using complement-component human serum the C1q concentration in the serum could vary between different lots of the serum. In addition, also the binding of C1q could be interfered by other serum components. Thus, the use of purified C1q improves reproducibility of the assay.

After incubation with C1q, the plate was washed with wash buffer as described above. After decanting of residual wash buffer, a pre-reporter Ab, i.e. sheep IgG anti-human C1q was added 50 μL/well at a 6.4 μg/mL dilution in assay buffer. Polyclonal purified sheep IgG anti-human C1q Ab (Bio-Rad, Cat.-No. AHP033, Lot.-No. 148553) recognizes human C1q. The Ab is solubilized in glycine buffer saline from manufacturer to 5.1 mg/mL and stored at 4° C. prior to use. After Ab addition, the plate was sealed with a foil plate seal and incubated for 30±5 min at room temperature on a plate shaker at 600 rpm.

After incubation with anti-C1q Ab, the plate was washed with wash buffer as described above. After decanting of residual wash buffer, a reporter Ab, i.e. PE-conjugated donkey IgG anti-sheep IgG was added 50 μL/well at a 10 μg/mL dilution in assay buffer. R-Phycoerythrin F(ab′)₂ fragment donkey anti-sheep IgG Ab (Jackson ImmunoResearch, Cat.-No. 713-116-147) is sold freeze-dried and was reconstituted with 1 mL deionized water according to the manufacturer to result in a 500 μg/mL solution. After Ab addition, the plate was sealed with a foil plate seal and incubated for 30±5 min at room temperature on a plate shaker at 600 rpm.

Finally, the plate was washed with wash buffer as described above. After decanting of residual wash buffer, 100 μL/well of assay buffer were added to the plate. The plate was covered with foil plate seal and shaken at 600 rpm for 5 min in order to resuspend the microspheres prior to read. At this point, the plate may also be stored at 2-8° C. overnight for analysis on the following day. If refrigerated overnight, the plate was shaken at room temperature for at least 30 min at 600 rpm.

The plate was analyzed in a MAGPIX® Luminex plate reader with the xPONENT 4.2 software (Luminex Corp., Cat.-No. MAGPIX-XPONENT). The Luminex acquisition mode was set to 50 μL sample volume and 50 microspheres of each DENV serotype per well. The number of microspheres determined by the acquisition mode specifies that the Luminex reader needs to acquire at least 50 microspheres of each DENV serotype per well in order to determine the representative (statistically) mean signal of all microspheres of each DENV serotype per well.

For data analysis, each DENV serotype antigen is independently evaluated resulting in four standalone immunoassays. Data were analyzed and plotted using GraphPad Prism 8 version 8.1.0 (GraphPad Software, Inc). Mean Fluorescent Intensity (MFI; MFI values are the raw data reported by the MAGPIX® reader) values were plotted in dependence of log₁₀-transformed serum dilutions (e.g. 5-fold dilution resulting in log₁₀(5)=0.7). Sigmoidal fitting was performed according to a dose-response curve (Sigmoidal, 4PL, X=Log(concentration)). The equation used for the non-linear regression was “log(agonist) vs. response—Variable slope”. The MFI signal threshold of the reference sample equivalent to the EC₂₅ concentration was calculated by subtracting the estimated bottom from top signals and multiplying the result by 0.25 (signal equivalent to 25% of effective concentration). The MFI signal threshold was then interpolated on the 4PL curves of both the reference sample and the control samples (as well as corresponding test samples) to determine the sample dilution equivalent to the EC₂₅ signal of the reference (interpolated dilution). The interpolated dilutions are then divided by the interpolated dilution calculated in the reference to result in relative dilutions. Further, relative dilutions were multiplied by a constant (EC50 of the reference against each DENV serotype) for the corresponding DENV serotype (i.e. 468 for DENV-1 VLP, 345 for DENV-2 VLP, 369 for DENV-3 VLP and 257 for DENV-4 VLP) to result in the complement-fixing antibody titers in the samples in EU/mL. The EU/mL concentration is a relative arbitrary concentration based on the levels of complement-fixing antibodies found in the reference sample.

Signals Resulting from Different DENV VLPs

MFI values of the different sample dilutions and corresponding dose-response curve fits of reference and control samples have been obtained. Satisfyingly, for all four DENV VLPs MFI signals increased for all the samples except for the negative control with decreasing dilution factor. In addition, MFI signals increased from low to high titer control samples. Moreover, negative control serum did result in MFI signals close to zero independent of the dilution.

Limit of Detection (LOD)

The LOD was determined by serial dilution of the reference sample in assay buffer and determination of the lowest complement-fixing antibody concentration for which the relative error (% RE) is above 25% in one independent run using duplicates per dilution. In summary, the LOD ranged from 0.46 EU/mL for DENV1 VLP to 0.72 EU/mL for DENV3 VLP.

Lower Limit of Quantification (LLOQ)

The LLOQ was determined by serial dilution of the reference sample in human IgG-depleted serum and determination of the lowest complement-fixing antibody concentration in which the relative error (% RE) is above 25% in five independent runs. The LLOQ for all DENV VLPs was determined to be 3.00 EU/mL.

Assay Linearity

Assay linearity was evaluated by plotting the median value of the obtained concentration of the five independent runs performed for determination of the LLOQ for each reference sample dilution and DENV VLP against the expected concentration. The obtained and expected concentrations correlated well and in a linear manner with slopes close to one.

Assay Precision

To evaluate the assay precision high, medium and low control samples were tested five times per run. In total, two different operators performed each two runs. Complete sigmoidal curves are exemplarily shown for one run and the DENV2 VLP antigen. Satisfyingly, fluorescence signals for the five replicates were highly similar independent of the signal intensity and sample type. In general, complement-fixing Ab titers were highly similar independent of the run, the control sample, the DENV VLP, and the operator.

For determination of intra-assay precision, the percent coefficient of variation (% CV) of the complement-fixing Ab concentration was calculated for each sample within each run. For determination of inter-assay precision, the percent coefficient of variation (% CV) of the complement-fixing Ab concentration was calculated in between the runs.

Intra-assay precision was consistently below 20% for all control samples and DENV VLPs. Inter-assay precision was below 20% for all control samples and DENV VLPs, except for the DENV1 VLP signals from the low control sample, which was solely slightly above 20%.

In conclusion, the DENV-quadruplex complement-fixing assay was successfully set-up and validated.

### Example 12: Detailed Characterization of Immune Responses to a Live-Attenuated Tetravalent Dengue Vaccine in Adults from the United States

Methods

The live attenuated tetravalent dengue vaccine candidate (TAK-003) from the company Takeda has been administered in a phase 3 clinical trial conducted in the United States. Serum from baseline seronegative TAK-003 recipients (N=48) aged 18-60 years old was collected pre-vaccination, 1 month and 6 months post 2n d dose for analyses, and assayed for magnitude and quality of humoral antibody response against the viral structural and non-structural proteins, including anti-dengue NS1 IgG response, dengue total binding IgG response, avidity of anti-dengue IgG response and complement fixing Ab response. A subset of these samples were also analyzed for type-specificity of neutralizing antibody response.

Results

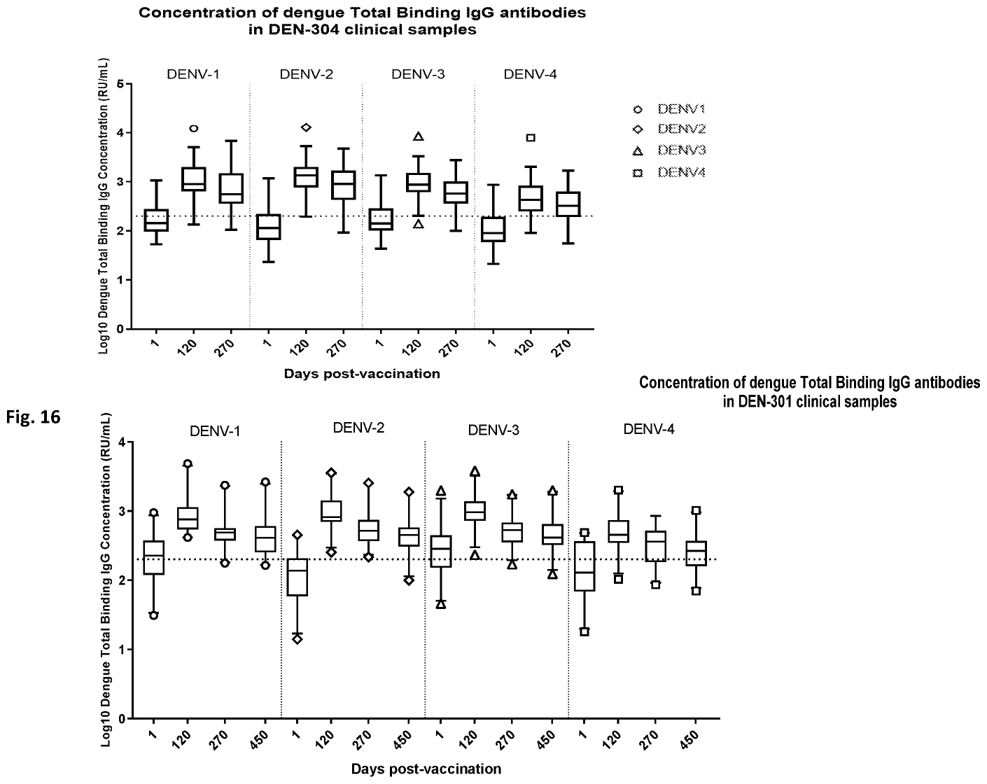

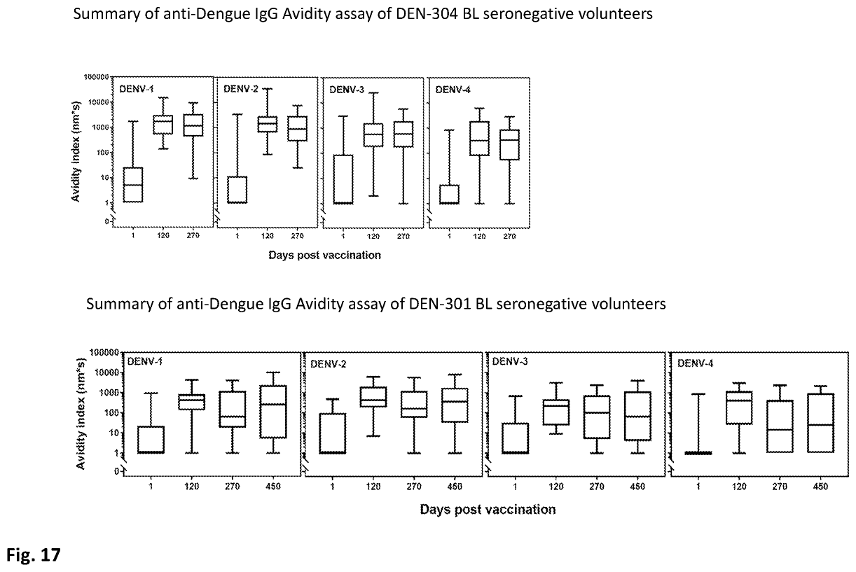

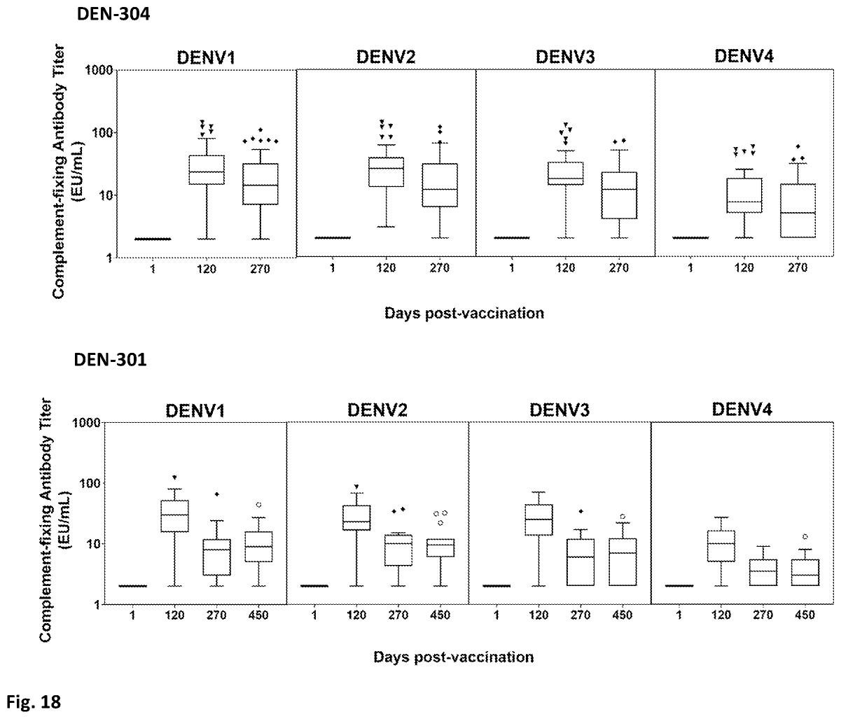

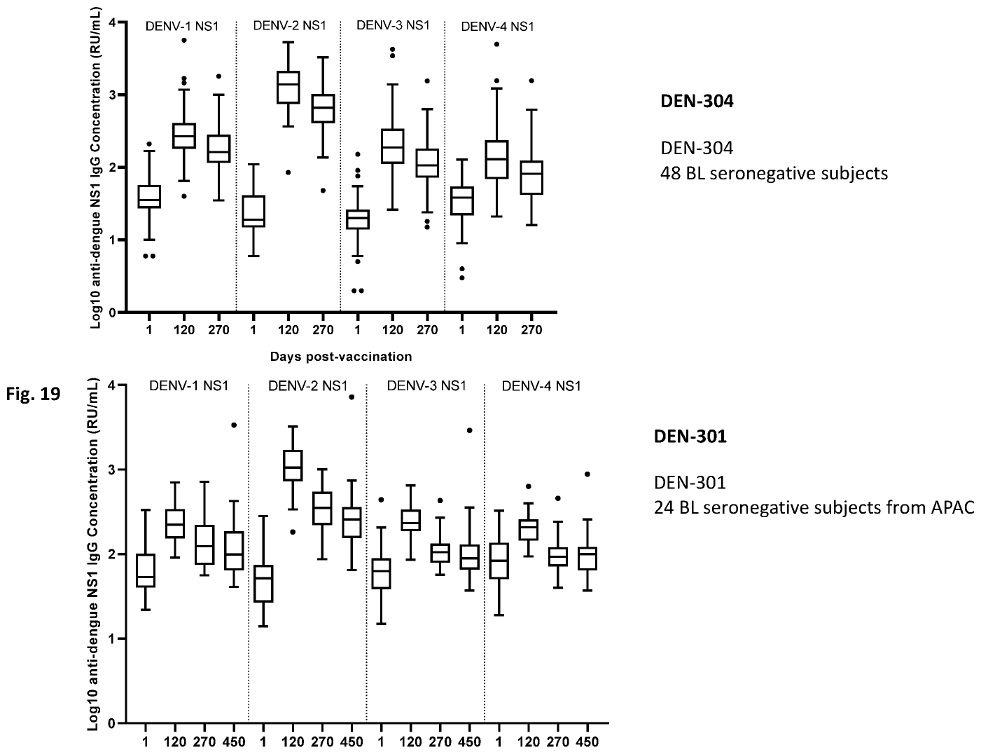

The results are shown in FIGS. 16 to 19. They show that TAK-003 elicits measurable, sustained and affinity matured binding IgG responses against all 4 dengue serotypes. The functional binding antibody responses include cross-reactive and type-specific neutralizing antibodies to all serotypes tested (DENV-1, 3, and 4), and tetravalent complement fixing antibodies. TAK-003 also elicits functional

DENV-2 NS1-specific antibodies that are cross-reactive with NS1 from DENV-1, 3, and 4. In conclusion, in adults, TAK-003 elicits multifunctional antiviral immune responses directed against the viral structural and nonstructural proteins. These responses are comparable to the vaccine-elicited immune responses in children and adolescents, and provide supportive evidence for the use of TAK-003 across a broad age range and as a travel vaccine.

### Example 13: Anti-Dengue NS1 Antibody Levels Induced by TAK-003

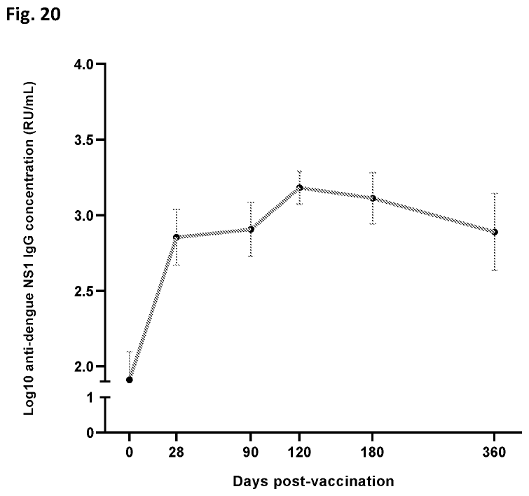

FIG. 20 shows the concentration of anti-dengue NS1 antibodies induced by vaccination with the vaccine TAK-003 over time. The blood samples have been removed from the vaccinated individuals at different time points during the clinical trial DEN-203. The NS1 antibody concentration was describes as outlined above. As can be seen, the anti-dengue NS1 antibodies concentration observed at day 28 is essentially stable until day 360. This is of particular relevance, since anti-dengue

NS1 antibodies are known to confer protection against dengue disease.

### Example 14: Correlation Analysis

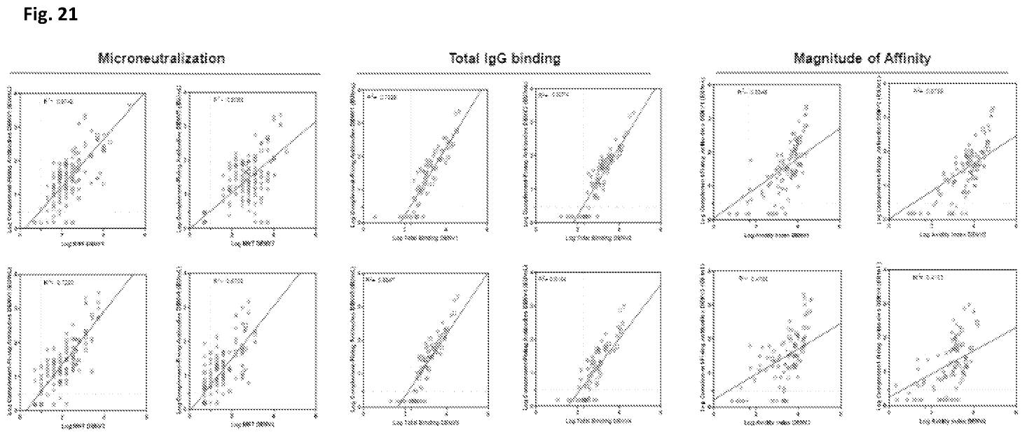

FIG. 21 shows a correlation analysis obtained by performing linear regression of the log 10-transformed concentration between anti-dengue complement-fixing antibody levels and microneutralization (MNT value), total IgG binding and magnitude of affinity, respectively, after vaccination of individuals with TAK-003. Total binding IgG ELISA was carried out as described above. Avidity index was determined as described above. The correlation analysis was performed with the statistical software JMP version 15.2 (SAS Institute).

Complement-fixing antibodies were strongly correlated with the MNT value and the total IgG binding. This is particularly important, since complement-fixing antibodies against dengue antigens are known to be protective against dengue disease.

A correlation to a lesser extent of the complement-fixing antibodies and the magnitude of affinity of the antibodies was also observed.

1. A method for determining affinity, binding kinetics and/or concentration of an antibody or of an antibody mixture specific for a virus comprising the following steps:
a) providing a virus-like particle (VLP) attached to a biosensor, wherein said VLP comprises structural proteins from said virus;
b) contacting the VLP attached to the biosensor with a first solution containing the antibody or antibody mixture specific for the virus such that the antibody or antibody mixture binds to the VLP attached to the biosensor and measuring the association of the binding complex;
c) contacting the VLP attached to the biosensor having bound the antibody or antibody mixture with a second solution lacking the antibody or antibody mixture such that the antibody or antibody mixture dissociates from the VLP attached to the biosensor and measuring the dissociation of the binding complex, wherein the measuring in steps b) and c) are performed by surface plasmon resonance (SPR) or biolayer interferometry (BLI); and
d) calculating the affinity, binding kinetics and/or concentration of the antibody or the antibody mixture specific for the virus from the measurement data in steps b) and c).

2. The method according to claim 1, wherein steps b) and c) are performed by BLI.

3. The method of claim 1 or 2, wherein the virus is a flavivirus or a calicivirus and the VLP is derived from said flavivirus or said calicivirus.

4. The method of claim 3, wherein the virus is selected from Dengue virus, Japanese encephalitis virus, Tick-borne encephalitis virus, West Nile virus, Yellow fever, Zika virus and Norovirus, preferably, the virus is Dengue virus, Zika virus or Norovirus.

5. The method of claim 4, wherein the virus is selected from any of the Dengue virus subtypes DENV-1, DENV-2, DENV-3 and DENV-4.

6. The method of any one of claims 1 to 5, wherein the VLP is attached to the biosensor by any of the following:
i) a pair of binding molecules capable of specifically binding to each other, wherein the first binding molecule is linked to the VLP and the second binding molecule is attached to the surface of the biosensor; and/or
ii) a covalent linkage of the VLP to a capture reagent attached to the biosensor.

7. The method of claim 6, wherein the pair of binding molecules is selected from biotin/streptavidin; ligand/receptor; antigen/antibody; antibody/Protein A or Protein G; sugar/lectin; his-tag/Ni and sense/antisense oligonucleotides, preferably the pair of binding molecules is biotin/streptavidin.

8. The method of claim 6 or 7, wherein the VLP is biotinylated and the biosensor has streptavidin attached to its surface.

9. The method claim 6 or 7, wherein the VLP is covalently linked to a biosensor having an amine-reactive surface.

10. The method of any one of claims 1 to 9, wherein steps b) and c) comprise the following steps:
i) contacting the VLP attached to the biosensor with a composition containing the antibody or the antibody mixture under conditions allowing association of the VLP with the antibody or antibody mixture; and
ii) removing the VLP attached to the biosensor from the composition containing the antibody or the antibody mixture and incubating the VLP attached to the biosensor under conditions allowing the dissociation of the VLP from the antibody or antibody mixture.

11. A method for determining affinity, binding kinetics and/or concentration of an antibody or of an antibody mixture specific for a virus comprising the following steps:
a) providing a live virus or an inactivated virus attached to a biosensor;
b) contacting the live virus or inactivated virus attached to the biosensor with a first solution containing the antibody or antibody mixture specific for the virus such that the antibody or antibody mixture binds to the live virus or inactivated virus attached to the biosensor and measuring the association of the binding complex;
c) contacting the live virus or inactivated virus attached to the biosensor having bound the antibody or antibody mixture with a second solution lacking the antibody or antibody mixture such that the antibody or antibody mixture dissociates from the live virus or inactivated virus attached to the biosensor and measuring the dissociation of the binding complex, wherein the measuring in steps b) and c) are performed by SPR or BLI; and
d) calculating the affinity, binding kinetics and/or concentration of the antibody or the antibody mixture specific for the virus.

12. The method according to claim 11, wherein steps b) and c) are performed by BLI.

13. The method of claim 10, wherein said live virus or said inactivated virus is a flavivirus or a calicivirus.

14. The method of claim 13, wherein the virus is selected from Dengue virus, Japanese encephalitis virus, Tick-borne encephalitis virus, West Nile virus, Yellow fever, Zika virus and Norovirus, preferably, the virus is Dengue virus, Zika virus or Norovirus.

15. The method of claim 14, wherein the virus is selected from any of the Dengue virus subtypes DENV-1, DENV-2, DENV-3 and DENV-4.

16. The method of claim any one of claims 11 to 15, wherein said live virus or said inactivated virus is attached to the biosensor by hydrophobic interaction of said live virus or said inactivated virus with a capture reagent linked to the surface of the biosensor.

17. The method of claim 16, wherein the capture reagent comprises aminopropylsilane.

18. The method of any one of claims 11 to 17, wherein steps b) and c) comprise the following steps:
i) contacting said live virus or said inactivated virus with a composition containing said antibody or said antibody mixture under conditions allowing association of said live virus or said inactivated virus with the antibody or antibody mixture; and
ii) removing said live virus or said inactivated virus from the composition containing said antibody or said antibody mixture and incubating said live virus or said inactivated virus under conditions allowing the dissociation of said live virus or said inactivated virus from said antibody or said antibody mixture.

19. The method of any one of claims 1 to 18, wherein said antibody mixture is a polyclonal mixture of antibodies from serum of a human subject immunized with the virus or a virus antigen thereof.

20. The method of claim 19, wherein said antibody or said antibody mixture is purified by affinity chromatography, preferably the affinity chromatography comprises the use of Protein A, Protein G, Protein A/G or Protein L affinity chromatography.

21. The method of any one of claims 1 to 20, wherein the method is used for determining the avidity of the antibody or antibody mixture for the virus, preferably the avidity index is determined.

22. A method for determining the avidity and/or affinity over time of an antibody or antibody mixture produced after immunization of a human subject with a virus vaccine comprising the following steps:
a) obtaining serum samples from said subject at different time points after immunization;
b) purifying the antibody or antibody mixture from the serum samples by affinity chromatography using Protein A Protein G, Protein A/G or Protein L;
c) determining the avidity and/or affinity of the antibodies for the virus as a function over time in accordance with the method of any one of claims 1 to 21.

23. The method according to claim 22, wherein the method determines the avidity index of the antibody or antibody mixture from serum samples obtained after different points of time after immunization.

24. The method according to claim 23, wherein the virus is selected from Dengue virus, Japanese encephalitis virus, Tick-borne encephalitis virus, West Nile virus, Yellow fever, Zika virus and Norovirus, preferably, the virus is Dengue virus, Zika virus or Norovirus.

25. The method according to any one of claims 22 to 24, wherein the virus vaccine is a tetravalent dengue virus composition comprising four live, attenuated dengue virus strains.

26. The method according to claim 25, wherein the four live, attenuated dengue virus strains are:
(i) a chimeric dengue serotype 2/1 strain, preferably the chimeric serotype 2/1 strain has the amino acid sequence set forth in SEQ ID NO: 2,
(ii) a dengue serotype 2 strain, preferably the serotype 2 strain has the amino acid sequence set forth in SEQ ID NO: 4,
(iii) a chimeric dengue serotype 2/3 strain, preferably the chimeric serotype 2/3 strain has the amino acid sequence set forth in SEQ ID NO: 6, and
(iv) a chimeric dengue serotype 2/4 strain, preferably the chimeric serotype 2/4 strain has the amino acid sequence set forth in SEQ ID NO: 8.

27. The method according to claim 25 or 26, wherein each one of the four live, attenuated dengue virus strains has attenuating mutations in the 5′-noncoding region (NCR) at nucleotide 57 from cytosine to thymine, in the NS1 gene at nucleotide 2579 from guanine to adenine resulting in an amino acid change at position 828 of the NS1 protein from glycine to asparagine, and in the NS3 gene at nucleotide 5270 from adenine to thymine resulting in an amino acid change at position 1725 of the NS3 protein from glutamine to valine.

28. A method of preparing a virus-like particle (VLP) attached to a biosensor suitable for SPR or BLI, wherein said VLP comprises structural proteins from said virus, wherein the method comprises attaching the VLP to the biosensor by any of the following:
i) a pair of binding molecules capable of specifically binding to each other, wherein the first binding molecule is linked to the VLP and the second binding molecule is attached to the surface of the biosensor; and/or
ii) a covalent linkage of the VLP to a capture reagent attached to the biosensor.

29. The method according to claim 28, wherein the virus is a flavivirus or a calicivirus, preferably the virus is selected from Dengue virus, Japanese encephalitis virus, Tick-borne encephalitis virus, West Nile virus, Yellow fever virus, Zika virus and Norovirus.

30. A VLP attached to a biosensor suitable for SPR or BLI obtainable by the method according to claims 28 to 29.

31. A method of preparing a live virus or an inactivated virus attached to a biosensor suitable for SPR or BLI, wherein the method comprises attaching said live virus or said inactivated virus to the biosensor by hydrophobic interaction of said live virus or said inactivated virus with a capture reagent linked to the surface of the biosensor.

32. The method according to claim 31, wherein the virus is a flavivirus or a calicivirus, preferably the virus is selected from Dengue virus, Japanese encephalitis virus, Tick-borne encephalitis virus, West Nile virus, Yellow fever virus, Zika virus and Norovirus.

33. A live virus or inactivated virus attached to a biosensor suitable for SPR or BLI obtainable by the method according to claim 31 or 32.

34. Method for characterizing the immune response of a subject to a tetravalent dengue virus composition administered to said subject, comprising performing with a serum sample from said subject the method according to any one of claims 1 to 27 and at least one other method selected from the group consisting of:
(a) a method to determine the level of neutralizing antibodies in said sample;
(b) a method comprising depleting antibodies against one dengue serotype from said sample followed by determining in the depleted sample the level of neutralizing antibodies against at least one serotype different from the dengue serotype used for depleting the antibodies, to detect the presence of type-specific and cross-reactive neutralizing antibodies in said sample;
(c) a method to determine the level of antibodies against a non-structural protein 1 of dengue virus in said sample;
(d) a method to determine the level of dengue-binding antibodies in said sample; and
(e) a method to determine the presence and/or amount of flavivirus-reactive complement-fixing antibodies in said sample.

35. Method according to claim 34, wherein the method of (a) is a neutralization assay.

36. Method according to claim 35, wherein the neutralization assay comprises the steps of:
(i) seeding cells from a dengue-susceptible cell line and culturing the cells for a culture period;
(ii) preparing serial dilutions of the serum sample;
(iii) separately mixing the serially diluted serum samples prepared in step (b) with dengue serotype 1, dengue serotype 2, dengue serotype 3 and dengue serotype 4 to obtain separate mixtures for each dengue serotype and incubating the separate mixtures;
(iv) adding the separate mixtures prepared in step (c) to the cells seeded and cultured in step (a) and incubating the cells with the separate mixtures;
(v) providing an overlay for the cells incubated in step (d) and incubating the cells for an incubation period;
(vi) determining the number of plaques in each well and comparing the number of plaques in each well to an non-neutralized control to determine the level of neutralizing antibodies against each of dengue serotypes 1, 2, 3 and 4.

37. Method according to any one of claims 34 to 36, wherein in the method of (b) antibodies against one dengue serotype are depleted using beads coupled to virions of said dengue serotype.

38. Method according to any one of claims 34 to 37, wherein in the method of (b) the level of neutralizing antibodies in the depleted sample is determined using reporter virus particles.

39. Method according to any one of claims 34 to 38, wherein the method of (b) comprises the steps of:
(i) providing beads coupled to virions of a dengue serotype;
(ii) incubating the beads of (i) with a serum sample;
(iii) separating the beads from the serum sample, thereby providing a depleted serum sample;
(iv) mixing the depleted serum sample with reporter virus particles and incubating the resulting mixture under conditions suitable for virus neutralization, thereby providing a neutralized sample;
(v) incubating the neutralized sample with permissive cells for a suitable period; and
(vi) detecting expression of the reporter protein, and quantifying it for calculation of neutralizing antibody titer.

40. Method according to any one of claims 37 to 39, wherein the beads are magnetic beads.

41. Method according to any one of claims 37 to 40, wherein the virions of said dengue serotype are virions of dengue serotype 2.

42. Method according to any one of claims 38 to 41, wherein upon infection of permissive cells the reporter virus particles express luciferase.

43. Method according to claim 42, wherein the permissive cells are Raj i-DC-SIGN cells.

44. Method according to any one of the preceding claims, wherein the method of (c) comprises an ELISA assay.

45. Method according to claim 44, wherein the ELISA assay is an indirect ELISA assay.

46. Method according to claim 44 or 45, wherein the ELISA assay comprises the following steps:
(i) providing a microplate coated with the non-structural protein or an antigenic fragment thereof;
(ii) adding diluted serum samples to the coated microplate under conditions such that antibodies present in the serum samples can bind to the non-structural protein or an antigenic fragment thereof;
(iii) washing the microplate to remove unbound antibodies;
(iv) adding an enzyme-conjugated antibody capable of binding to the antibodies present in the serum samples under conditions such that the enzyme-conjugated antibody can bind to the antibodies present in the serum samples;
(v) washing the microplate to remove unbound antibodies;
(vi) adding the enzyme substrate under suitable conditions such that a color signal is produced; and
(vii) detecting and quantifying the color signal.

47. Method according to any one of claims 44 to 46, wherein the non-structural protein is NS1.

48. Method according to claim 46 or 47, wherein the enzyme-conjugated antibody is an anti-IgG antibody.

49. Method according to any one of the preceding claims, wherein the method of (d) comprises an ELISA assay.

50. Method according to claim 49, wherein the ELISA assay is a sandwich ELISA assay.

51. Method according to claim 49 or 50, wherein the ELISA assay comprises the following steps:
(x) providing a microplate coated with a monoclonal antibody capable of binding to all dengue serotypes;
(xi) adding a live virion of a dengue serotype selected from the group consisting of serotypes 1, 2, 3 or 4 to the coated microplate;
(xii) washing the microplate to remove unbound live virions;
(xiii) adding diluted serum samples to the microplate under conditions such that antibodies present in the serum samples can bind to the live virion;
(xiv) washing the microplate to remove unbound antibodies;
(xv) adding an enzyme-conjugated antibody capable of binding to the antibodies present in the serum samples under conditions such that the enzyme-conjugated antibody can bind to the antibodies present in the serum samples;
(xvi) washing the microplate to remove unbound antibodies;
(xvii) adding the enzyme substrate under suitable conditions such that a color signal is produced; and
(xviii) detecting and quantifying the color signal.

52. Method according to claim 51, wherein the monoclonal antibody capable of binding to all dengue serotypes binds to a conserved epitope in the E protein.

53. Method according to claim 51 or 52, wherein the live virion is selected from the group consisting of:
(1) a chimeric dengue serotype 2/1 strain,
(2) a dengue serotype 2 strain,
(3) a chimeric dengue serotype 2/3 strain, and
(4) a chimeric dengue serotype 2/4 strain.

54. Method according to any one of claims 51 to 53, wherein the enzyme-conjugated antibody is an anti-IgG antibody.

55. Method according to any one of claims 34 to 54, wherein the method of (e)
Step 1: contacting an amount of a microsphere complex comprising a microsphere coupled to a flavivirus antigen with the sample to allow binding of the flavivirus-reactive complement-fixing antibodies in the sample to the flavivirus antigen;
Step 2: contacting an amount of complement component 1q (C1q) with the complement-fixing antibodies bound to the flavivirus antigen in step 1 to allow binding of the C1q to the heavy chain constant region of the complement-fixing antibodies;
Step 3: contacting an amount of a reporter antibody with the C1q bound to the complement-fixing antibodies in step 2 to allow binding of the reporter antibody to the C1q, wherein the reporter antibody binds to the C1q with the variable region of the reporter antibody and wherein the reporter antibody is attached to a detectable label; and
Step 4: detecting a signal from the reporter antibody bound to the C1q in step 3, wherein the signal is indicative for the presence and/or amount of the reporter antibody and wherein the presence and/or amount of the reporter antibody is indicative for the presence and/or amount of flavivirus-reactive complement-fixing antibodies in the sample.

56. Method according to any one of claims 34 to 55, wherein the tetravalent dengue virus composition comprises four live, attenuated dengue virus strains.

57. Method according to claim 56, wherein the four live, attenuated dengue virus strains are:
(v) a chimeric dengue serotype 2/1 strain,
(vi) a dengue serotype 2 strain,
(vii) a chimeric dengue serotype 2/3 strain, and
(viii) a chimeric dengue serotype 2/4 strain.

58. Method according to claim 56 or 57, wherein each one of the four live, attenuated dengue virus strains has attenuating mutations in the 5′-noncoding region (NCR) at nucleotide 57 from cytosine to thymine, in the NS1 gene at nucleotide 2579 from guanine to adenine resulting in an amino acid change at position 828 of the NS1 protein from glycine to asparagine, and in the NS3 gene at nucleotide 5270 from adenine to thymine resulting in an amino acid change at position 1725 of the NS3 protein from glutamine to valine.

59. Method according to any one of claims 34 to 58, wherein a first serum sample is obtained before the tetravalent dengue virus composition is administered to the subject and at least one second serum sample is obtained after the tetravalent dengue virus composition has been administered to the subject.

60. Method according to claim 59, wherein the at least one second serum sample is obtained 30 to 360 days after the tetravalent dengue virus composition has been administered to the subject.

61. A method for characterizing the immune response of a subject to a virus-containing vaccine composition administered to said subject, comprising performing with a serum sample from said subject at least two methods selected from the group consisting of:
(a) a method to determine the level of neutralizing antibodies in said sample;
(b) a method comprising depleting antibodies against one dengue serotype from said sample followed by determining in the depleted sample the level of neutralizing antibodies against at least one serotype different from the dengue serotype used for depleting the antibodies, to detect the presence of type-specific and cross-reactive neutralizing antibodies in said sample;
(c) a method to determine the level of antibodies against non-structural protein 1 of dengue virus in said sample;
(d) a method to determine the level of dengue-binding antibodies in said sample;
(e) a method to determine the presence and/or amount of flavivirus-reactive complement-fixing antibodies in said sample; and
(f) the avidity assay method to determine avidity or binding antibody response according to any of claims 1 to 27.

62. The method for characterizing the immune response of a subject to a virus-containing vaccine composition according to claim 61, wherein the method of (a) is as defined in claim 35 or 36.

63. The method for characterizing the immune response of a subject to a virus-containing vaccine composition according to claim 61 or 62, wherein the method of (b) is as defined in any one of claims 37 to 43.

64. The method for characterizing the immune response of a subject to a virus-containing vaccine composition according to any one of claims 61 to 63, wherein the method of (c) is as defined in any one of claims 44 to 48.

65. The method for characterizing the immune response of a subject to a virus-containing vaccine composition according to any one of claims 61 to 64, wherein the method of (d) is as defined in any one of claims 49 to 54.

66. The method for characterizing the immune response of a subject to a virus-containing vaccine composition according to any one of claims 61 to 65, wherein the method of (e) is as defined in any one of claims 55 to 60.

67. A method for predicting the protective efficacy of a dengue vaccine candidate comprising
determining the presence and/or amount of at least two immune response parameters selected from the group consisting of neutralizing antibodies, serotype specific antibodies, cross-reactive neutralizing antibodies, complement-fixing antibodies, dengue total binding antibodies, high affinity antibodies against dengue virus and antibodies against dengue non-structural protein 1 in a blood sample from a subject vaccinated with the dengue vaccine candidate, and
predicting the dengue vaccine candidate to provide protective efficacy if the presence of at least two immune response parameters selected from the group consisting of neutralizing antibodies, serotype specific and/or cross-reactive neutralizing antibodies, cross-reactive neutralizing antibodies, complement-fixing antibodies, dengue total binding antibodies, high affinity antibodies against dengue virus and antibodies against dengue non-structural protein 1 (NS1) is determined in the blood sample.

68. The method for predicting the protective efficacy according to claim 67, wherein the at least two immune response parameters are selected from the group consisting of
(i) serotype-specific neutralizing antibodies against at least one dengue structural protein,
(ii) cross-reactive neutralizing antibodies against at least one dengue structural protein,
(iii) cross-reactive antibodies against at least one dengue non-structural protein,
(iv) complement-fixing antibodies against at least one dengue structural protein,
(v) dengue total binding IgG response, and
(vi) high affinity antibodies against dengue antigens,
preferably the at least two immune response parameters are selected from the group consisting of
(i) neutralizing antibodies against at least one dengue serotype
(ii) serotype-specific neutralizing antibodies against at least one dengue serotype,
(ii) cross-reactive neutralizing antibodies against at least one dengue serotype,
(iii) antibodies against dengue non-structural protein 1 of at least one dengue serotype,
(iv) complement-fixing antibodies against at least one dengue serotype,
(v) dengue total binding IgG response against at least one dengue serotype, and
(vi) high affinity antibodies against dengue antigens from at least one dengue serotype.

69. The method for predicting the protective efficacy according to claim 68, wherein the dengue structural protein is dengue E protein and/or the dengue non-structural protein is dengue NS1 protein.

70. The method for predicting the protective efficacy according to any one of claims 67 to 69, wherein the immune response parameters are determined by any of the methods for characterizing an immune response according to any one of claims 61 to 66.

71. A method for preparing a vaccine formulation comprising
performing the method for predicting the protective efficacy of a dengue vaccine candidate according to any one of claims 67 to 70; and
formulating the vaccine candidate predicted to provide protective efficacy with a pharmaceutically acceptable excipient.

72. Vaccine formulation obtainable by the method of claim 71.


SEQUENCE LISTING (8 sequences)

SEQ ID NO: 1 - DNA, 10723 bp, chimeric dengue seroytpe 2/1 (MVS)
agttgttagtctacgtggaccgacaaagacagattctttgagggagctaagctcaatgtagttctaacagttttttaattagagagcagatctctgatgaataaccaacggaaaaaggcgaaaaacacgcctttcaatatgctgaaacgcgagagaaaccgcgtgtcgactgtgcaacagctgacaaagagattctcacttggaatgctgcagggacgaggaccattaaaactgttcatggccctggtggcgttccttcgtttcctaacaatcccaccaacagcagggatattgaagagatggggaacaattaaaaaatcaaaagctattaatgttttgagagggttcaggaaagagattggaaggatgctgaacatcttgaataggagacgcagatctgcaggcatgatcattatgctgattccaacagtgatggcgttccatttaaccacgcgtgggggagagccgcatatgatagttagcaagcaggaaagaggaaagtcacttttgttcaagacctctgcaggtgtcaacatgtgcaccctcattgcgatggatttgggagagttgtgtgaggacacgatgacctacaaatgcccccggatcactgaggcggaaccagatgacgttgactgttggtgcaatgccacggacacatgggtgacctatggaacgtgctctcaaactggcgaacaccgacgagacaaacgttccgtcgcattggccccacacgtggggcttggcctagaaacaagagccgaaacgtggatgtcctctgaaggtgcttggaaacagatacaaaaagtagagacttgggctctgagacatccaggattcacggtgatagccctttttctagcacatgccataggaacatccatcacccagaaagggatcattttcattttgctgatgctggtaacaccatctatggccatgcgatgcgtgggaataggcaacagagacttcgtggaaggactgtcaggagcaacatgggtggatgtggtactggagcatggaagttgcgtcaccaccatggcaaaaaacaaaccaacactggacattgaactcttgaagacggaggtcacaaaccctgcagttctgcgtaaattgtgcattgaagctaaaatatcaaacaccaccaccgattcgagatgtccaacacaaggagaagccacactggtggaagaacaagacgcgaactttgtgtgccgacgaacgttcgtggacagaggctggggcaatggctgtgggctattcggaaaaggtagtctaataacgtgtgccaagtttaagtgtgtgacaaaactagaaggaaagatagttcaatatgaaaacctaaaatattcagtgatagtcaccgtccacactggagatcagcaccaggtgggaaatgagactacagaacatggaacaactgcaaccataacacctcaagctcctacgtcggaaatacagctgaccgactacggaacccttacattagattgttcacctaggacagggctagattttaacgagatggtgttgctgacaatgaaagaaagatcatggcttgtccacaaacaatggttcctagacttaccactgccttggacctctggggcttcaacatcccaagagacttggaacagacaagatttactggtcacatttaagacagctcatgcaaagaagcaggaagtagtcgtactaggatcacaagaaggagcaatgcacactgcgctgactggagcgacagaaatccaaacgtcaggaacgacaacaattttcgcaggacacctaaaatgcagactaaaaatggacaaactaactttaaaagggatgtcatatgtgatgtgcacaggctcattcaagttagagaaagaagtggctgagacccagcatggaactgttctggtgcaggttaaatatgaaggaacagacgcaccatgcaagattcccttttcgacccaagatgagaaaggagcaacccagaatgggagattaataacagccaaccccatagtcactgacaaagaaaaaccagtcaatattgaggcagaaccaccctttggtgagagctacatcgtggtaggagcaggtgaaaaagctttgaaactaagctggttcaagaaaggaagcagcatagggaaaatgtttgaagcaactgcccgaggagcacgaaggatggccattctgggagacaccgcatgggacttcggttctataggaggagtgttcacgtctatgggaaaactggtacaccaggtttttggaactgcatatggagttttgtttagcggagtttcttggaccatgaaaataggaatagggattctgctgacatggctaggattaaattcaaggaacacgtccctttcgatgatgtgcatcgcagccggcattgtgacactgtatttgggagtcatggtgcaggccgatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagcccatgaagaggacatttgtggaatccgctcagtaacaagactggagaatctgatgtggaaacaaataacaccagaattgaatcacattctatcagaaaatgaggtgaagttaactattatgacaggagacatcaaaggaatcatgcaggcaggaaaacgatctctgcggcctcagcccactgagctgaagtattcatggaaaacatggggcaaagcaaaaatgctctctacagagtctcataaccagacctttctcattgatggccccgaaacagcagaatgccccaacacaaatagagcttggaattcgttggaagttgaagactatggctttggagtattcaccaccaatatatggctaaaattgaaagaaaaacaggatgtattctgcgactcaaaactcatgtcagcggccataaaagacaacagagccgtccatgccgatatgggttattggatagaaagtgcactcaatgacacatggaagatagagaaagcctctttcattgaagttaaaaactgccactggccaaaatcacacaccctctggagcaatggagtgctagaaagtgagatgataattccaaagaatctcgctggaccagtgtctcaacacaactatagaccaggctaccatacacaaataacaggaccatggcatctaggtaagcttgagatggactttgatttctgtgatggaacaacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaggagaaagaagagaatttggtcaactccttggtcacagctggacatgggcaggtcgacaacttttcactaggagtcttgggaatggcattgttcctggaggaaatgcttaggacccgagtaggaacgaaacatgcaatactactagttgcagtttcttttgtgacattgatcacagggaacatgtcctttagagacctgggaagagtgatggttatggtaggcgccactatgacggatgacataggtatgggcgtgacttatcttgccctactagcagccttcaaagtcagaccaacttttgcagctggactactcttgagaaagctgacctccaaggaattgatgatgactactataggaattgtactcctctcccagagcaccctaccagagaccattcttgagttgactgatgcgttagccttaggcatgatggtcctcaaaatggtgagaaatatggaaaagtatcaattggcagtgactatcatggctatcttgtgcgtcccaaacgcagtgatattacaaaacgcatggaaagtgagttgcacaatattggcagtggtgtccgtttccccactgttcttaacatcctcacagcaaaaaacagattggataccattagcattgacgatcaaaggtctcaatccaacagctatttttctaacaaccctctcaagaaccagcaagaaaaggagctggccattaaatgaggctatcatggcagtcgggatggtgagcattttagccagttctctcctaaaaaatgatattcccatgacaggaccattagtggctggagggctcctcactgtgtgctacgtgctcactggacgatcggccgatttggaactggagagagcagccgatgtcaaatgggaagaccaggcagagatatcaggaagcagtccaatcctgtcaataacaatatcagaagatggtagcatgtcgataaaaaatgaagaggaagatcaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaattaagcaaaaagggattcttggatattcccagatcggagccggagtttacaaagaaggaacattccatacaatgtggcatgtcacacgtggcgctgttctaatgcataaaggaaagaggattgaaccatcatgggcggacgtcaagaaagacctaatatcatatggaggaggctggaagttagaaggagaatggaaggaaggagaagaagtccaggtattggcactggagcctggaaaaaatccaagagccgtccaaacgaaacctggtcttttcaaaaccaacgccggaacaataggtgctgtatctctggacttttctcctggaacgtcaggatctccaattatcgacaaaaaaggaaaagttgtgggtctttatggtaatggtgttgttacaaggagtggagcatatgtgagtgctatagcccagactgaaaaaagcattgaagacaacccagagatcgaagatgacattttccgaaagagaagactgaccatcatggacctccacccaggagcgggaaagacgaagagataccttccggccatagtcagagaagctataaaacggggtttgagaacattaatcttggcccccactagagttgtggcagctgaaatggaggaagcccttagaggacttccaataagataccagaccccagccatcagagctgtgcacaccgggcgggagattgtggacctaatgtgtcatgccacatttaccatgaggctgctatcaccagttagagtgccaaactacaacctgattatcatggacgaagcccatttcacagacccagcaagtatagcagctagaggatacatctcaactcgagtggagatgggtgaggcagctgggatttttatgacagccactcccccgggaagcagagacccatttcctcagagcaatgcaccaatcatagatgaagaaagagaaatccctgaacgctcgtggaattccggacatgaatgggtcacggattttaaagggaagactgtttggttcgttccaagtataaaagcaggaaatgatatagcagcttgcctgaggaaaaatggaaagaaagtgatacaactcagtaggaagacctttgattctgagtatgtcaagactagaaccaatgattgggacttcgtggttacaactgacatttcagaaatgggtgccaatttcaaggctgagagggttatagaccccagacgctgcatgaaaccagtcatactaacagatggtgaagagcgggtgattctggcaggacctatgccagtgacccactctagtgcagcacaaagaagagggagaataggaagaaatccaaaaaatgagaatgaccagtacatatacatgggggaacctctggaaaatgatgaagactgtgcacactggaaagaagctaaaatgctcctagataacatcaacacgccagaaggaatcattcctagcatgttcgaaccagagcgtgaaaaggtggatgccattgatggcgaataccgcttgagaggagaagcaaggaaaacctttgtagacttaatgagaagaggagacctaccagtctggttggcctacagagtggcagctgaaggcatcaactacgcagacagaaggtggtgttttgatggagtcaagaacaaccaaatcctagaagaaaacgtggaagttgaaatctggacaaaagaaggggaaaggaagaaattgaaacccagatggttggatgctaggatctattctgacccactggcgctaaaagaatttaaggaatttgcagccggaagaaagtctctgaccctgaacctaatcacagaaatgggtaggctcccaaccttcatgactcagaaggcaagagacgcactggacaacttagcagtgctgcacacggctgaggcaggtggaagggcgtacaaccatgctctcagtgaactgccggagaccctggagacattgcttttactgacacttctggctacagtcacgggagggatctttttattcttgatgagcgcaaggggcatagggaagatgaccctgggaatgtgctgcataatcacggctagcatcctcctatggtacgcacaaatacagccacactggatagcagcttcaataatactggagttttttctcatagttttgcttattccagaacctgaaaaacagagaacaccccaagacaaccaactgacctacgttgtcatagccatcctcacagtggtggccgcaaccatggcaaacgagatgggtttcctagaaaaaacgaagaaagatctcggattgggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtgtccctaacagctatagccaaccaagccacagtgttaatgggtctcgggaaaggatggccattgtcaaagatggacatcggagttccccttctcgccattggatgctactcacaagtcaaccccataactctcacagcagctcttttcttattggtagcacattatgccatcatagggccaggactccaagcaaaagcaaccagagaagctcagaaaagagcagcggcgggcatcatgaaaaacccaactgtcgatggaataacagtgattgacctagatccaataccttatgatccgaagtttgaaaagcagttgggacaagtaatgctcctagtcctctgcgtgactcaagtattgatgatgaggactacatgggctctgtgtgaggctttaaccttagctaccgggcccatctccacattgtgggaaggaaatccagggaggttttggaacactaccattgcggtgtcaatggctaacatttttagagggagttacttggccggagctggacttctcttttctattatgaagaacacaaccaacacaagaaggggaactggcaacataggagagacgcttggagagaaatggaaaagccgattgaacgcattgggaaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgttgagagaaacatggtcacaccagaagggaaagtagtggacctcggttgtggcagaggaggctggtcatactattgtggaggactaaagaatgtaagagaagtcaaaggcctaacaaaaggaggaccaggacacgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtagaaaattggttgaacaacaacactcaattttgcataaaggttctcaacccatatatgccctcagtcatagaaaaaatggaagcactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtggtcaggctgctgacaaaaccttgggacgtcgtccccatggtgacacagatggcaatgacagacacgactccatttggacaacagcgcgtttttaaagagaaagtggacacgagaacccaagaaccgaaagaaggcacgaagaaactaatgaaaataacagcagagtggctttggaaagaattagggaagaaaaagacacccaggatgtgcaccagagaagaattcacaagaaaggtgagaagcaatgcagccttgggggccatattcactgatgagaacaagtggaagtcggcacgtgaggctgttgaagatagtaggttttgggagctggttgacaaggaaaggaatctccatcttgaaggaaagtgtgaaacatgtgtgtacaacatgatgggaaaaagagagaagaagctaggggaattcggcaaggcaaaaggcagcagagccatatggtacatgtggcttggagcacgcttcttagagtttgaagccctaggattcttaaatgaagatcactggttctccagagagaactccctgagtggagtggaaggagaagggctgcacaagctaggttacattctaagagacgtgagcaagaaagagggaggagcaatgtatgccgatgacaccgcaggatgggatacaagaatcacactagaagacctaaaaaatgaagaaatggtaacaaaccacatggaaggagaacacaagaaactagccgaggccattttcaaactaacgtaccaaaacaaggtggtgcgtgtgcaaagaccaacaccaagaggcacagtaatggacatcatatcgagaagagaccaaagaggtagtggacaagttggcacctatggactcaatactttcaccaatatggaagcccaactaatcagacagatggagggagaaggagtctttaaaagcattcagcacctaacaatcacagaagaaatcgctgtgcaaaactggttagcaagagtggggcgcgaaaggttatcaagaatggccatcagtggagatgattgtgttgtgaaacctttagatgacaggttcgcaagcgctttaacagctctaaatgacatgggaaagattaggaaagacatacaacaatgggaaccttcaagaggatggaatgattggacacaagtgcccttctgttcacaccatttccatgagttaatcatgaaagacggtcgcgtactcgttgttccatgtagaaaccaagatgaactgattggcagagcccgaatctcccaaggagcagggtggtctttgcgggagacggcctgtttggggaagtcttacgcccaaatgtggagcttgatgtacttccacagacgcgacctcaggctggcggcaaatgctatttgctcggcagtaccatcacattgggttccaacaagtcgaacaacctggtccatacatgctaaacatgaatggatgacaacggaagacatgctgacagtctggaacagggtgtggattcaagaaaacccatggatggaagacaaaactccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaagaatacacagattacatgccatccatgaaaagattcagaagagaagaggaagaagcaggagttctgtggtagaaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 2 - protein, 3391 aa, chimeric dengue serotype 2/1 (MVS)
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRRSAGMIIMLIPTVMAFHLTTRGGEPHMIVSKQERGKSLLFKTSAGVNMCTLIAMDLGELCEDTMTYKCPRITEAEPDDVDCWCNATDTWVTYGTCSQTGEHRRDKRSVALAPHVGLGLETRAETWMSSEGAWKQIQKVETWALRHPGFTVIALFLAHAIGTSITQKGIIFILLMLVTPSMAMRCVGIGNRDFVEGLSGATWVDVVLEHGSCVTTMAKNKPTLDIELLKTEVTNPAVLRKLCIEAKISNTTTDSRCPTQGEATLVEEQDANFVCRRTFVDRGWGNGCGLFGKGSLITCAKFKCVTKLEGKIVQYENLKYSVIVTVHTGDQHQVGNETTEHGTTATITPQAPTSEIQLTDYGTLTLDCSPRTGLDFNEMVLLTMKERSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSTQDEKGATQNGRLITANPIVTDKEKPVNIEAEPPFGESYIVVGAGEKALKLSWFKKGSSIGKMFEATARGARRMAILGDTAWDFGSIGGVFTSMGKLVHQVFGTAYGVLFSGVSWTMKIGIGILLTWLGLNSRNTSLSMMCIAAGIVTLYLGVMVQADSGCVVSWKNKELKCGSGIFITDNVHTWTEQYKFQPESPSKLASAIQKAHEEDICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTLPETILELTDALALGMMVLKMVRNMEKYQLAVTIMAILCVPNAVILQNAWKVSCTILAVVSVSPLFLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEDQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAVHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKARDALDNLAVLHTAEAGGRAYNHALSELPETLETLLLLTLLATVTGGIFLFLMSARGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 3 - DNA, 10723 bp, dengue serotype 2 (MVS)
agttgttagtctacgtggaccgacaaagacagattctttgagggagctaagctcaatgtagttctaacagttttttaattagagagcagatctctgatgaataaccaacggaaaaaggcgaaaaacacgcctttcaatatgctgaaacgcgagagaaaccgcgtgtcgactgtgcaacagctgacaaagagattctcacttggaatgctgcagggacgaggaccattaaaactgttcatggccctggtggcgttccttcgtttcctaacaatcccaccaacagcagggatattgaagagatggggaacaattaaaaaatcaaaagctattaatgttttgagagggttcaggaaagagattggaaggatgctgaacatcttgaataggagacgcagatctgcaggcatgatcattatgctgattccaacagtgatggcgttccatttaaccacacgtaacggagaaccacacatgatcgtcagcagacaagagaaagggaaaagtcttctgtttaaaacagaggttggcgtgaacatgtgtaccctcatggccatggaccttggtgaattgtgtgaagacacaatcacgtacgagtgtccccttctcaggcagaatgagccagaagacatagactgttggtgcaactctacgtccacgtgggtaacttatgggacgtgtaccaccatgggagaacatagaagagaaaaaagatcagtggcactcgttccacatgtgggaatgggactggagacacgaactgaaacatggatgtcatcagaaggggcctggaaacatgtccagagaattgaaacttggatcttgagacatccaggcttcaccatgatggcagcaatcctggcatacaccataggaacgacacatttccaaagagccctgatcttcatcttactgacagctgtcactccttcaatgacaatgcgttgcataggaatgtcaaatagagactttgtggaaggggtttcaggaggaagctgggttgacatagtcttagaacatggaagctgtgtgacgacgatggcaaaaaacaaaccaacattggattttgaactgataaaaacagaagccaaacagcctgccaccctaaggaagtactgtatagaggcaaagctaaccaacacaacaacagaatctcgctgcccaacacaaggggaacccagcctaaatgaagagcaggacaaaaggttcgtctgcaaacactccatggtagacagaggatggggaaatggatgtggactatttggaaagggaggcattgtgacctgtgctatgttcagatgcaaaaagaacatggaaggaaaagttgtgcaaccagaaaacttggaatacaccattgtgataacacctcactcaggggaagagcatgcagtcggaaatgacacaggaaaacatggcaaggaaatcaaaataacaccacagagttccatcacagaagcagaattgacaggttatggcactgtcacaatggagtgctctccaagaacgggcctcgacttcaatgagatggtgttgctgcagatggaaaataaagcttggctggtgcacaggcaatggttcctagacctgccgttaccatggttgcccggagcggacacacaagggtcaaattggatacagaaagagacattggtcactttcaaaaatccccatgcgaagaaacaggatgttgttgttttaggatcccaagaaggggccatgcacacagcacttacaggggccacagaaatccaaatgtcatcaggaaacttactcttcacaggacatctcaagtgcaggctgagaatggacaagctacagctcaaaggaatgtcatactctatgtgcacaggaaagtttaaagttgtgaaggaaatagcagaaacacaacatggaacaatagttatcagagtgcaatatgaaggggacggctctccatgcaagatcccttttgagataatggatttggaaaaaagacatgtcttaggtcgcctgattacagtcaacccaattgtgacagaaaaagatagcccagtcaacatagaagcagaacctccatttggagacagctacatcatcataggagtagagccgggacaactgaagctcaactggtttaagaaaggaagttctatcggccaaatgtttgagacaacaatgaggggggcgaagagaatggccattttaggtgacacagcctgggattttggatccttgggaggagtgtttacatctataggaaaggctctccaccaagtctttggagcaatctatggagctgccttcagtggggtttcatggactatgaaaatcctcataggagtcattatcacatggataggaatgaattcacgcagcacctcactgtctgtgacactagtattggtgggaattgtgacactgtatttgggagtcatggtgcaggccgatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagcccatgaagaggacatttgtggaatccgctcagtaacaagactggagaatctgatgtggaaacaaataacaccagaattgaatcacattctatcagaaaatgaggtgaagttaactattatgacaggagacatcaaaggaatcatgcaggcaggaaaacgatctctgcggcctcagcccactgagctgaagtattcatggaaaacatggggcaaagcaaaaatgctctctacagagtctcataaccagacctttctcattgatggccccgaaacagcagaatgccccaacacaaatagagcttggaattcgttggaagttgaagactatggctttggagtattcaccaccaatatatggctaaaattgaaagaaaaacaggatgtattctgcgactcaaaactcatgtcagcggccataaaagacaacagagccgtccatgccgatatgggttattggatagaaagtgcactcaatgacacatggaagatagagaaagcctctttcattgaagttaaaaactgccactggccaaaatcacacaccctctggagcaatggagtgctagaaagtgagatgataattccaaagaatctcgctggaccagtgtctcaacacaactatagaccaggctaccatacacaaataacaggaccatggcatctaggtaagcttgagatggactttgatttctgtgatggaacaacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaggagaaagaagagaatttggtcaactccttggtcacagctggacatgggcaggtcgacaacttttcactaggagtcttgggaatggcattgttcctggaggaaatgcttaggacccgagtaggaacgaaacatgcaatactactagttgcagtttcttttgtgacattgatcacagggaacatgtcctttagagacctgggaagagtgatggttatggtaggcgccactatgacggatgacataggtatgggcgtgacttatcttgccctactagcagccttcaaagtcagaccaacttttgcagctggactactcttgagaaagctgacctccaaggaattgatgatgactactataggaattgtactcctctcccagagcaccataccagagaccattcttgagttgactgatgcgttagccttaggcatgatggtcctcaaaatggtgagaaatatggaaaagtatcaattggcagtgactatcatggctatcttgtgcgtcccaaacgcagtgatattacaaaacgcatggaaagtgagttgcacaatattggcagtggtgtccgtttccccactgttcttaacatcctcacagcaaaaaacagattggataccattagcattgacgatcaaaggtctcaatccaacagctatttttctaacaaccctctcaagaaccagcaagaaaaggagctggccattaaatgaggctatcatggcagtcgggatggtgagcattttagccagttctctcctaaaaaatgatattcccatgacaggaccattagtggctggagggctcctcactgtgtgctacgtgctcactggacgatcggccgatttggaactggagagagcagccgatgtcaaatgggaagaccaggcagagatatcaggaagcagtccaatcctgtcaataacaatatcagaagatggtagcatgtcgataaaaaatgaagaggaagaacaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaattaagcaaaaagggattcttggatattcccagatcggagccggagtttacaaagaaggaacattccatacaatgtggcatgtcacacgtggcgctgttctaatgcataaaggaaagaggattgaaccatcatgggcggacgtcaagaaagacctaatatcatatggaggaggctggaagttagaaggagaatggaaggaaggagaagaagtccaggtattggcactggagcctggaaaaaatccaagagccgtccaaacgaaacctggtcttttcaaaaccaacgccggaacaataggtgctgtatctctggacttttctcctggaacgtcaggatctccaattatcgacaaaaaaggaaaagttgtgggtctttatggtaatggtgttgttacaaggagtggagcatatgtgagtgctatagcccagactgaaaaaagcattgaagacaacccagagatcgaagatgacattttccgaaagagaagactgaccatcatggacctccacccaggagcgggaaagacgaagagataccttccggccatagtcagagaagctataaaacggggtttgagaacattaatcttggcccccactagagttgtggcagctgaaatggaggaagcccttagaggacttccaataagataccagaccccagccatcagagctgtgcacaccgggcgggagattgtggacctaatgtgtcatgccacatttaccatgaggctgctatcaccagttagagtgccaaactacaacctgattatcatggacgaagcccatttcacagacccagcaagtatagcagctagaggatacatctcaactcgagtggagatgggtgaggcagctgggatttttatgacagccactcccccgggaagcagagacccatttcctcagagcaatgcaccaatcatagatgaagaaagagaaatccctgaacgctcgtggaattccggacatgaatgggtcacggattttaaagggaagactgtttggttcgttccaagtataaaagcaggaaatgatatagcagcttgcctgaggaaaaatggaaagaaagtgatacaactcagtaggaagacctttgattctgagtatgtcaagactagaaccaatgattgggacttcgtggttacaactgacatttcagaaatgggtgccaatttcaaggctgagagggttatagaccccagacgctgcatgaaaccagtcatactaacagatggtgaagagcgggtgattctggcaggacctatgccagtgacccactctagtgcagcacaaagaagagggagaataggaagaaatccaaaaaatgagaatgaccagtacatatacatgggggaacctctggaaaatgatgaagactgtgcacactggaaagaagctaaaatgctcctagataacatcaacacgccagaaggaatcattcctagcatgttcgaaccagagcgtgaaaaggtggatgccattgatggcgaataccgcttgagaggagaagcaaggaaaacctttgtagacttaatgagaagaggagacctaccagtctggttggcctacagagtggcagctgaaggcatcaactacgcagacagaaggtggtgttttgatggagtcaagaacaaccaaatcctagaagaaaacgtggaagttgaaatctggacaaaagaaggggaaaggaagaaattgaaacccagatggttggatgctaggatctattctgacccactggcgctaaaagaatttaaggaatttgcagccggaagaaagtctctgaccctgaacctaatcacagaaatgggtaggctcccaaccttcatgactcagaaggcaagagacgcactggacaacttagcagtgctgcacacggctgaggcaggtggaagggcgtacaaccatgctctcagtgaactgccggagaccctggagacattgcttttactgacacttctggctacagtcacgggagggatctttttattcttgatgagcgcaaggggcatagggaagatgaccctgggaatgtgctgcataatcacggctagcatcctcctatggtacgcacaaatacagccacactggatagcagcttcaataatactggagttttttctcatagttttgcttattccagaacctgaaaaacagagaacaccccaagacaaccaactgacctacgttgtcatagccatcctcacagtggtggccgcaaccatggcaaacgagatgggtttcctagaaaaaacgaagaaagatctcggattgggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtgtccctaacagctatagccaaccaagccacagtgttaatgggtctcgggaaaggatggccattgtcaaagatggacatcggagttccccttctcgccattggatgctactcacaagtcaaccccataactctcacagcagctcttttcttattggtagcacattatgccatcatagggccaggactccaagcaaaagcaaccagagaagctcagaaaagagcagcggcgggcatcatgaaaaacccaactgtcgatggaataacagtgattgacctagatccaataccttatgatccaaagtttgaaaagcagttgggacaagtaatgctcctagtcctctgcgtgactcaagtattgatgatgaggactacatgggctctgtgtgaggctttaaccttagctaccgggcccatctccacattgtgggaaggaaatccagggaggttttggaacactaccattgcggtgtcaatggctaacatttttagagggagttacttggccggagctggacttctcttttctattatgaagaacacaaccaacacaagaaggggaactggcaacataggagagacgcttggagagaaatggaaaagccgattgaacgcattgggaaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgttgagagaaacatggtcacaccagaagggaaagtagtggacctcggttgtggcagaggaggctggtcatactattgtggaggactaaagaatgtaagagaagtcaaaggcctaacaaaaggaggaccaggacacgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtagaaaattggttgaacaacaacactcaattttgcataaaggttctcaacccatatatgccctcagtcatagaaaaaatggaagcactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtggtcaggctgctgacaaaaccttgggacgtcgtccccatggtgacacagatggcaatgacagacacgactccatttggacaacagcgcgtttttaaagagaaagtggacacgagaacccaagaaccgaaagaaggcacgaagaaactaatgaaaataacagcagagtggctttggaaagaattagggaagaaaaagacacccaggatgtgcaccagagaagaattcacaagaaaggtgagaagcaatgcagccttgggggccgtattcactgatgagaacaagtggaagtcggcacgtgaggctgttgaagatagtaggttttgggagctggttgacaaggaaaggaatctccatcttgaaggaaagtgtgaaacatgtgtgtacaacatgatgggaaaaagagagaagaagctaggggaattcggcaaggcaaaaggcagcagagccatatggtacatgtggcttggagcacgcttcttagagtttgaagccctaggattcttaaatgaagatcactggttctccagagagaactccctgagtggagtggaaggagaagggctgcacaagctaggttacattctaagagacgtgagcaagaaagagggaggagcaatgtatgccgatgacaccgcaggatgggatacaagaatcacactagaagacctaaaaaatgaagaaatggtaacaaaccacatggaaggagaacacaagaaactagccgaggccattttcaaactaacgtaccaaaacaaggtggtgcgtgtgcaaagaccaacaccaagaggcacagtaatggacatcatatcgagaagagaccaaagaggtagtggacaagttggcacctatggactcaatactttcaccaatatggaagcccaactaatcagacagatggagggagaaggagtctttaaaagcattcagcacctaacaatcacagaagaaatcgctgtgcaaaactggttagcaagagtggggcgcgaaaggttatcaagaatggccatcagtggagatgattgtgttgtgaaacctttagatgacaggttcgcaagcgctttaacagctctaaatgacatgggaaagattaggaaagacatacaacaatgggaaccttcaagaggatggaatgattggacacaagtgcccttctgttcacaccatttccatgagttaatcatgaaagacggtcgcgtactcgttgttccatgtagaaaccaagatgaactgattggcagagcccgaatctcccaaggagcagggtggtctttgcgggagacggcctgtttggggaagtcttacgcccaaatgtggagcttgatgtacttccacagacgcgacctcaggctggcggcaaatgctatttgctcggcagtaccatcacattgggttccaacaagtcgaacaacctggtccatacatgctaaacatgaatggatgacaacggaagacatgctgacagtctggaacagggtgtggattcaagaaaacccatggatggaagacaaaactccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaagaatacacagattacatgccatccatgaaaagattcagaagagaagaggaagaagcaggagttctgtggtagaaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 4 - protein, 3391 aa, dengue seroytpe 2 (MVS)
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRRSAGMIIMLIPTVMAFHLTTRNGEPHMIVSRQEKGKSLLFKTEVGVNMCTLMAMDLGELCEDTITYECPLLRQNEPEDIDCWCNSTSTWVTYGTCTTMGEHRREKRSVALVPHVGMGLETRTETWMSSEGAWKHVQRIETWILRHPGFTMMAAILAYTIGTTHFQRALIFILLTAVTPSMTMRCIGMSNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFRCKKNMEGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVTLVLVGIVTLYLGVMVQADSGCVVSWKNKELKCGSGIFITDNVHTWTEQYKFQPESPSKLASAIQKAHEEDICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTIPETILELTDALALGMMVLKMVRNMEKYQLAVTIMAILCVPNAVILQNAWKVSCTILAVVSVSPLFLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAVHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKARDALDNLAVLHTAEAGGRAYNHALSELPETLETLLLLTLLATVTGGIFLFLMSARGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAVFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 5 - DNA, 10717 bp, chimeric dengue serotype 2/3 (MVS)
agttgttagtctacgtggaccgacaaagacagattctttgagggagctaagctcaatgtagttctaacagttttttaattagagagcagatctctgatgaataaccaacggaaaaaggcgaaaaacacgcctttcaatatgctgaaacgcgagagaaaccgcgtgtcgactgtgcaacagctgacaaagagattctcacttggaatgctgcagggacgaggaccattaaaactgttcatggccctggtggcgttccttcgtttcctaacaatcccaccaacagcagggatattgaagagatggggaacaattaaaaaatcaaaagctattaatgttttgagagggttcaggaaagagattggaaggatgctgaacatcttgaataggagacgcagatctgcaggcatgatcattatgctgattccaacagtgatggcgttccatttaaccacgcgtgatggagagccgcgcatgattgtggggaagaatgaaagaggaaaatccctacttttcaagacagcctctggaatcaacatgtgcacactcatagctatggatctgggagagatgtgtgatgacacggtcacttacaaatgcccccacattaccgaagtggagcctgaagacattgactgctggtgcaaccttacatcgacatgggtgacttatggaacatgcaatcaagctggagagcatagacgcgataagagatcagtggcgttagctccccatgttggcatgggactggacacacgcactcaaacctggatgtcggctgaaggagcttggagacaagtcgagaaggtagagacatgggcccttaggcacccagggtttaccatactagccctatttcttgcccattacataggcacttccttgacccagaaagtggttatttttatactattaatgctggttaccccatccatgacaatgagatgtgtaggagtaggaaacagagattttgtggaaggcctatcgggagctacgtgggttgacgtggtgctcgagcacggtgggtgtgtgactaccatggctaagaacaagcccacgctggacatagagcttcagaagaccgaggccacccaactggcgaccctaaggaagctatgcattgagggaaaaattaccaacataacaaccgactcaagatgtcccacccaaggggaagcgattttacctgaggagcaggaccagaactacgtgtgtaagcatacatacgtggacagaggctggggaaacggttgtggtttgtttggcaagggaagcttggtgacatgcgcgaaatttcaatgtttagaatcaatagagggaaaagtggtgcaacatgagaacctcaaatacaccgtcatcatcacagtgcacacaggagaccaacaccaggtgggaaatgaaacgcagggagtcacggctgagataacaccccaggcatcaaccgctgaagccattttacctgaatatggaaccctcgggctagaatgctcaccacggacaggtttggatttcaatgaaatgatctcattgacaatgaagaacaaagcatggatggtacatagacaatggttctttgacttacccctaccatggacatcaggagcttcagcagaaacaccaacttggaacaggaaagagcttcttgtgacatttaaaaatgcacatgcaaaaaagcaagaagtagttgttcttggatcacaagagggagcaatgcatacagcactgacaggagctacagagatccaaacctcaggaggcacaagtatctttgcggggcacttaaaatgtagactcaagatggacaaattggaactcaaggggatgagctatgcaatgtgcttgagtagctttgtgttgaagaaagaagtctccgaaacgcagcatgggacaatactcattaaggttgagtacaaaggggaagatgcaccctgcaagattcctttctccacggaggatggacaaggaaaagctctcaatggcagactgatcacagccaatccagtggtgaccaagaaggaggagcctgtcaacattgaggctgaacctccttttggagaaagtaacatagtaattggaattggagacaaagccctgaaaatcaactggtacaagaagggaagctcgattgggaagatgttcgaggccactgccagaggtgcaaggcgcatggccatcttgggagacacagcctgggactttggatcagtgggtggtgttttgaattcattagggaaaatggtccaccaaatatttgggagtgcttacacagccctatttggtggagtctcctggatgatgaaaattggaataggtgtcctcttaacctggatagggttgaactcaaaaaatacttctatgtcattttcatgcatcgcggccggcattgtgacactgtatttgggagtcatggtgcaggccgatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagcccatgaagaggacatttgtggaatccgctcagtaacaagactggagaatctgatgtggaaacaaataacaccagaattgaatcacattctatcagaaaatgaggtgaagttaactattatgacaggagacatcaaaggaatcatgcaggcaggaaaacgatctctgcggcctcagcccactgagctgaagtattcatggaaaacatggggcaaagcaaaaatgctctctacagagtctcataaccagacctttctcattgatggccccgaaacagcagaatgccccaacacaaatagagcttggaattcgttggaagttgaagactatggctttggagtattcaccaccaatatatggctaaaattgaaagaaaaacaggatgtattctgcgactcaaaactcatgtcagcggccataaaagacaacagagccgtccatgccgatatgggttattggatagaaagtgcactcaatgacacatggaagatagagaaagcctctttcattgaagttaaaaactgccactggccaaaatcacacaccctctggagcaatggagtgctagaaagtgagatgataattccaaagaatctcgctggaccagtgtctcaacacaactatagaccaggctaccatacacaaataacaggaccatggcatctaggtaagcttgagatggactttgatttctgtgatggaacaacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaggagaaagaagagaatttggtcaactccttggtcacagctggacatgggcaggtcgacaacttttcactaggagtcttgggaatggcattgttcctggaggaaatgcttaggacccgagtaggaacgaaacatgcaatactactagttgcagtttcttttgtgacattgatcacagggaacatgtcctttagagacctgggaagagtgatggttatggtaggcgccactatgacggatgacataggtatgggcgtgacttatcttgccctactagcagccttcaaagtcagaccaacttttgcagctggactactcttgagaaagctgacctccaaggaattgatgatgactactataggaattgtactcctctcccagagcaccataccagagaccattcttgagttgactgatgcgttagccttaggcatgatggtcctcaaaatggtgagaaatatggaaaagtatcaattggcagtgactatcatggctatcttgtgcgtcccaaacgcagtgatattacaaaacgcatggaaagtgagttgcacaatattggcagtggtgtccgtttccccactgttcttaacatcctcacagcaaaaaacagattggataccattagcattgacgatcaaaggtctcaatccaacagctatttttctaacaaccctctcaagaaccagcaagaaaaggagctggccattaaatgaggctatcatggcagtcgggatggtgagcattttagccagttctctcctaaaaaatgatattcccatgacaggaccattagtggctggagggctcctcactgtgtgctacgtgctcactggacgatcggccgatttggaactggagagagcagccgatgtcaaatgggaagaccaggcagagatatcaggaagcagtccaatcctgtcaataacaatatcagaagatggtagcatgtcgataaaaaatgaagaggaagaacaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaattaagcaaaaagggattcttggatattcccagatcggagccggagtttacaaagaaggaacattccatacaatgtggcatgtcacacgtggcgctgttctaatgcataaaggaaagaggattgaaccatcatgggcggacgtcaagaaagacctaatatcatatggaggaggctggaagttagaaggagaatggaaggaaggagaagaagtccaggtattggcactggagcctggaaaaaatccaagagccgtccaaacgaaacctggtcttttcaaaaccaacgccggaacaataggtgctgtatctctggacttttctcctggaacgtcaggatctccaattatcgacaaaaaaggaaaagttgtgggtctttatggtaatggtgttgttacaaggagtggagcatatgtgagtgctatagcccagactgaaaaaagcattgaagacaacccagagatcgaagatgacattttccgaaagagaagactgaccatcatggacctccacccaggagcgggaaagacgaagagataccttccggccatagtcagagaagctataaaacggggtttgagaacattaatcttggcccccactagagttgtggcagctgaaatggaggaagcccttagaggacttccaataagataccagaccccagccatcagagctgtgcacaccgggcgggagattgtggacctaatgtgtcatgccacatttaccatgaggctgctatcaccagttagagtgccaaactacaacctgattatcatggacgaagcccatttcacagacccagcaagtatagcagctagaggatacatctcaactcgagtggagatgggtgaggcagctgggatttttatgacagccactcccccgggaagcagagacccatttcctcagagcaatgcaccaatcatagatgaagaaagagaaatccctgaacgctcgtggaattccggacatgaatgggtcacggattttaaagggaagactgtttggttcgttccaagtataaaagcaggaaatgatatagcagcttgcctgaggaaaaatggaaagaaagtgatacaactcagtaggaagacctttgattctgagtatgtcaagactagaaccaatgattgggacttcgtggttacaactgacatttcagaaatgggtgccaatttcaaggctgagagggttatagaccccagacgctgcatgaaaccagtcatactaacagatggtgaagagcgggtgattctggcaggacctatgccagtgacccactctagtgcagcacaaagaagagggagaataggaagaaatccaaaaaatgagaatgaccagtacatatacatgggggaacctctggaaaatgatgaagactgtgcacactggaaagaagctaaaatgctcctagataacatcaacacgccagaaggaatcattcctagcatgttcgaaccagagcgtgaaaaggtggatgccattgatggcgaataccgcttgagaggagaagcaaggaaaacctttgtagacttaatgagaagaggagacctaccagtctggttggcctacagagtggcagctgaaggcatcaactacgcagacagaaggtggtgttttgatggagtcaagaacaaccaaatcctagaagaaaacgtggaagttgaaatctggacaaaagaaggggaaaggaagaaattgaaacccagatggttggatgctaggatctattctgacccactggcgctaaaagaatttaaggaatttgcagccggaagaaagtctctgaccctgaacctaatcacagaaatgggtaggctcccaaccttcatgactcagaaggcaagagacgcactggacaacttagcagtgctgcacacggctgaggcaggtggaagggcgtacaaccatgctctcagtgaactgccggagaccctggagacattgcttttactgacacttctggctacagtcacgggagggatctttttattcttgatgagcgcaaggggcatagggaagatgaccctgggaatgtgctgcataatcacggctagcatcctcctatggtacgcacaaatacagccacactggatagcagcttcaataatactggagttttttctcatagttttgcttattccagaacctgaaaaacagagaacaccccaagacaaccaactgacctacgttgtcatagccatcctcacagtggtggccgcaaccatggcaaacgagatgggtttcctagaaaaaacgaagaaagatctcggattgggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtgtccctaacagctatagccaaccaagccacagtgttaatgggtctcgggaaaggatggccattgtcaaagatggacatcggagttccccttctcgccattggatgctactcacaagtcaaccccataactctcacagcagctcttttcttattggtagcacattatgccatcatagggccaggactccaagcaaaagcaaccagagaagctcagaaaagagcagcggcgggcatcatgaaaaacccaactgtcgatggaataacagtgattgacctagatccaataccttatgatccaaagtttgaaaagcagttgggacaagtaatgctcctagtcctctgcgtgactcaagtattgatgatgaggactacatgggctctgtgtgaggctttaaccttagctaccgggcccatctccacattgtgggaaggaaatccagggaggttttggaacactaccattgcggtgtcaatggctaacatttttagagggagttacttggccggagctggacttctcttttctattatgaagaacacaaccaacacaagaaggggaactggcaacataggagagacgcttggagagaaatggaaaagccgattgaacgcgttgggaaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgttgagagaaacatggtcacaccagaagggaaagtagtggacctcggttgtggcagaggaggctggtcatactattgtggaggactaaagaatgtaagagaagtcaaaggcctaacaaaaggaggaccaggacacgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtagaaaattggttgaacaacaacactcaattttgcataaaggttctcaacccatatatgccctcagtcatagaaaaaatggaagcactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtggtcaggctgctgacaaaaccttgggacgtcgtccccatggtgacacagatggcaatgacagacacgactccatttggacaacagcgcgtttttaaagagaaagtggacacgagaacccaagaaccgaaagaaggcacgaagaaactaatgaaaataacagcagagtggctttggaaagaattagggaagaaaaagacacccaggatgtgcaccagagaagaattcacaagaaaggtgagaagcaatgcagccttgggggccatattcactgatgagaacaagtggaagtcggcacgtgaggctgttgaagatagtaggttttgggagctggttgacaaggaaaggaatctccatcttgaaggaaagtgtgaaacatgtgtgtacaacatgatgggaaaaagagagaagaagctaggggaattcggcaaggcaaaaggcagcagagccatatggtacatgtggcttggagcacgcttcttagagtttgaagccctaggattcttaaatgaagatcactggttctccagagagaactccctgagtggagtggaaggagaagggctgcacaagctaggttacattctaagagacgtgagcaagaaagagggaggagcaatgtatgccgatgacaccgcaggatgggatacaagaatcacactagaagacctaaaaaatgaagaaatggtaacaaaccacatggaaggagaacacaagaaactagccgaggccattttcaaactaacgtaccaaaacaaggtggtgcgtgtgcaaagaccaacaccaagaggcacagtaatggacatcatatcgagaagagaccaaagaggtagtggacaagttggcacctatggactcaatactttcaccaatatggaagcccaactaatcagacagatggagggagaaggagtctttaaaagcattcagcacctaacaatcacagaagaaatcgctgtgcaaaactggttagcaagagtggggcgcgaaaggttatcaagaatggccatcagtggagatgattgtgttgtgaaacctttagatgacaggttcgcaagcgctttaacagctctaaatgacatgggaaagattaggaaagacatacaacaatgggaaccttcaagaggatggaatgattggacacaagtgcccttctgttcacaccatttccatgagttaatcatgaaagacggtcgcgtactcgttgttccatgtagaaaccaagatgaactgattggcagagcccgaatctcccaaggagcagggtggtctttgcgggagacggcctgtttggggaagtcttacgcccaaatgtggagcttgatgtacttccacagacgcgacctcaggctggcggcaaatgctatttgctcggcagtaccatcacattgggttccaacaagtcgaacaacctggtccatacatgctaaacatgaatggatgacaacggaagacatgctgacagtctggaacagggtgtggattcaagaaaacccatggatggaagacaaaactccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaagaatacacagattacatgccatccatgaaaagattcagaagagaagaggaagaagcaggagttctgtggtagaaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 6 - protein, 3389 aa, chimeric dengue serotype 2/3 (MVS)
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRRSAGMIIMLIPTVMAFHLTTRDGEPRMIVGKNERGKSLLFKTASGINMCTLIAMDLGEMCDDTVTYKCPHITEVEPEDIDCWCNLTSTWVTYGTCNQAGEHRRDKRSVALAPHVGMGLDTRTQTWMSAEGAWRQVEKVETWALRHPGFTILALFLAHYIGTSLTQKVVIFILLMLVTPSMTMRCVGVGNRDFVEGLSGATWVDVVLEHGGCVTTMAKNKPTLDIELQKTEATQLATLRKLCIEGKITNITTDSRCPTQGEAILPEEQDQNYVCKHTYVDRGWGNGCGLFGKGSLVTCAKFQCLESIEGKVVQHENLKYTVIITVHTGDQHQVGNETQGVTAEITPQASTAEAILPEYGTLGLECSPRTGLDFNEMISLTMKNKAWMVHRQWFFDLPLPWTSGASAETPTWNRKELLVTFKNAHAKKQEVVVLGSQEGAMHTALTGATEIQTSGGTSIFAGHLKCRLKMDKLELKGMSYAMCLSSFVLKKEVSETQHGTILIKVEYKGEDAPCKIPFSTEDGQGKALNGRLITANPVVTKKEEPVNIEAEPPFGESNIVIGIGDKALKINWYKKGSSIGKMFEATARGARRMAILGDTAWDFGSVGGVLNSLGKMVHQIFGSAYTALFGGVSWMMKIGIGVLLTWIGLNSKNTSMSFSCIAAGIVTLYLGVMVQADSGCVVSWKNKELKCGSGIFITDNVHTWTEQYKFQPESPSKLASAIQKAHEEDICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTIPETILELTDALALGMMVLKMVRNMEKYQLAVTIMAILCVPNAVILQNAWKVSCTILAVVSVSPLFLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAVHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKARDALDNLAVLHTAEAGGRAYNHALSELPETLETLLLLTLLATVTGGIFLFLMSARGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 7 - DNA, 10723 bp, chimeric dengue seroytpe 2/4 (MVS)
  r (3773)..(3773): a or g
agttgttagtctacgtggaccgacaaagacagattctttgagggagctaagctcaatgtagttctaacagttttttaattagagagcagatctctgatgaataaccaacggaaaaaggcgaaaaacacgcctttcaatatgctgaaacgcgagagaaaccgcgtgtcgactgtgcaacagctgacaaagagattctcacttggaatgctgcagggacgaggacctttaaaactgttcatggccctggtggcgttccttcgtttcctaacaatcccaccaacagcagggatattgaagagatggggaacaattaaaaaatcaaaagctattaatgttttgagagggttcaggaaagagattggaaggatgctgaacatcttgaataggagacgcagctctgcaggcatgatcattatgctgattccaacagtgatggcgttccatttaaccacgcgtgatggcgaacccctcatgatagtggcaaaacatgaaagggggagacctctcttgtttaagacaacagaggggatcaacaaatgcactctcattgccatggacttgggtgaaatgtgtgaggacactgtcacgtataaatgccccttactggtcaataccgaacctgaagacattgattgctggtgcaatctcacgtctacctgggtcatgtatgggacatgcacccagagcggagaacggagacgagagaagcgctcagtagctttaacaccacattcaggaatgggattggaaacaagagctgagacatggatgtcatcggaaggggcttggaagcatgctcagagagtagagagctggatactcagaaacccaggattcgcgctcttggcaggatttatggcttatatgattgggcaaacaggaatccagcgaactgtcttctttgtcctaatgatgctggtcgccccatcctacggaatgcgatgcgtaggagtaggaaacagagactttgtggaaggagtctcaggtggagcatgggtcgatctggtgctagaacatggaggatgcgtcacaaccatggcccagggaaaaccaaccttggattttgaactgactaagacaacagccaaggaagtggctctgttaagaacctattgcattgaagcctcaatatcaaacataaccacggcaacaagatgtccaacgcaaggagagccttatctaaaagaggaacaagaccaacagtacatttgccggagagatgtggtagacagagggtggggcaatggctgtggcttgtttggaaaaggaggagttgtgacatgtgcgaagttttcatgttcggggaagataacaggcaatttggtccaaattgagaaccttgaatacacagtggttgtaacagtccacaatggagacacccatgcagtaggaaatgacacgtccaatcatggagttacagccacgataactcccaggtcaccatcggtggaagtcaaattgccggactatggagaactaacactcgattgtgaacccaggtctggaattgactttaatgagatgattctgatgaaaatgaaaaagaaaacatggcttgtgcataagcaatggtttttggatctacctctaccatggacagcaggagcagacacatcagaggttcactggaattacaaagagagaatggtgacatttaaggttcctcatgccaagagacaggatgtgacagtgctgggatctcaggaaggagccatgcattctgccctcgctggagccacagaagtggactccggtgatggaaatcacatgtttgcaggacatctcaagtgcaaagtccgtatggagaaattgagaatcaagggaatgtcatacacgatgtgttcaggaaagttctcaattgacaaagagatggcagaaacacagcatgggacaacagtggtgaaagtcaagtatgaaggtgctggagctccgtgtaaagtccccatagagataagagatgtgaacaaggaaaaagtggttgggcgtatcatctcatccacccctttggctgagaataccaacagtgtaaccaacatagagttagaacccccctttggggacagctacatagtgataggtgttggaaacagtgcattaacactccattggttcaggaaagggagttccattggcaagatgtttgagtccacatacagaggtgcaaaacgaatggccattctaggtgaaacagcttgggattttggttccgttggtggactgttcacatcattgggaaaggctgtgcaccaggtttttggaagtgtgtatacaaccctgtttggaggagtctcatggatgattagaatcctaattgggttcctagtgttgtggattggcacgaactcaaggaacacttcaatggctatgacgtgcatagctgccggcattgtgacactgtatttgggggtcatggtgcaggccgatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagcccatgaagaggacatttgtggaatccgctcagtaacaagactggagaatctgatgtggaaacaaataacaccagaattgaatcacattctatcagaaaatgaggtgaagttaactattatgacaggagacatcaaaggaatcatgcaggcaggaaaacgatctctgcggcctcagcccactgagctgaagtattcatggaaaacatggggcaaagcaaaaatgctctctacagagtctcataaccagacctttctcattgatggccccgaaacagcagaatgccccaacacaaatagagcttggaattcgttggaagttgaagactatggctttggagtattcaccaccaatatatggctaaaattgaaagaaaaacaggatgtattctgcgactcaaaactcatgtcagcggccataaaagacaacagagccgtccatgccgatatgggttattggatagaaagtgcactcaatgacacatggaagatagagaaagcctctttcattgaagttaaaaactgccactggccaaaatcacacaccctctggagcaatggagtgctagaaagtgagatgataattccaaagaatctcgctggaccagtgtctcaacacaactatagaccaggctaccatacacaaataacaggaccatggcatctaggtaagcttgagatggactttgatttctgtgatggaacaacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaggagaaagaagagaatttggtcaactccttggtcacagctggacatgggcaggtcgacaacttttcactaggagtcttgggaatggcattgttcctggaggaaatgcttaggacccgagtaggaacgaaacatgcaatactactagttgcagtttcttttgtgacattgatcacagggaacatgtcctttagagacctgggaagagtgatggttatggtaggcgccactatgacgggtgacataggtatgggcgtgacttatcttgccctactagcagccttcaaagtcagaccaacttttgcagctggactactcttgagaaagctgacctccarggaattgatgatgactactataggaattgtactcctctcccagagcaccataccagagaccattcttgagttgactgatgcgttagccttaggcatgatggtcctcaaaatggtgagaaatatggaaaagtatcaattggcagtgactatcatggctatcttgtgcgtcccaaacgcagtgatattacaaaacgcatggaaagtgagttgcacaatattggcagtggtgtccgtttccccactgttcttaacatcctcacagcaaaaaacagattggataccattagcattgacgatcaaaggtctcaatccaacagctatttttctaacaaccctctcaagaaccagcaagaaaaggagctggccattaaatgaggctatcatggcagtcgggatggtgagcattttagccagttctctcctaaaaaatgatattcccatgacaggaccattagtggctggagggctcctcactgtgtgctacgtgctcactggacgatcggccgatttggaactggagagagcagccgatgtcaaatgggaagaccaggcagagatatcaggaagcagtccaatcctgtcaataacaatatcagaagatggtagcatgtcgataaaaaatgaagaggaagaacaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaattaagcaaaaagggattcttggatattcccagatcggagccggagtttacaaagaaggaacattccatacaatgtggcatgtcacacgtggcgctgttctaatgcataaaggaaagaggattgaaccatcatgggcggacgtcaagaaagacctaatatcatatggaggaggctggaagttagaaggagaatggaaggaaggagaagaagtccaggtattggcactggagcctggaaaaaatccaagagccgtccaaacgaaacctggtcttttcaaaaccaacgccggaacaataggtgctgtatctctggacttttctcctggaacgtcaggatctccaattatcgacaaaaaaggaaaagttgtgggtctttatggtaatggtgttgttacaaggagtggagcatatgtgagtgctatagcccagactgaaaaaagcattgaagacaacccagagatcgaagatgacattttccgaaagagaagactgaccatcatggacctccacccaggagcgggaaagacgaagagataccttccggccatagtcagagaagctataaaacggggtttgagaacattaatcttggcccccactagagttgtggcagctgaaatggaggaagcccttagaggacttccaataagataccagaccccagccatcagagctgtgcacaccgggcgggagattgtggacctaatgtgtcatgccacatttaccatgaggctgctatcaccagttagagtgccaaactacaacctgattatcatggacgaagcccatttcacagatccagcaagtatagcagctagaggatacatctcaactcgagtggagatgggtgaggcagctgggatttttatgacagccactcccccgggaagcagagacccatttcctcagagcaatgcaccaatcatagatgaagaaagagaaatccctgaacgctcgtggaattccggacatgaatgggtcacggattttaaagggaagactgtttggttcgttccaagtataaaagcaggaaatgatatagcagcttgcctgaggaaaaatggaaagaaagtgatacaactcagtaggaagacctttgattctgagtatgtcaagactagaaccaatgattgggacttcgtggttacaactgacatttcagaaatgggtgccaatttcaaggctgagagggttatagaccccagacgctgcatgaaaccagtcatactaacagatggtgaagagcgggtgattctggcaggacctatgccagtgacccactctagtgcagcacaaagaagagggagaataggaagaaatccaaaaaatgagaatgaccagtacatatacatgggggaacctctggaaaatgatgaagactgtgcacactggaaagaagctaaaatgctcctagataacatcaacacgccagaaggaatcattcctagcatgttcgaaccagagcgtgaaaaggtggatgccattgatggcgaataccgcttgagaggagaagcaaggaaaacctttgtagacttaatgagaagaggagacctaccagtctggttggcctacagagtggcagctgaaggcatcaactacgcagacagaaggtggtgttttgatggagtcaagaacaaccaaatcctagaagaaaacgtggaagttgaaatctggacaaaagaaggggaaaggaagaaattgaaacccagatggttggatgctaggatctattctgacccactggcgctaaaagaatttaaggaatttgcagccggaagaaagtctctgaccctgaacctaatcacagaaatgggtaggctcccaaccttcatgactcagaaggtaagagacgcactggacaacttagcagtgctgcacacggctgaggcaggtggaagggcgtacaaccatgctctcagtgaactgccggagaccctggagacattgcttttactgacacttctggctacagtcacgggagggatctttttattcttgatgagcgcaaggggcatagggaagatgaccctgggaatgtgctgcataatcacggctagcatcctcctatggtacgcacaaatacagccacactggatagcagcttcaataatactggagttttttctcatagttttgcttattccagaacctgaaaaacagagaacaccccaagacaaccaactgacctacgttgtcatagccatcctcacagtggtggccgcaaccatggcaaacgagatgggtttcctagaaaaaacgaagaaagatctcggattgggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtgtccctaacagccatagccaaccaagccacagtgttaatgggtctcgggaaaggatggccattgtcaaagatggacatcggagttccccttctcgccattggatgctactcacaagtcaaccccataactctcacagcagctcttttcttattggtagcacattatgccatcatagggccaggactccaagcaaaagcaaccagagaagctcagaaaagagcagcggcgggcatcatgaaaaacccaactgtcgatggaataacagtgattgacctagatccaataccttatgatccaaagtttgaaaagcagttgggacaagtaatgctcctagtcctctgcgtgactcaagtattgatgatgaggactacatgggctctgtgtgaggctttaaccttagctaccgggcccatctccacattgtgggaaggaaatccagggaggttttggaacactaccattgcggtgtcaatggctaacatttttagagggagttacttggccggagctggacttctcttttctattatgaagaacacaaccaacacaagaaggggaactggcaacataggagagacgcttggagagaaatggaaaagccgattgaacgcattgggaaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgttgagagaaacatggtcacaccagaagggaaagtagtggacctcggttgtggcagaggaggctggtcatactattgtggaggactaaagaatgtaagagaagtcaaaggcctaacaaaaggaggaccaggacacgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtagaaaattggttgaacaacaacactcaattttgcataaaggttctcaacccatatatgccctcagtcatagaaaaaatggaagcactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtggtcaggctgctgacaaaaccttgggacgtcgtccccatggtgacacagatggcaatgacagacacgactccatttggacaacagcgcgtttttaaagagaaagtggacacgagaacccaagaaccgaaagaaggcacgaagaaactaatgaaaataacagcagagtggctttggaaagaattagggaagaaaaagacacccaggatgtgcaccagagaagaattcacaagaaaggtgagaagcaatgcagccttgggggccatattcactgatgagaacaagtggaagtcggcacgtgaggctgttgaagatagtaggttttgggagctggttgacaaggaaaggaatctccatcttgaaggaaagtgtgaaacatgtgtgtacaacatgatgggaaaaagagagaagaagctaggggaattcggcaaggcaaaaggcagcagagccatatggtacatgtggcttggagcacgcttcttagagtttgaagccctaggattcttaaatgaagatcactggttctccagagagaactccctgagtggagtggaaggagaagggctgcacaagctaggttacattctaagagacgtgagcaagaaagagggaggagcaatgtatgccgatgacaccgcaggatgggatacaagaatcacactagaagacctaaaaaatgaagaaatggtaacaaaccacatggaaggagaacacaagaaactagccgaggccattttcaaactaacgtaccaaaacaaggtggtgcgtgtgcaaagaccaacaccaagaggcacagtaatggacatcatatcgagaagagaccaaagaggtagtggacaagttggcacctatggactcaatactttcaccaatatggaagcccaactaatcagacagatggagggagaaggagtctttaaaagcattcagcacctaacaatcacagaagaaatcgctgtgcaaaactggttagcaagagtggggcgcgaaaggttatcaagaatggccatcagtggagatgattgtgttgtgaaacctttagatgacaggttcgcaagcgctttaacagctctaaatgacatgggaaagattaggaaagacatacaacaatgggaaccttcaagaggatggaatgattggacacaagtgcccttctgttcacaccatttccatgagttaatcatgaaagacggtcgcgtactcgttgttccctgtagaaaccaagatgaactgattggcagagcccgaatctcccaaggagcagggtggtctttgcgggagacggcctgtttggggaagtcttacgcccaaatgtggagcttgatgtacttccacagacgcgacctcaggctggcggcaaatgctatttgctcggcagtaccatcacattgggttccaacaagtcgaacaacctggtccatacatgctaaacatgaatggatgacaacggaagacatgctgacagtctggaacagggtgtggattcaagaaaacccatggatggaagacaaaactccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaagaatacacagattacatgccatccatgaaaagattcagaagagaagaggaagaagcaggagttctgtggtagaaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 8 - protein, 3391 aa, chimeric dengue serotype 2/4 (MVS)
  Xaa (1226)..(1226): Arg or Lys
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRSSAGMIIMLIPTVMAFHLTTRDGEPLMIVAKHERGRPLLFKTTEGINKCTLIAMDLGEMCEDTVTYKCPLLVNTEPEDIDCWCNLTSTWVMYGTCTQSGERRREKRSVALTPHSGMGLETRAETWMSSEGAWKHAQRVESWILRNPGFALLAGFMAYMIGQTGIQRTVFFVLMMLVAPSYGMRCVGVGNRDFVEGVSGGAWVDLVLEHGGCVTTMAQGKPTLDFELTKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVTATITPRSPSVEVKLPDYGELTLDCEPRSGIDFNEMILMKMKKKTWLVHKQWFLDLPLPWTAGADTSEVHWNYKERMVTFKVPHAKRQDVTVLGSQEGAMHSALAGATEVDSGDGNHMFAGHLKCKVRMEKLRIKGMSYTMCSGKFSIDKEMAETQHGTTVVKVKYEGAGAPCKVPIEIRDVNKEKVVGRIISSTPLAENTNSVTNIELEPPFGDSYIVIGVGNSALTLHWFRKGSSIGKMFESTYRGAKRMAILGETAWDFGSVGGLFTSLGKAVHQVFGSVYTTLFGGVSWMIRILIGFLVLWIGTNSRNTSMAMTCIAAGIVTLYLGVMVQADSGCVVSWKNKELKCGSGIFITDNVHTWTEQYKFQPESPSKLASAIQKAHEEDICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTGDIGMGVTYLALLAAFKVRPTFAAGLLLRKLTSXELMMTTIGIVLLSQSTIPETILELTDALALGMMVLKMVRNMEKYQLAVTIMAILCVPNAVILQNAWKVSCTILAVVSVSPLFLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAVHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKVRDALDNLAVLHTAEAGGRAYNHALSELPETLETLLLLTLLATVTGGIFLFLMSARGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW